SEQUENCE LISTING (26 sequences)

SEQ ID NO: 1 - protein, 4 aa, Artificial Sequence
  Synthetic Sequence
AGAS

SEQ ID NO: 2 - protein, 4 aa, Artificial Sequence
  Synthetic Sequence
DSGH

SEQ ID NO: 3 - protein, 4 aa, Artificial Sequence
  Synthetic Sequence
RAHE

SEQ ID NO: 4 - protein, 4 aa, Artificial Sequence
  Synthetic Sequence
KEDV

SEQ ID NO: 5 - protein, 8 aa, Artificial Sequence
  Synthetic Sequence
DYKDDDDK

SEQ ID NO: 6 - protein, 4 aa, Artificial Sequence
  Synthetic Sequence
DAHK

SEQ ID NO: 7 - protein, 4 aa, Artificial Sequence
  Synthetic Sequence
HAMR

SEQ ID NO: 8 - protein, 4 aa, Artificial Sequence
  Synthetic Sequence
HAHN

SEQ ID NO: 9 - protein, 4 aa, Artificial Sequence
  Synthetic Sequence
DTHK

SEQ ID NO: 10 - protein, 4 aa, Artificial Sequence
  Synthetic Sequence
AYFE

SEQ ID NO: 11 - protein, 4 aa, Artificial Sequence
  Synthetic Sequence
GFWY

SEQ ID NO: 12 - protein, 4 aa, Artificial Sequence
  Synthetic Sequence
QWFA

SEQ ID NO: 13 - protein, 4 aa, Artificial Sequence
  Synthetic Sequence
HWWM

SEQ ID NO: 14 - protein, 4 aa, Artificial Sequence
  Synthetic Sequence
DAPS

SEQ ID NO: 15 - protein, 4 aa, Artificial Sequence
  Synthetic Sequence
ESRE

SEQ ID NO: 16 - protein, 4 aa, Artificial Sequence
  Synthetic Sequence
ESTQ

SEQ ID NO: 17 - protein, 4 aa, Artificial Sequence
  Synthetic Sequence
AAHA

SEQ ID NO: 18 - protein, 4 aa, Artificial Sequence
  Synthetic Sequence
CHPG

SEQ ID NO: 19 - DNA, 19 bp, Artificial Sequence
  Synthetic Sequence
cttgtgcagtgccagcctc

SEQ ID NO: 20 - DNA, 20 bp, Artificial Sequence
  Synthetic Sequence
accagcttcccattctcagc

SEQ ID NO: 21 - DNA, 20 bp, Artificial Sequence
  Synthetic Sequence
gacttcacgggcaccattca

SEQ ID NO: 22 - protein, 20 aa, Artificial Sequence
  Synthetic Sequence
GAACACCCTTACCAAGCGGA

SEQ ID NO: 23 - DNA, 21 bp, Artificial Sequence
  Synthetic Sequence
gagcaagcgcgttgttattga

SEQ ID NO: 24 - DNA, 21 bp, Artificial Sequence
  Synthetic Sequence
gtgctttctcctcctcatgct

SEQ ID NO: 25 - DNA, 20 bp, Artificial Sequence
  Synthetic Sequence
ggttttatgcaccagctgcc

SEQ ID NO: 26 - DNA, 22 bp, Artificial Sequence
  Synthetic Sequence
atgttgatcatgccatctccag

(19) United States
(12) Patent Application Publication
(10) Pub. No.: US 2023/0140798 A1
(43) Pub. Date: May 4, 2023

(54) SYNTHETIC PEPTIDES DIRECTED AGAINST THE METABOTROPIC GLUTAMATE RECEPTOR 5

(71) Applicant: Lactocore, Inc., Plymouth, MI (US)

(72) Inventors: Anton MALYSHEV, Plymouth, MI (US); Igor DORONIN, Plymouth, MI (US); Gennady BABKIN, Plymouth, MI (US); Askar KUCHUMOV, Plymouth, MI (US)

(21) Appl. No.: 17/996,144

(22) PCT Filed: Apr. 15, 2021
(86) PCT No.: PCT/US21/27486

§ 371 Date: Oct. 13, 2022

Related U.S. Application Data
(60) Provisional application No. 63/010,425, filed on Apr. 15, 2020.

(51) Int. Cl. C07K 5/10; A61P 25/14; A61P 25/28

(52) U.S. Cl. CPC C07K 5/10; A61P 25/14; A61P 25/28

(57) ABSTRACT

A pharmaceutical composition comprising a synthetic neuromodulatory peptide is described. The invention discloses neuromodulatory peptides as defined in the claims and methods of using such molecules for therapeutic application. The neuromodulatory peptides included in the composition have been found to be effective in treatment of mood disorders and movement disorders, including movement disorders accompanying mood and mental disorders.

### PRIORITY

The present application claims priority to and benefit from the U.S. Provisional Patent Application No. 63/010,425, filed Apr. 15, 2020, the entire content of which is incorporated by reference herein.

### FIELD

The present disclosure relates to compositions that include proteins, such as peptide therapeutic agents, to treat depression and other psychiatric disorders as well as movement disorders.

### DESCRIPTION OF THE TEXT FILE SUBMITTED ELECTRONICALLY

This application contains a Sequence Listing in ASCII format submitted electronically herewith via EFS-Web. The ASCII copy, created on Apr. 14, 2021, is named LACT-002PC_ST25.txt and is 4,757 bytes in size. The Sequence Listing is incorporated herein by reference in its entirety.

### BACKGROUND

Depression, which manifests in many conditions affecting a subject's mood, affects millions of people worldwide and its treatment is still generally inadequate. Although various drugs for treatment of depression and associated conditions have been developed, the drugs are typically not specific enough and are ineffective for about 40 percent of patients. Also, it usually takes weeks before a patient can benefit from a therapeutic action of a drug. Moreover, many known drugs have various side effects. Dystonia, akathisia, parkinsonism, and choreiform dyskinesia are common side effects of antipsychotic medications. Although originally associated with classical antipsychotic agents such as chlorpromazine or haloperidol, they have also been described in association with a wide range of other psychotropic drugs, including all the atypical neuroleptics, antidepressants (both tricyclic and SSRI) and anticonvulsants such as sodium valproate. See Lennox et al. (2002). Mind and movement: the neuropsychiatry of movement disorders. J. Neurol. Neurosurg. Psychiatry. 72(s1): i28-i31.

An anxiety disorder, although different from depression, often accompanies depression. Many anxiolytic drugs have issues similar to antidepressants. The challenge in discovering effective treatments for depression and anxiety includes identifying appropriate targets to upregulate or downregulate. Another challenge is to design safe, low cost therapeutics that are specific to those targets and that are able to alleviate depression and anxiety symptoms within a relatively short timeframe. Another comorbid symptom of depression is psychomotor retardation—a core feature of major depressive disorder, which indicates a more severe disorder with a poorer prognosis. And at the same time, major movement disorders (such as Parkinson's disease, idiopathic dystonia, Huntington's disease, and Gilles de la Tourette's syndrome, essential tremor) have important psychiatric dimensions, which suggests a need for the complex treatment interventions of both psychiatric and neurological manifestations of these diseases. Accordingly, there remains a need to develop effective and safe therapeutics for treatment of depression, movement disorders and associated diseases.

### SUMMARY

In various aspects, the present invention provides compositions and methods that are useful for treatment of various mental, behavioral, affective, neurotic, movement and emotional disorders, including depression, anxiety, stress-related disorders as well as hypo-, hyper- and bradykinesias. In some aspects, a synthetic neuromodulatory peptide, such as, for example, tetrapeptide, in the form of a pharmaceutical composition can be used for treatment of depression and other mood or locomotor disorders.

In some embodiments, a composition is provided that comprises a synthetic neuromodulatory peptide, that is defined by the general formula I:

R₁R₂R₃R₄ (I), wherein at least one of R₁-R₄ is hydrophobic and at least one of R₁-R₄ is polar or charged; none of R₁-R₄ is selected from L, M, I, T, C, P, N, Q, F, Y, and W; and the peptide modulates the mGluR₅ receptor (GRM₅).

In some embodiments, R₁ is D, R₂ is S, R₃ is G, and R₄ is H. In some embodiments, R₁ is R, R₂ is A, R₃ is H, and R₄ is E. In some embodiments, R₁ is K, R₂ is E, R₃ is D, and R₄ is V. In some embodiments, R₁ is A, R₂ is G, R₃ is A, and R₄ is S.

The neuromodulatory peptides and their analogs described herein are developed to modulate mGluR₅ receptors (GRM5). In view of the known link between GRM5 receptors and psychiatric disorders, including anxiety and depression, the neuromodulatory peptide of the present disclosure is effective at preventing or treating various depression-anxiety spectrum disorders as well as movement disorders, including Parkinson's disease. Non-limiting examples of conditions that can be treated using the described neuromodulatory peptide include generalized anxiety disorder (GAD), post-traumatic stress disorder (PTSD), major depressive disorder (MDD), treatment-resistant depression (TRD), postpartum depression (PPD), bipolar disorder or bipolar depression, obsessive-compulsive disorder (OCD), and schizophrenia. Movement disorders (MD) that can be treated using the described neuromodulatory peptide include hypokinetic MD, such as Parkinson's disease (primary or idiopathic and secondary) and Parkinson plus syndromes, hyperkinetic MD, such as dystonia (drug induced dystonia and idiopathic dystonia in particular), dyskinesia (e.g., tardive or levodopa-induced dyskinesia), essential tremor, Huntington's chorea, Tourette's syndrome, stereotypic movement disorder and such mental disorders with movement component as attention deficit hyperactivity disorder (ADHD). In some embodiments, the movement disorder is a hypokinetic movement disorder or hyperkinetic movement disorder. In some embodiments, the movement disorder accompanies a mental disorder.

In some aspects, the tetrapeptides can be optionally chemically modified. The chemical modification can be selected from amidation, methylation, and acetylation of one or more of the amino acids. Additional chemical modifications can include addition of formyl, pyroglutamyl (pGlu), one or more fatty acids, urea, carbamate, sulfonamide, alkylamine, or any combination thereof. The composition can include a pharmaceutically acceptable carrier. In some embodiments, the composition can further include a delivery vehicle which can be, e.g., a liposome, a nanoparticle, or a polysaccharide. The composition can be administered to a subject determined to be in need of treatment via various routes, and in some aspects the composition is formulated for intranasal administration.

### BRIEF DESCRIPTION OF THE FIGURES

FIG. 1 illustrates the apparatuses for Novel Tank (NT) test.

FIG. 2 illustrates the apparatuses for Light-Dark Box (LDB) test.

FIGS. 3A-3H illustrate the behavioral effects of ketamine (“Ket”) in NT (A-E) and LDB (F-H) tests. FIG. 3A—time spent at the top of the tank (“Time surface,” %) FIG. 3B—latency to enter the top of the tank (“LP to the top,” %); FIG. 3C—time spent in the upper ⅔ of aquarium (top+middle zones) (“Time TOP+Middle,” %); FIG. 3D—distance travelled (“Distance,” %); FIG. 3E—velocity (“Velocity,” %); FIG. 3F—time spent in the light compartment (“Time on light,” %); FIG. 3G—latency to enter the light compartment (“LP to the light,” %); FIG. 3H—the number of transitions (“Transitions to light,” %). The data shown as % from control values. The results are expressed as the mean±SEM. *p<0.05 represents significant differences vs. control group (Mann-Whitney U-test).

FIGS. 4A-4H illustrate the behavioral effects of AGAS (SEQ ID NO: 1) treatment at different doses in NT (A-E) and LDB (F-H) tests. FIG. 4A—time spent at the top of the tank; FIG. 4B—latency to enter the top of the tank; FIG. 4C—time spent in the upper ⅔ of aquarium (top+middle zones); FIG. 4D—distance travelled; FIG. 4E—velocity; FIG. 4F—time spent in the light compartment; FIG. 4G—latency to enter the light compartment; FIG. 4H—the number of transitions. The data shown as % from control values. The results are expressed as the mean±SEM. *p<0.05 represents significant differences vs. corresponding control group (Mann-Whitney U-test).

FIGS. 5A-5H illustrate the behavioral effects of DSGH (SEQ ID NO: 2) treatment at different doses in NT (A-E) and LDB (F-H) tests. FIG. 5A—time spent at the top of the tank; FIG. 5B—latency to enter the top of the tank; FIG. 5C—time spent in the upper ⅔ of aquarium (top+middle zones); FIG. 5D—distance travelled; FIG. 5E—velocity; FIG. 5F—time spent in the light compartment; FIG. 5G—latency to enter the light compartment; FIG. 5H—the number of transitions. The data shown as % from control values. The results are expressed as the mean±SEM. *p<0.05 represents significant differences vs. corresponding control group (Mann-Whitney U-test).

FIGS. 6A-6E illustrate the behavioral effects of RAHE (SEQ ID NO: 3) treatment at different doses in NT (A-E) test. FIG. 6A—time spent at the top of the tank; FIG. 6B—latency to enter the top of the tank; FIG. 6C—time spent in the upper ⅔ of aquarium (top+middle zones); FIG. 6D—distance travelled; FIG. 6E—velocity. The data shown as % from control values. The results are expressed as the mean±SEM. *p<0.05 represents significant differences vs. corresponding control group (Mann-Whitney U-test).

FIGS. 7A-7H illustrate the behavioral effects of KEDV (SEQ ID NO: 4) treatment at different doses in NT (A-E) and LDB (F-H) tests. FIG. 7A—time spent at the top of the tank; FIG. 7B—latency to enter the top of the tank; FIG. 7C—time spent in the upper ⅔ of aquarium (top+middle zones); FIG. 7D—distance travelled; FIG. 7E—velocity; FIG. 7F—time spent in the light compartment; FIG. 7G—latency to enter the light compartment; FIG. 7H—the number of transitions. The data shown as % from control values. The results are expressed as the mean±SEM. *p<0.05 represents significant differences vs. corresponding control group (Mann-Whitney U-test).

FIGS. 8A-8E illustrate the behavioral effects of AYFE (SEQ ID NO: 10) treatment at different doses in a NT test. FIG. 8A—time spent at the top of the tank; FIG. 8B—latency to enter the top of the tank; FIG. 8C—time spent in the upper ⅔ of aquarium (top+middle zones); FIG. 8D—distance travelled; FIG. 8E—velocity. The data shown as % from control values. The results are expressed as the mean±SEM.

FIG. 9 illustrates the 2D trajectories of Danio rerio from the control group (left panel) and a ketamine-treated group (right panel). Darker lines represent the part of the track located at the “top” of the aquarium.

FIG. 10 illustrates the summary of behavioral effects after treatment with ketamine and studied tetrapeptides at different doses. “G” arrows represent anxiolytic-like effect, “r”—anxiogenic-like effects, “y”—hyperactivity or enhanced exploratory activity, “b”—sedative effect produced by the drug.

FIGS. 11A, 11B, 11C and 11D illustrate the behavior of BALB/C mice in the Open Field test 30 minutes after intraperitoneal drug injection. FIG. 11A. Total distance travelled, cm. FIG. 11B. Number of rears. FIG. 11C. Number of center entries. FIG. 11D. Time spent in the center. The results are expressed as the mean±SEM. *p<0.05 represents significant differences vs. control group. One-way ANOVA with Fisher's LSD post hoc test.

FIGS. 12A, 12B, 12C, and 12D illustrate the behavior of BALB/C mice in the Elevated Plus Maze 30 minutes after intraperitoneal drug injection. FIG. 12A. Distance travelled, cm. FIG. 12B. Freezing time, s. FIG. 12C. Number of rears. FIG. 12D. Number of open arms entries. The results are expressed as the mean±SEM. *p<0.05 represents significant differences vs. control group. One-way ANOVA with Fisher's LSD post hoc test.

FIGS. 13A, 13B, and 13C illustrate the behavior of BALB/C mice in the Porsolt Forced Swim test (two-day modification) 30 minutes after intraperitoneal drug injection. FIG. 13A. Time of active swimming, s. FIG. 13B. Time of passive swimming, s. FIG. 13C. Immobility time, s. The results are expressed as the mean±SEM. *p<0.05 represents significant differences vs. control group and #p<0.05 vs. Fluvoxamine group (FA). One-way ANOVA with Fisher's LSD post hoc test.

FIGS. 14A, 14B and 14C illustrate the behavior of Sprague-Dawley rats in the Open Field test 30 minutes after intranasal drug administration. FIG. 14A. Total distance travelled, cm. FIG. 14B. Number of center entries. FIG. 14C. Time spent in the center, s. The results are expressed as the mean±SEM. *p<0.05 represents significant differences vs. control group. One-way ANOVA with Fisher's LSD post hoc test.

FIGS. 15A, 15B and 15C illustrate the behavior of Sprague-Dawley rats in the Novelty Suppressed Feeding test 30 minutes after intranasal drug administration. FIG. 15A. Latency to eat, s. FIG. 15B. Time spent eating, s. FIG. 15C. Distance travelled, cm. The results are expressed as the mean±SEM. *p<0.05 represents significant differences vs. control group. One-way ANOVA with Fisher's LSD post hoc test.

FIG. 16 illustrates the novel object preference (%) of Sprague-Dawley rats in the Novel Object Recognition test 30 minutes after intranasal drug administration on the training day. Significant effect for factor “day of the experiment” F_{1, 63}=16.01; p=0.0001, according to repeated measures ANOVA. The results are expressed as the mean±SEM.

FIGS. 17A, 17B, 17C, and 17D illustrate the behavior of Sprague-Dawley rats in the Elevated Plus Maze 30 minutes after intranasal drug administration. FIG. 17A. Time spent in the open arms, s. FIG. 17B. Open arm entries. FIG. 17C. Anxiety Index (AI), %. FIG. 17D. Distance travelled, cm. The results are expressed as the mean±SEM. *p<0.05 represents significant differences vs. control group. One-way ANOVA with Fisher's LSD post hoc test.

FIGS. 18A, 18B, and 18C illustrate the behavior of Sprague-Dawley rats in the Porsolt Forced Swim test (two-day modification) 30 minutes after intranasal drug administration. FIG. 18A. Time of active swimming, s. FIG. 18B. Time of passive swimming, s. FIG. 18C. Immobility time, s. The results are expressed as the mean±SEM.

FIG. 19 illustrates principle of mGluR₅ (GRM5) luciferase assay.

FIG. 20 illustrates the results of HEK 293 cells mGluR₅-luciferase reporter assay with CHPG treatments. The cells were co-administered with selective noncompetitive antagonist of mGluR₅ SIB 1757 at a dose of 10 and 100 μM, RAHE (SEQ ID NO: 3) and KEDV (SEQ ID NO: 4) peptides at a dose of 0.2, 2 and 20 μM. The data are presented as the means±SE for 3 biological replicates. *p<0.05 represents significant difference in comparison with “Transfected cells+CHPG” group according to Student's T-test.

FIG. 21 illustrates the results of HEK 293 cells mGluR₅-luciferase reporter assay with CHPG treatments or RAHE (SEQ ID NO: 3) and KEDV (SEQ ID NO: 4) alone at a dose of 20 and 200 μM. The CHPG-treated cells were co-administered with selective noncompetitive antagonist of mGluR₅ SIB 1757 at a dose of 10 μM or RAHE (SEQ ID NO: 3) and KEDV (SEQ ID NO: 4) peptides at a dose of 20, 100 and 200 μM or RAHE (SEQ ID NO: 3) and KEDV (SEQ ID NO: 4) (200 μM) together with SIB 1757 (10 μM). The data are presented as the means±SE for 3 biological replicates. *p<0.05 represents significant difference in comparison with “Transfected cells+CHPG” group according to Student's T-test.

FIG. 22 illustrates the motor activity of Wistar rats in the Locomotor Activity Test during 30-minute intervals (arbitrary units). The results are expressed as the mean±SE. *−p<0.05 relative to the control group (saline administration), repeated-measures ANOVA with post hoc Fisher's LSD test.

FIGS. 23A and 23B illustrate the severity of sensorimotor deficits in the Beam Walking test (BWT) in Wistar rats. FIG. 23A. Severity of sensorimotor deficits (front limbs), %. FIG. 23B. Severity of sensorimotor deficits (hind limbs), %. The results are expressed as the mean±SE. *−p<0.05 relative to the control group (saline administration). One-way ANOVA with Fisher's LSD post hoc test.

FIGS. 24A, 24B, and 24C illustrate mRNA relative expression levels in frontal cortex of Wistar rats (normalized to GAPDH household gene). FIG. 24A. Relative expression of Camk2n1 mRNA. FIG. 24B. Relative expression of Kcna1 mRNA. FIG. 24C. Relative expression of Egr2 mRNA. The results are expressed as the mean±SE. *p<0.05 represents significant differences vs. control group. One-way ANOVA with Fisher's LSD post hoc test.

FIGS. 25A, 25B, 25C, and 25D illustrate locomotion of Wistar rats in Spontaneous Motor Activity Test during 10-30-minute intervals (arbitrary units). FIG. 25A. Locomotion during first 10-min of the experiment. FIG. 25B. Locomotion during 10-40 min of the experiment. FIG. 25C. Locomotion during 40-70 min of the experiment. FIG. 25D. Locomotion during 70-100 min of the experiment. The results are expressed as the mean±SE. The vertical axis represents arbitrary units, reflecting the number of motor acts rats per minute (mean value on the interval). *−p<0.05 relative to the control group (repeated measures ANOVA with a post hoc Fisher's LSD test).

FIG. 26 illustrates freezing duration in the Open Field Test, sec. The results are expressed as the mean±SE. One-way ANOVA with post hoc Fisher's LSD test [F(6, 56)=3.6, p=0.004]. *−p<0.05 vs. control group, #−p<0.05 vs. “AFS+veh” group.

FIG. 27 illustrates the proportion (in %) of animals in the experimental groups that visited (entries) and did not visit (no entries) the open arms in the EPM test. *p<0.05—significant differences from the control group, #p<0.05—from the “AFS+veh” group. X2 test with Yates' correction.

FIG. 28 illustrates serum corticosterone concentrations after vehicle or dexamethasone (DXMT) injection, nmol/L. The results are expressed as the mean±SE. Repeated measures ANOVA [F (6, 56)=3.1, p=0.01; Fisher's LSD test]. *−p<0.05 vs corresponding “veh” in each treatment group.

FIGS. 29A, 29B, 29C, 29D, 29E, and 29F illustrate [Ca²+] responses of CHO-mGluR5 cells to 1 mM sodium glutamate (Glu-Na) in the presence of different concentrations of KEDV (SEQ ID NO: 4), RAHE (SEQ ID NO: 3) or MPEP. FIG. 29A. Example of [Ca²+] currents after application of KEDV (SEQ ID NO: 4) in a concentration of 0.02, 2, 20 and 200 μM. FIG. 29B. Example of [Ca²+] currents after application of RAHE (SEQ ID NO: 3) in a concentration of 0.02, 2, 20 and 200 μM. FIG. 29C. Example of [Ca²+] currents after application of MPEP in a concentration of 0.1, 1, 10 and 100 μM. [Ca²+] currents are measured as changes in fluorescence intensity before (FI_{base}) and after Glu-Na addition (FI). The data shown are representative average plots (n=3) of normalized fluorescence signals against time during assays. Each average plot is normalized to average FI_{base} at 0 sec time point for the respective plot. FIG. 29D. Inhibitory effects of KEDV (SEQ ID NO: 4) on intracellular [Ca²+] levels at the peak CHO-mGluR5 cells activation (27 seconds after Glu-Na application). FIG. 29E. Inhibitory effects of RAHE (SEQ ID NO: 3) on intracellular [Ca²+] levels at the peak CHO-mGluR5 cells activation (27 seconds after Glu-Na application). FIG. 29F. Inhibitory effects of MPEP on intracellular [Ca²+] levels at the peak CHO-mGluR5 cells activation (27 seconds after Glu-Na application). The value of 1 on the ordinate axis is the baseline level of fluorescence before activation (FI_{base} at 0 sec time point). Statistical analysis was performed using unpaired t-test. *−p<0.05 in respect to positive control with Glu-Na; #−p<0.05 in respect to negative control.

### DETAILED DESCRIPTION

The peptide compositions are provided herein, which have use in, for instance, treatment of depression, anxiety, associated mood conditions, stress-related and movement disorders. In some aspects, peptide-based neuromodulatory therapeutical compositions for a range of psychiatric and neurological conditions within the spectrum of depressive, anxiety and movement disorders were developed. Central nervous system (CNS) targets were selected to achieve high specificity and efficacy of neuromodulatory peptide compositions. In combination with anticipated safety profile of peptides, the compositions in accordance with the present disclosure provide safe and effective treatment.

In embodiments in accordance with the present disclosure, a GRM5 receptor, which is a metabotropic receptor, was selected as a target for the described group of neuromodulatory peptides. The endogenous ligand of GRMs receptors is glutamate which is the major excitatory neurotransmitter in the CNS. GRM5 is a functional homodimer and a member of G-protein coupled receptor (GPCR) Class 3. GRM5 possesses the typical GPCR seven transmembrane-spanning regions that are connected by three intracellular and three extracellular loops. It has a large, bilobed extracellular N-terminal domain, which contains binding sites for orthosteric agonists. The GRMs modulate neurotransmitter release and postsynaptic excitatory neurotransmission and hence modulate the strength of the transmission. GRM5 is widely expressed throughout the CNS. GRM5 antagonists show profound activities in anxiolytic and antidepressant tests (Carroll et al. (2008). Antagonists at metabotropic glutamate receptor subtype 5: structure activity relationships and therapeutic potential for addiction. Ann. N. Y Acad. Sci. 1141(1): 221-232). A major breakthrough in the area of GRM5 biology came with the discovery of highly selective allosteric antagonists of mGluR₅, including 2-methyl-6-(phenylethynyl)-pyridine (MPEP) and related compounds. These compounds do not interact with the orthosteric glutamate binding site but bind to an allosteric site in the seven transmembrane-spanning domain of GRM5 to inhibit coupling of the receptor to GTP binding proteins (Knoflach et al. (2001). Positive allosteric modulators of metabotropic glutamate 1 receptor: characterization, mechanism of action, and binding site. PNAS. 98(23): 13402-13407). These mGluR₅-selective negative allosteric modulators (NAMs) have had a major effect on understanding of the physiological roles of this receptor and have allowed studies that suggest that antagonists of mGluR₅ have potential as novel therapeutic agents (Rodriguez et al. (2010). Discovery of novel allosteric modulators of metabotropic glutamate receptor subtype 5 reveals chemical and functional diversity and in vivo activity in rat behavioral models of anxiolytic and antipsychotic activity. Mol. Pharmacol. 78(6): 1105-1123).

The inventors of the present disclosure discovered neuromodulatory peptides with novel structures and having binding capacity to the NAM site of GRM5 receptor. Anxiolytic- and antidepressant-like activity of these peptides was discovered to be comparable to ketamine and such SSRIs as fluvoxamine and fluoxetine. This was confirmed by experiments on zebrafish (Danio rerio) and rodents. Also, the inventors observed a prominent effect of the peptides on locomotion of animals in various behavioral paradigms. Further studies supported anxiolytic-like and antidepressant-like effects of RAHE (SEQ ID NO: 3) and KEDV (SEQ ID NO: 4) in Acute Foot Shock stress model. Electric foot shock is a complex stressor with both physical and emotional components, which has been employed as a tool to develop diverse animal models in the field of psychopharmacology. See Bali & Jaggi. Electric foot shock stress: a useful tool in neuropsychiatric studies. Rev Neurosci. 2015; 26(6):655-77. In embodiments, RAHE (SEQ ID NO: 3) and/or KEDV (SEQ ID NO: 4) are administered intranasally, or using other administration routes. In support of the hypothesis of negative allosteric modulation of GRMS by the studied peptides, a GRMS-luciferase reporter assay in HEK 293 cells, as well as a study of sodium glutamate-evoked [Ca²+] responses in CHO-mGluR5 cells, were carried out. The results are discussed in more detail in Examples below.

The inventors of the present disclosure have computationally created a set of peptides, wherein the peptides in the set were hypothesized to be GRMS negative allosteric modulator (NAM) peptides with the affinity to the NAM binding site. A three-dimensional docking algorithm was used to select more relevant peptides from the peptide sets. As a result, a family of tetrapeptides having novel sequences was identified. In some aspects, a number of tetrapeptides were computationally generated as potential drugs, and a subset of the tetrapeptides was tested. In some aspects, in vivo behavioral testing in zebrafish (Danio rerio) and in rodents has confirmed that four illustrative peptides, AGAS (SEQ ID NO: 1), DSGH (SEQ ID NO: 2), RAHE (SEQ ID NO: 3), KEDV (SEQ ID NO: 4), have anxiolytic-like and/or antidepressant-like activity comparable to ketamine and fluvoxamine and/or stimulating effect on locomotion comparable to caffein, as discussed in more detail below.

The inventors of the present disclosure designed and evaluated neuromodulatory peptides that were estimated to have binding capacity to the NAM site of GRMS receptor. In some aspects, the inventors of the present disclosure conducted the computational analysis and experiments in animal models as described herein, and, as a result, a group of tetrapeptides defined by a following general formula was identified: R₁R₂R₃R₄.

In some aspects, the R1 is an amino acid located in the NAM site of GRM5 receptor. The N-terminus of the peptide can be located in the NAM site. Alternatively, the C-terminus of the peptide can be located in the NAM site. In the following description herein, the peptide is defined as a sequence extending from the N-terminus to the C-terminus.

The inventors evaluated efficacy of the four illustrative peptides, AGAS (SEQ ID NO: 1), DSGH (SEQ ID NO: 2), RAHE (SEQ ID NO: 3), KEDV (SEQ ID NO: 4), as well as tested other (known) test substances, using zebrafish (Danio rerio), BALB/c mice and Sprague-Dawley rats as a model, as discussed in more details below in the Examples section. It has previously been observed that an increased anxiety of zebrafish is associated with an enhanced time spent in a dark compartment of a light/dark box (an increase in scototaxis—the desire to be in a dark shelter) and increased time spent at the bottom of the Novel Tank test. An increase in scototaxis was shown to be caused by a shift in the exploratory-hiding motivation balance towards the hiding. Maximino (2011) Pharmacological analysis of zebrafish (Danio rerio) scototaxis. Prog Neuropsychopharmacol Biol Psychiatry 35: 624-631; Nguyen (2014) Aquatic blues: Modeling depression and antidepressant action in zebrafish. Prog. Neuro-Psychopharmacology Biol. Psychiatry 55:26-39. Thus, the Danio rerio is a suitable model for evaluating potential anxiolytic substances.

The findings by the inventors of the present disclosure are consistent with the published data on the subject, according to which Danio rerio behavior under the conditions of the Novel Tank, the Light/Dark Box is an adequate model for assessing anxious behavior, as well as evaluating the effects of antidepressant and anxiolytic drugs. Maximino (2014). Fingerprinting of Psychoactive Drugs in Zebrafish Anxiety-Like Behaviors. PLoS One. 9. P. e103943.

In some embodiments, a composition is provided that comprises a synthetic neuromodulatory peptide, that is defined by the general formula I:

R₁R₂R₃R₄ (I),

wherein at least one of R₁-R₄ is hydrophobic and at least one of R₁-R₄ is polar or charged; none of R₁-R₄ is selected from L, M, I, T, C, P, N, Q, F, Y, and W; and the peptide modulates the mGluR₅ receptor (GRM₅).

In some embodiments, R₁ is R or K. In some embodiments, R₁ is D or E. In some embodiments, R₁ is S. R₂ can be hydrophilic neutral or negatively charged hydrophilic. In some embodiments, R₂ is hydrophobic neutral. In some embodiments, R₂ is A, S, E, or D. In some embodiments, R₃ is G, H, S, or D. In some embodiments, R₄ is S, H, V or E.

In some embodiments, R₁ is D, R₂ is 5, R₃ is G, and R₄ is H. In some embodiments, R₁ is R, R₂ is A, R₃ is H, and R₄ is E. In some embodiments, R₁ is K, R₂ is E, R₃ is D, and R₄ is V. In some embodiments, R₁ is A, R₂ is G, R₃ is A, and R₄ is S.

In some embodiments, each of R₁, R₂, and R₃ is a hydrophobic, aliphatic amino acid; and R₄ is a polar and neutral of charge hydrophilic amino acid.

In some embodiments, R₁ is a polar and negatively charged hydrophilic amino acid; R₂ is a polar and neutral of charge hydrophilic amino acid; R₃ is a hydrophobic, aliphatic amino acid; and R₄ is an aromatic, polar and positively charged hydrophilic amino acid.

In some embodiments, R₁ is D; R₂ is S; R₃ is G, A, or V; and R₄ is H.

In some embodiments, R₁ is a polar and positively charged hydrophilic amino acid; R₂ is a hydrophobic, aliphatic amino acid; R₃ is an aromatic, polar and positively charged hydrophilic amino acid; and R₄ is a polar and negatively charged hydrophilic amino acid.

In some embodiments, R₁ is R or K; R₂ is G, A, or V; R₃ is H; and R₄ is D or E.

In some embodiments, R₁ is a polar and positively charged hydrophilic amino acid; R₂ is a polar and negatively charged hydrophilic amino acid; R₃ is a polar and negatively charged hydrophilic amino acid; and R₄ is a hydrophobic, aliphatic amino acid.

In some embodiments, R₁ is R or K; R₂ is D or E; R₃ is D or E; and R₄ is G, A, or V. In some embodiments, R₁ is selected from R, K, D, A, and E; R₂ is selected from A, S, G, D, and E; R₃ is selected from S, G, D, E, A, and H; and R₄ is selected from S, H, V, and E. In some embodiments, R₁ is D; R₂ is S; R₃ is G; and R₄ is H.

In some embodiments, a composition is provided that comprises a neuromodulatory peptide, that is defined by the general formula II:

R₁R₂R₃R₄ (II),

wherein (1) R₁ is selected from the amino acids that are non-hydrophobic and not aromatic; the amino acids that contain a full positive charge on a side chain; the amino acids that contain a full negative charge on a side chain; and the amino acids that are non-charged and contain no more than 5 atoms in the side chain; (2) R₂ is selected from the amino acids that are non-charged and containing no more than 5 atoms in a side chain, and the amino acids that contain a full negative charge on a side chain; (3) R₃ is selected from the amino acids that are non-charged and contain no more than 5 atoms in a side chain; the amino acids that contain a full negative charge on a side chain; and the amino acids that are aromatic non-hydrophobic; and (4) R₄ is selected from the amino acids that do not include W, Y, F, P, I.

In some embodiments, R₁ is selected from A, R, K, D, E, Q, N, S, T, C, and M; R₂ is selected from A, S, G, D, and E; R₃ is selected from S, A, G, D, E, and H; and R₄ is selected from S, H, V, and E.

In some embodiments, R₁ is selected from R, K, D, A, and E; R₂ is selected from A, S, G, D, and E; R₃ is selected from S, G, D, E, A, and H; and R₄ is selected from S, H, V, and E.

In some embodiments, R₁ is D, R₂ is S; R₃ is G, and R₄ is H. In some embodiments, R₁ is R, R₂ is A, R₃ is H, and R₄ is E. In some embodiments, R₁ is K, R₂ is E, R₃ is D, and R₄ is V. In some embodiments, R₁ is A, R₂ is G, R₃ is A, and R₄ is S.

In some embodiments, R₁ is R, K, D, E, S or A. In some embodiments, R₂ is 5, A, G, or E. In some embodiments, R₃ is G, H, D or A.

In some embodiments, a composition is provided that comprises a synthetic neuromodulatory peptide, that is defined by the general formula III:

R₁R₂R₃R₄ (III),

wherein (1) R₁ is selected from the following residues: a non-hydrophobic amino acid which contain a system of connected p-orbitals for stacking interactions, but not an aromatic amino acid or amino acid containing a full positive charge on a side chain or an amino acid containing a full negative charge on a side chain or a non-charged amino acid, containing not more than 5 atoms in the side chain; (2) R₂ is selected from those amino acid residues which are non-charged, containing not more than 5 atoms in a side chain or containing a full negative charge on a side chain; (3) R₃ is selected from those amino acid residues which are non-charged, containing not more than 5 atoms in the side chain or containing a full negative charge on a side chain or aromatic non-hydrophobic; and (4) R₄ is selected from any amino acid residues except for hydrophobic aromatic or branched-chain hydrophobic, amino acid residues, with the proviso that V is optionally included in R4.

In embodiments, R₁ is selected from positively charged R or K, negatively charged D or E, or S or A. In embodiments, R₂ is selected from A, S, G, and D, E. In embodiments, R₃ is selected from A, G, H, S, D. In some embodiments, R₄ does not include W, Y, F, P, I.

In some embodiments, the synthetic neuromodulatory peptide consists of amino acids A, G, A, and S. In some embodiments, the synthetic neuromodulatory peptide consists of amino acids D, S, G, and H. In some embodiments, the synthetic neuromodulatory peptide consists of amino acids K, E, D, and V. In some embodiments, the synthetic neuromodulatory peptide consists of amino acids R, A, H, and E.

In some embodiments, a composition is provided that comprises a synthetic neuromodulatory peptide, that is defined by the general formula IV:

R₁R₂R₃R₄ (IV)

wherein R₁ is a non-hydrophobic amino acid; R₂ is a non-charged or hydrophilic amino acid; R₃ is a polar hydrophilic or aliphatic neutral amino acid; and R₄ is selected from the amino acids that do not include W, Y, F, P, I.

In some embodiments, R₁ is R or K. In some embodiments, R₁ is D or E. In some embodiments, R₁ is S. In some embodiments, R₁ is selected from the amino acids that do not include F, Y, W, and H.

In some embodiments, R₂ is hydrophilic neutral. In some embodiments, R₂ is negatively charged hydrophilic. In some embodiments, R₂ is hydrophobic neutral. In some embodiments, R₂ is A, S, E, or D.

In some embodiments, R₃ is G, H, S, or D.

In some embodiments, R₄ is S, H, V or E.

In some embodiments, R₁ is D, R₂ is S, R₃ is G, and R₄ is H. In some embodiments, R₁ is R, R₂ is A, R₃ is H, and R₄ is E. In some embodiments, R₁ is K, R₂ is E, R₃ is D, and R₄ is V.

In some embodiments, R₁ is selected from R, K, D, E, Q, N, S, T, C, and M; R₂ is selected from A, S, G, D, and E; R₃ is selected from S, G, D, E, and H; and R₄ is selected from S, H, V, and E.

In embodiments, the peptide is a tetrapeptide and the first, second and third amino acid residue is a hydrophobic, aliphatic amino acid such as glycine (G), alanine (A), leucine (L), isoleucine (I), methionine (M), or valine (V) while the fourth amino acid residue is a polar and neutral of charge hydrophilic amino acid, such as asparagine (N), glutamine (Q), serine (S), threonine (T), proline (P), and cysteine (C)

In embodiments, the peptide is a tetrapeptide and the first amino acid residue is a polar and negatively charged hydrophilic amino acid, such as aspartate (D) or glutamate (E), the second amino acid residue is a polar and neutral of charge hydrophilic amino acid, such as asparagine (N), glutamine (Q), serine (S), threonine (T), proline (P), and cysteine (C), the third amino acid residue is a hydrophobic, aliphatic amino acid such as glycine (G), alanine (A), leucine (L), isoleucine (I), methionine (M), or valine (V), and the fourth amino acid residue is an aromatic, polar and positively charged hydrophilic amino acid, such as histidine (H).

In embodiments, the peptide is a tetrapeptide and the first amino acid residue is the first amino acid residue is a polar and positively charged hydrophilic amino acid, such as arginine (R) or lysine (K), the second amino acid residue is a hydrophobic, aliphatic amino acid such as glycine (G), alanine (A), leucine (L), isoleucine (I), methionine (M), or valine (V), the third amino acid residue is an aromatic, polar and positively charged hydrophilic amino acid, such as histidine (H), and the fourth amino acid residue is a polar and negatively charged hydrophilic amino acid, such as aspartate (D) or glutamate (E).

In embodiments, the peptide is a tetrapeptide and the first amino acid residue is the first amino acid residue is a polar and positively charged hydrophilic amino acid, such as arginine (R) or lysine (K), the second amino acid residue is a polar and negatively charged hydrophilic amino acid, such as aspartate (D) or glutamate (E), the third amino acid residue is a polar and negatively charged hydrophilic amino acid, such as aspartate (D) or glutamate (E), and the fourth amino acid residue is a hydrophobic, aliphatic amino acid such as glycine (G), alanine (A), leucine (L), isoleucine (I), methionine (M), or valine (V).

The neuromodulatory peptide in accordance with the present disclosure can be in the form of a pharmaceutical composition. The composition can be administered to a subject in need of a treatment, e.g., a subject diagnosed with a disorder manifesting in depression and/or anxiety and/or motor impairments.

In some embodiments, the neuromodulatory peptide consists of amino acids that do not include proline.

In some embodiments, the peptide, or more than one peptide, in accordance with the present disclosure can be included as an active ingredient in a foodstuff. In these embodiments, the peptide can be included in a composition that is a food preparation. The food composition can include any non-active ingredients. Furthermore, the food composition can include, in addition to the peptide(s) in accordance with the present disclosure, other active ingredients that do not affect the effectiveness of the peptide.

In some embodiments, a peptide in accordance with the present disclosure is an active ingredient of the composition. In other embodiments, the active ingredient of the composition is an analog of the peptide, which can be an N-terminal modified analog or a C-terminal modified analog.

In some embodiments, the peptide in accordance with the present disclosure is optionally chemically modified. In some embodiments, the chemical modification is selected from amidation, methylation, and acetylation of one or more of R₁, R₂, R₃, and R₄, as described herein for Formulas I, II, III, or IV. In other embodiments, other various types of peptide backbone and/or side chain modifications can be performed. In some embodiments, the chemical modification is selected from addition of formyl, pyroglutamyl (pGlu), a fatty acid, urea, carbamate, sulfonamide, alkylamine, or any combination thereof, to one or more of R₁, R₂, R₃, and R₄, as described herein for Formulas I, II, III, or IV.

For example, in some embodiments, the peptide can be a “pseudo-peptide” where the regular peptide bond (CO—NH) is replaced with one of an isosteric or isoelectronic analog. For example, the reduced amide (CH2-NH) can be isosterically introduced into the peptide. In some embodiments, the peptide can be made in the form of azapeptide, where α-Carbon of the peptide backbone is replaced with nitrogen (without changing the amino acids residues). As a further example of a chemical modification, the synthetic neuromodulatory peptide in accordance with the present disclosure can be a retro-inverso peptide where a D-amino acid is used in a reversed sequence. As yet another example, in some embodiments, the synthetic neuromodulatory peptide in accordance with the present disclosure can be peptidomimetic having its side chains appended to the nitrogen atom of the peptide backbone, rather than to the α-carbons. In this way, the synthetic neuromodulatory peptide can be, in some embodiments, a peptoid, or poly-N-substituted glycine.

In some embodiments, the synthetic neuromodulatory peptide can be optionally modified by incorporating non-natural amino acids into certain positions in the peptide. Non-limiting examples of the non-natural amino acids include D-amino acids, N-methylated (or N-alkylated) amino acids, alpha-substituted alpha-amino acids, beta-substituted alpha-amino acids, beta-amino acids, and gamma-amino acids.

In some embodiments, the synthetic neuromodulatory peptide can be modified by cyclization of the peptide. In some embodiments, the synthetic neuromodulatory peptide can be modified such that the peptide is a beta-turn mimetics peptide. In some embodiments, phenylalanine (F) in the peptide, if present, can be replaced with nitro-, amino-, fluoro-phenylalanine, or other inhibitors of proteases.

In some embodiments, the composition in accordance with the present disclosure comprises a pharmaceutically acceptable carrier.

In some embodiments, the composition in accordance with the present disclosure further comprises a delivery vehicle. The delivery vehicle can be selected from a liposome, a nanoparticle, and a polysaccharide. In some embodiments, the polysaccharide can be selected from cyclodextrin, chitosan, cellulose, and alginate.

The composition in accordance with the present disclosure can be formulated for various routes of administration. Non-limiting examples of routes of administration include inhalation, intranasal, oral, intravenous, intramuscular, and subcutaneous.

In some embodiments, the composition is formulated for intranasal administration. The composition formulated for intranasal administration can include at least one inhibitor of nasal mucosa proteases. Non-limiting examples of the inhibitors include one or more compounds selected from bestatine, comostate amylase, leupeptin, aprotinin, bacitracin, amastatine, boroleucine, puromycin, a bile salt, and a fusidic acid (e.g., disodium ethylenediaminetetraacetate). The intranasal delivery is a noninvasive route of administration for the therapeutic peptides and provides an alternative to intravenous or subcutaneous injections.

In some embodiments, the composition is formulated for administration by inhalation. In some embodiments, the composition formulated for administration by inhalation can be administered using an intranasal device. The intranasal device can be, for example, a dry powder intranasal device configured to deliver a therapeutic agent to a subject in the form of a dry powder. In some embodiments, the intranasal device can be configured for use outside of a clinical setting, such that a therapeutic agent can be self-administered by a subject.

In some embodiments, the composition is formulated for intravenous administration.

In some embodiments, the composition is formulated for oral administration.

In some embodiments, the peptide modulates the mGluR₅ receptor (GRM5).

In some embodiments, a pharmaceutical composition is provided in accordance with any of the embodiments or any combination of the embodiments described herein, the pharmaceutical composition comprising a therapeutically effective amount of the composition and at least one pharmaceutically acceptable carrier, diluent, or excipient.

In some embodiments, a method for modulating mGluR₅ (GRM5) receptor in a cell is provided. The method comprises contacting the cell with the composition in accordance with any of the embodiments or any combination of the embodiments described herein.

In some embodiments, a method for treating a mood disorder in a patient in need thereof is provided, the method comprising administering a therapeutically effective amount of the composition in accordance with any of the embodiments described herein to a patient in need thereof. In some embodiments, a method for treating a mood disorder and movement disorder in a patient in need thereof is provided. The method comprises administering a therapeutically effective amount of the composition in accordance with any of the embodiments or any combination of the embodiments described herein to a patient in need thereof. The mood disorder can be depression. In some embodiments, the depression is selected from major depressive disorder, dysthymia, breakthrough depression, treatment-refractory depression, and depression associated with Parkinson's disease, depression associated with post-traumatic stress disorder, post-partum depression, and bipolar depression. In some embodiments, the mood disorder can be a stress-related disorder.

In some embodiments, the mood disorder is an anxiety disorder. The anxiety disorder can be selected from generalized anxiety disorder, social anxiety disorder, and panic disorder. In some embodiments, the mood disorder is schizophrenia. In some embodiments, the mood disorder is a post-traumatic stress disorder.

In some embodiments, the mood disorder is schizophrenia. In some embodiments, the mood disorder is a panic disorder. In some embodiments, the mood disorder is stress-related disorder.

In some embodiments, the movement disorder is a hypokinetic movement disorder or a hyperkinetic movement disorder. In embodiments, the hypokinetic movement disorder is selected from Parkinson's disease (primary or idiopathic Parkinsonism), secondary Parkinsonism, Parkinson plus syndromes, Hallevorden-Spatz disease, progressive supranuclear ophthalmoplegia, and striatonigral deneneration. In embodiments, the hyperkinetic movement disorder is selected from dystonia, drug induced dystonia, idiopathic familial dystonia, idiopathic nonfamilial dystonia, spasmodic torticollis, ideopathic orofacial dystonia, blepharospasm, essential tremor, drug induced tremor, myoclonus, opsoclonus, chorea, drug induced chorea, rheumatic chorea (Sydenham's chorea), Huntington's chorea, ballismus, hemiballismus, athetosis, dyskinesia, tardive dyskinesia, levodopa-induced dyskinesia, tic disorders, Tourette's syndrome, stereotypic movement disorder, paroxysmal nocturnal limb movement, restless leg syndrome, stiff-person syndrome, and cerebral palsy. In some embodiments, Parkinson's disease comprises primary Parkinson's disease or idiopathic Parkinson's disease, secondary Parkinson's disease or Parkinson plus syndrome. In some embodiments, the movement disorder can be dystonia, essential tremor, Huntington's chorea, Tourette's syndrome, stereotypic movement disorder. In some embodiments, the movement disorder can be attention deficit hyperactivity disorder. In some embodiments, the movement disorder comprises catatonia.

In some embodiments, the present invention provides a method of treating ADHD in a patient in need thereof comprising administering an effective amount of a composition comprising a synthetic neuromodulatory peptide. In an embodiment, the synthetic neuromodulatory peptide is administered in combination with an additional therapeutic agent.

In some embodiments, the present invention includes treatment of ADHD and/or the symptoms thereof. ADHD is a disorder characterized by, for example, inattentiveness, over-activity, impulsivity, or a combination. Decreased phasic dopamine release is believed, without wishing to be bound by theory, to be an important deficit in ADHD.

Accordingly, the methods and compositions of the present invention are useful for treatment of ADHD and/or the symptoms thereof. Any type of ADHD may be treated using methods and compositions of the invention, including but not limited to, combined type ADHD, predominantly inattentive type ADHD, and predominantly hyperactive-impulsive type ADHD.

In some embodiments, the present invention is useful for treatment of both ADHD and depression in the same subject. In some embodiments, the present invention provides a method for treating ADHD by administering an effective amount of a composition comprising a synthetic neuromodulatory peptide to a patient in need thereof. The patient may also receive pre-existent and/or combination therapy that comprises one or more of the additional therapeutic agents described herein.

The efficacy of treating ADHD using methods and compositions of the present invention may be assessed by various methods. For example, efficacy may be assessed using ADHD rating scales as described, for example, in Madaan et al., (2008) (CNS Drugs, 22(4):275-90), the entire contents of which are hereby incorporated by reference. Illustrative ADHD scales include, for example, the adult ADHD self-report scale (ASRS), the ADHD Behavior Checklist/ADHD Rating Scale, and the ADHD Investigator Symptom Rating Scale (AISRS).

In some embodiments, the present invention provides a method of treating schizophrenia in a patient in need thereof comprising administering an effective amount of a composition comprising a synthetic neuromodulatory peptide. In an embodiment, the synthetic neuromodulatory peptide is administered in combination with an additional therapeutic agent.

In some embodiments, the present invention includes treatment of schizophrenia and/or the symptoms thereof. Schizophrenia, characterized by a spectrum of psychopathology, refers to a chronic debilitating disorder that can be divided into subtypes based on the clinical picture. A paranoid subtype of schizophrenia is characterized by the presence of delusions or auditory hallucinations, a disorganized subtype of schizophrenia is manifested in disorganized speech and behavior, as well as flat or inappropriate affect. A feature of a catatonic subtype of schizophrenia is a psychomotor disturbance that may involve both motoric immobility as well as excessive motor activity. The catatonic schizophrenia may include stupor (a state close to unconsciousness) catalepsy (trance seizure with rigid body), waxy flexibility (limbs stay in the position another person puts them in), and mutism (lack of verbal response). A residual subtype of schizophrenia is characterized by a lack of prominent positive symptoms. Finally, undifferentiated schizophrenia is a classification used when a person exhibits behaviors of more than one other types of schizophrenia, including delusions, hallucinations, disorganized speech or behavior, and catatonic behavior.

Accordingly, the methods and compositions of the present invention are useful for treatment of schizophrenia and/or the symptoms thereof. Any type of schizophrenia may be treated using methods and compositions of the invention, including but not limited to, paranoid schizophrenia, catatonic schizophrenia, residual subtype of schizophrenia, and undifferentiated schizophrenia.

In some embodiments, the present invention is useful for treatment of both schizophrenia and depression in the same subject. In some embodiments, the present invention provides a method for treating schizophrenia by administering an effective amount of a composition comprising a synthetic neuromodulatory peptide to a patient in need thereof. The patient may also receive pre-existent and/or combination therapy that comprises one or more of the additional therapeutic agents described herein.

Patients with schizophrenia often have symptoms of other psychiatric disorders, including ADHD. Accordingly, in some embodiments, the present invention is useful for treatment of both ADHD and schizophrenia in the same subject. In some embodiments, the present invention provides a method for treating ADHD and schizophrenia by administering an effective amount of a composition comprising a synthetic neuromodulatory peptide to a patient in need thereof. The patient may also receive pre-existent and/or combination therapy that comprises one or more of the additional therapeutic agents described herein. Examples of additional therapeutic agents include stimulants, such as, for example, methylphenidate and amphetamines, though any other one or more additional therapeutic agents can be used for treatment of both ADHD and schizophrenia.

Non-limiting examples of therapeutic agents used for treatment of schizophrenia include chlorpromazine (THORAZINE), fluphenazine (PROLIXIN), haloperidol (HALDOL), perphenazine (TRILAFON), thioridazine (MELLARIL), thiothixene (NAVANE), trifluoperazine (STELAZINE), aripiprazole (ABILIFY), aripiprazole lauroxil (ARISTADA), asenapine (SAPHRIS), brexpiprazole (REXULTI), cariprazine (VRAYLAR), clozapine (CLOZARIL), iloperidone (FANAPT), lumateperone tosylate (CAPLYTA), lurasidone (LATUDA), olanzapine (ZYPREXA), paliperidone (INVEGA SUSTENNA), paliperidone palmitate (INVEGA TRINZA), quetiapine (SEROQUEL), risperidone (RISPERDAL), and ziprasidone (GEODON).

Non-limiting examples of therapeutic agents used for treatment of ADHD include dextroamphetamine (DEXEDRINE), dextroamphetamine (ZENZEDI), dextroamphetamine and amphetamine (ADDERALL), dexmethylphenidate (FOCALIN), methylphenidate (METHYLIN; RITALIN), amphetamine sulfate (DYANAVEL; EVEKEO), dextroamphetamine (DEXEDRINE SPANSULE), dextroamphetamine and amphetamine (ADDERALL; MYDAYIS), dexmethylphenidate (FOCALIN), lisdexamfetamine (VYVANSE), methylphenidate (APTENSIO; CONCERTA; COTEMPLA; DAYTRANA; METADATE; RITALIN; QUILLICHEW; QUILLIVANT), atomoxetine (STRATTERA), clonidine (CATAPRES; KAPVAY), guanfacine (INTUNIV; TENEX), bupropion (WELLBUTRIN), and desipramine (NORPRAMIN), imipramine (TOFRANIL), nortriptyline (AVENTYL; PAMELOR).

Any of the above or other suitable therapeutic agents can be used as additional therapeutic agents, in conjunction with the compositions described herein.

In some embodiments, the present invention provides a method of treating a bipolar disorder in a patient in need thereof comprising administering an effective amount of a composition comprising a synthetic neuromodulatory peptide. The bipolar disorder can be bipolar I disorder, bipolar II disorder, cyclothymic disorder, or it can be a bipolar disorder with symptoms not matching those of the above three types. In an embodiment, the synthetic neuromodulatory peptide is administered in combination with an additional therapeutic agent. In some embodiments, the present invention includes treatment of bipolar disorder and/or the symptoms thereof. Non-limiting examples of therapeutic agents used for treatment of bipolar disorder include carbamazepine (CARBATROL; EPITOL; EQUETRO; TEGRETOL), divalproex sodium (DEPAKOTE), lamotrigine (LAMICTAL), lithium, valproic acid (DEPAKENE), haloperidol (HALDOL), loxapine (LOXITANE; ADASUVE), risperidone (RISPERDAL), aripiprazole (ABILIFY), asenapine (SAPHRIS), cariprazine (VRAYLAR), lurasidone (LATUDA), olanzapine (ZYPREXA), quetiapine fumarate (SEROQUEL), and ziprasidone (GEODON).

In some embodiments, a method for treating a mood disorder and/or a movement disorder in accordance with any of the embodiments or any combination of the embodiments described herein is provided, the method further comprises administering an antidepressant. The antidepressant is optionally selected from the group consisting of serotonin reuptake inhibitors, selective norepinephrine reuptake inhibitors, combined action SSRI/SNRI, serotonin-2 antagonist/reuptake inhibitors, an antidepressant with alpha-2 antagonism plus serotonin-2 and serotonin-3 antagonism, an antidepressant with serotonin/norepinephrine/dopamine reuptake inhibition, an antidepressant with norepinephrine and dopamine reuptake inhibition, 5-HT-1alpha antagonist, 5-HT-1beta antagonist, 5-HT1A receptor agonists, 5-HT1A receptor agonists and antagonists, 5-HT2 receptor antagonists, viloxazine hydrochloride, dehydroepiandosterone, NMDA receptor antagonists, AMPA receptor potentiators, substance P antagonists/neurokinin-1 receptor antagonists, nonpeptide Substance P antagonist, neurokinin 2 antagonists, neurokinin 3 antagonists, corticotropin-releasing factor receptor antagonists, antiglucocorticoid medications, glucocorticoid receptor antagonists, cortisol blocking agents, nitric oxide synthesize inhibitors, inhibitors of phosphodiesterase, enkephalinase inhibitors, GABA-A receptor agonists, free radical trapping agents, atypical MAOI's, selective MAOI inhibitors, hormones, folinic acid, leucovorin, tramadol, and tryptophan in combination with an antipsychotic drug, wherein said antipsychotic drug is selected from the group consisting of an atypical antipsychotic drug, and a dopamine system stabilizer.

In some embodiments, a method for treating mood and movement disorders in accordance with any of the embodiments or any combination of the embodiments described herein further comprises administering an additional depression treatment comprising one or more additional agents.

In some embodiments, a method for treating a movement disorder in accordance with any of the embodiments or any combination of the embodiments described herein is provided, the method further comprising administering an additional anxiety treatment for a movement disorder.

In some embodiments, the present disclosure provides compositions and methods in accordance with any of the described embodiments that further comprise an additional agent and methods of administering the additional agent to a subject. In some embodiments, the invention pertains to co-administration and/or co-formulation. Any of the compositions described herein may be co-formulated and/or co-administered with one or more suitable agents.

In some embodiments, the one or more additional agents comprise an agent selected from one or more of CYMBALTA oral, LEXAPRO oral, EFFEXOR XR oral, ZOLOFT oral, CELEXA oral, TRAZODONE oral, PROZAC oral, WELLBUTRIN XL oral, CITALOPRAM oral, PRISTIQ oral, AMITRIPTYLINE oral, SAVELLA oral, VIIBRYD oral, PAXIL CR oral, WELLBUTRIN oral, PAXIL oral, SERTRALINE oral, REMERON oral, NORTRIPTYLINE oral, VENLAFAXINE oral, FLUOXETINE oral, BUPROPION HCL oral, MIRTAZAPINE oral, RITALIN oral, PAROXETINE oral, WELLBUTRIN SR oral, DOXEPIN oral, METHYLPHENIDATE oral, SYMBYAX oral, ESCITALOPRAM OXALATE oral, PAMELOR oral, IMIPRAMINE oral, BRINTELLIX oral, DULOXETINE oral, NARDIL oral, FETZIMA oral, EMSAM TRANSDERMAL, PARNATE oral, PEXEVA oral, BRISDELLE oral, CLOMIPRAMINE oral, ANAFRANIL oral, TOFRANIL oral, FLUVOXAMINE oral, ZYBAN oral, DESIPRAMINE oral, SARAFEM oral, PROZAC WEEKLY oral, APLENZIN oral, METHYLIN oral, NEFAZODONE oral, QUILLIVANT XR oral, TOFRANIL-PM oral, NORPRAMIN oral, REMERON SOLTAB oral, BUPROPION HBR oral, OLEPTRO ER oral, DESVENLAFAXINE SUCCINATE oral, BUPROBAN oral, IMIPRAMINE PAMOATE oral, VILAZODONE oral, MILNACIPRAN oral, PAROXETINE MESYLATE oral, SURMONTIL oral, MAPROTILINE oral, PROTRIPTYLINE oral, PHENELZINE oral, MARPLAN oral, OLANZAPINE-FLUOXETINE oral, TRANYLCYPROMINE oral, SELEGILINE TRANSDERMAL, AMOXAPINE oral, FORFIVO XL oral, ISOCARBOXAZID oral, DESVENLAFAXINE oral, KHEDEZLA oral, LEVOMILNACIPRAN oral, VORTIOXETINE oral, and DESVENLAFAXINE FUMARATE oral.

In some embodiments, inclusive, without limitation of those involving movement disorders, the additional agent is one or more of LEVODOPA, CARBIDOPA, SAFINAMIDE, PRAMIPEXOLE, ROTIGOTINE, ROPINIROLE, AMANTADINEM, BENZTROPINE, TRIHEXYPHENIDYL, SELEGILINE, RASAGILINE, ENTACAPONE, TOLCAPONE, DIAZEPAM, CLONAZEPAM, BACLOFEN, TRIHEXYPHENIDYL, BENZTROPINE, ETHOPROPAZINE, LORAZEPAM, BROMOCRIPTINE, TETRABENAZINE, PROPRANOLOL, PRIMIDONE, FLUPHENAZINE, HALOPERIDOL, RISPERIDONE, PIMOZIDE, ZIPRASIDONE, FLUPHENAZINE, AMPHETAMINE, METHYLPHENIDATE, DEXMETHYLPHENIDATE, METHYLPHENIDATE, ATOMOXETINE HYDROCHLORIDE, and LISDEXAMFETAMINE DIMESYLATE.

In some embodiments, the one or more additional agents comprise an agent selected from one or more of chlorpromazine (THORAZINE), fluphenazine (PROLIXIN), haloperidol (HALDOL), perphenazine (TRILAFON), thioridazine (MELLARIL), thiothixene (NAVANE), trifluoperazine (STELAZINE), aripiprazole (ABILIFY), aripiprazole lauroxil (ARISTADA), asenapine (SAPHRIS), brexpiprazole (REXULTI), cariprazine (VRAYLAR), clozapine (CLOZARIL), iloperidone (FANAPT), lumateperone tosylate (CAPLYTA), lurasidone (LATUDA), olanzapine (ZYPREXA), paliperidone (INVEGA SUSTENNA), paliperidone palmitate (INVEGA TRINZA), quetiapine (SEROQUEL), risperidone (RISPERDAL), and ziprasidone (GEODON).

In some embodiments, the one or more additional agents comprise an agent selected from one or more of dextroamphetamine (DEXEDRINE), dextroamphetamine (ZENZEDI), dextroamphetamine and amphetamine (ADDERALL), dexmethylphenidate (FOCALIN), methylphenidate (METHYLIN; RITALIN), amphetamine sulfate (DYANAVEL; EVEKEO), dextroamphetamine (DEXEDRINE SPANSULE), dextroamphetamine and amphetamine (ADDERALL; MYDAYIS), dexmethylphenidate (FOCALIN), lisdexamfetamine (VYVANSE), methylphenidate (APTENSIO; CONCERTA; COTEMPLA; DAYTRANA; METADATE; RITALIN; QUILLICHEW; QUILLIVANT), atomoxetine (STRATTERA), clonidine (CATAPRES; KAPVAY), guanfacine (INTUNIV; TENEX), bupropion (WELLBUTRIN), desipramine (NORPRAMIN), imipramine (TOFRANIL), and nortriptyline (AVENTYL; PAMELOR).

In some embodiments, the one or more additional agents comprise an agent selected from one or more of carbamazepine (CARBATROL; EPITOL; EQUETRO; TEGRETOL), divalproex sodium (DEPAKOTE), lamotrigine (LAMICTAL), lithium, valproic acid (DEPAKENE), haloperidol (HALDOL), loxapine (LOXITANE; ADASUVE), risperidone (RISPERDAL), aripiprazole (ABILIFY), asenapine (SAPHRIS), cariprazine (VRAYLAR), lurasidone (LATUDA), olanzapine (ZYPREXA), quetiapine fumarate (SEROQUEL), and ziprasidone (GEODON).

In some embodiments, the additional agent may be conjugated to the peptides in accordance with the present disclosure.

In some embodiments, a method for treating a mood disorder and movement disorder (which can be present in a mood disorder), in accordance with any of the embodiments or any combination of the embodiments described herein is provided, the method further comprising administering an additional anti-anxiety treatment optionally selected from one or more of benzodiazepines selected from alprazolam (XANAX), clonazepam (KLONOPIN), diazepam (VALIUM), lorazepam (ATIVAN), oxazepam (SERAX), and chlordiazepoxide (librium); beta blockers selected from propranolol (INDERAL) and atenolol (TENORMIN); tricyclic antidepressants selected from imipramine (TOFRANIL), desipramine (NORPRAMIN, PERTOFRANE), nortriptyline (AVENTYL or PAMELOR), amitriptyline (ELAVIL), doxepin (SINEQUAN or ADAPIN), clomipramine (ANAFRANIL); monoamine oxidase inhibitors (MAOIs) selected from phenelzine (NARDIL), tranylcypromine (PARNATE); selective serotonin reuptake inhibitors (SSRIs) selected from fluoxetine (PROZAC), fluvoxamine (LUVOX), sertraline (ZOLOFT), paroxetine (PAXIL), escitalopram oxalate (LEXAPRO), citalopram (CELEXA); serotonin-norepinephrine reuptake inhibitors (SNRIs) selected from venlafaxine (EFFEXOR), venlafaxine extended release (EFFEXOR XR) and duloxetine (CYMBALTA); mild tranquilizers such as buspirone (BUSPAR); and anticonvulsants selected from valproate (DEPAKOTE), pregabalin (LYRICA), and gabapentin (NEURONTIN).

In some embodiments, a method for treating a neurodegenerative disorder in a patient in need thereof is provided, the method comprising administering a therapeutically effective amount of the composition in accordance with any of the embodiments described herein, or combinations thereof. The neurodegenerative disorder can be, for example, Parkinson's disease or Alzheimer's disease.

In some embodiments, a peptide (e.g., KEDV (SEQ ID NO: 4) and/or RAHE (SEQ ID NO: 3)) or a composition comprising the peptide in accordance with embodiments of the present disclosure, is used as a psychostimulant. A psychostimulant is an agent (e.g., a drug) having mood-enhancing and stimulant properties, it produces a temporary increase in psychomotor activity, increased alertness, or a temporary improvement in physical functions. A psychostimulant can also be negatively defined as a substance other than a depressant or a hallucinogenic substance. Favrod-Coune & Broers. Pharmaceuticals (Basel, Switzerland) vol. 3,7 2333-2361. 22 Jul. 2010, doi:10.3390/ph3072333. Psychostimulants are typically used to treat attention deficit disorder and sometimes depression, and are often used as illicit substances. Caffeine is the most consumed socially acceptable stimulant. Favrod-Coune & Broers (2010).

In embodiments, the peptide (e.g., KEDV (SEQ ID NO: 4) and/or RAHE (SEQ ID NO: 3)) is administered to a patient in need thereof. In some embodiments, a mixture of KEDV (SEQ ID NO: 4) and RAHE (SEQ ID NO: 3) can be administered. In some embodiments, administration of the peptide results in a decrease in the expression of the Kcna1 gene. The KCNA1 gene belongs to a family of genes that provide instructions for making potassium channels, and it provides instructions for making one part (the alpha subunit) of potassium voltage-gated channel subfamily A member 1 (Kv1.1, also denoted as Kv1.1). The Kv1.1 protein functions as a potassium selective channel through which the potassium ion may pass in consensus with the electrochemical gradient. In embodiments, the peptide, such as, without limitation, KEDV (SEQ ID NO: 4) and/or RAHE (SEQ ID NO: 3), is administered intranasally.

In embodiments, the present compositions may be fused to other moieties, e.g., an additional agent or a moiety to extend half-life in vivo. Apart from increasing stability, such moieties may also increase solubility of the molecule they are fused to. A moiety that increases solubility (e.g., prevents aggregation) may provide easier handling of the compositions, and particularly improve stability and shelf-life. A well-known example of such moiety is PEG (polyethylene glycol). This moiety is particularly envisaged, as it can be used as linker as well as solubilizing moiety. Other examples include peptides and proteins or protein domains, or even whole proteins (e.g., GFP). In this regard, it should be noted that, like PEG, one moiety can have different functions or effects. For instance, a flag tag (DYKDDDDK (SEQ ID NO: 5)) is a peptide moiety that can be used as a label, but due to its charge density, it will also enhance solubilization. PEGylation has already often been demonstrated to increase solubility of biopharmaceuticals (e.g., Veronese and Mero (2008) The impact of PEGylation on biological therapies, BioDrugs. 22(5)315-29). Adding a peptide, polypeptide, protein or protein domain tag to a molecule of interest has been extensively described in the art. Examples include, but are not limited to, peptides derived from synuclein (e.g., Park et al., Protein Eng. Des. Sel. 2004; 17:251-260), SET (solubility enhancing tag, Zhang et al., Protein Expr Purif 2004; 36:207-216), thioredoxin (TRX), Glutathione-S-transferase (GST), Maltose-binding protein (MBP), N-Utilization substance (NusA), small ubiquitin-like modifier (SUMO), ubiquitin (Ub), disulfide bond C (DsbC), Seventeen kilodalton protein (Skp), Phage T7 protein kinase fragment (T7PK), Protein G BI domain, Protein A IgG ZZ repeat domain, and bacterial immunoglobulin binding domains (Hutt et al. (2012) J Biol Chem.; 287(7):4462-9). The nature of the tag depends on the application, as can be determined by the skilled person. For instance, for transgenic expression of the molecules described herein, it may be envisaged to fuse the molecules to a larger domain to prevent premature degradation by the cellular machinery. Other applications may envisage fusion to a smaller solubilization tag (e.g., less than 30 amino acids, or less than 20 amino acids, or even less than 10 amino acids) in order not to alter the properties of the molecules too much. Additional chemical modifications can include addition of formyl, pyroglutamyl (pGlu), one or more fatty acids, urea, carbamate, sulfonamide, alkylamine, or any combination thereof.

Apart from extending half-life, the present compositions (e.g., one or more peptides in accordance with embodiments of the present disclosure) may be fused to moieties that alter other or additional pharmacokinetic and pharmacodynamic properties. For instance, it is known that fusion with albumin (e.g., human serum albumin), albumin-binding domain or a synthetic albumin-binding peptide improves pharmacokinetics and pharmacodynamics of different therapeutic proteins (Langenheim and Chen, Endocrinol.; 203(3):375-87, 2009). Another moiety that is often used is a fragment crystallizable region (Fc) of an antibody. The nature of these moieties can be determined by the person skilled in the art depending on the application.

In some embodiments, the peptides of the present disclosure can be administered as the sole pharmaceutical agent or in combination with one or more other pharmaceutical agents where the combination causes no unacceptable adverse effects.

The amount of the active ingredient to be administered in the treatment of one or more conditions can vary according to such considerations as the particular peptide and dosage unit employed, the mode of administration, the period of treatment, the age, weight, and sex of the patient treated, and the nature and extent of the condition treated. The composition in accordance with the present disclosure can be administered to a subject at the appropriate dose via a certain route.

In some embodiments, a dose of the peptide to be administered will generally range from about 0.001 mg/kg to about 200 mg/kg body weight, from about 0.01 mg/kg to about 100 mg/kg body weight, from about 0.01 mg/kg to about 50 mg/kg body weight, from about 0.01 mg/kg to about 40 mg/kg body weight, from about 0.01 mg/kg to about 30 mg/kg body weight, from about 0.01 mg/kg to about 20 mg/kg body weight, from about 0.01 mg/kg to about 5 mg/kg body weight, from about 0.01 mg/kg to about 10 mg/kg body weight, from about 0.1 mg/kg to about 10 mg/kg body weight, from about 0.1 mg/kg to about 20 mg/kg body weight, from about 0.1 mg/kg to about 30 mg/kg body weight, from about 0.1 mg/kg to about 40 mg/kg body weight, from about 0.1 mg/kg to about 50 mg/kg body weight. Clinically useful dosing schedules will range from one to three times a day dosing. A pharmaceutical composition with the neuromodulatory peptides described herein can also be administered as a single dose. Because of the safety and effectiveness of the composition, the single dose of the composition can be effective in alleviating depression- or anxiety-related symptoms. Treatment schedules can also be developed for a more prolonged treatment course. For example, in some embodiments, a pharmaceutical composition in accordance with embodiments of the present disclosure can be administered during more than one day, for instance, from 2 days to 60 days, or from 2 days to 50 days, or from 2 days to 40 days, or from 2 days for 30 days, and the daily dose can be within any of the above ranges. The administration for more than one day can be used for treatment of chronic symptoms or disorders, which can be any of various mental, behavioral, affective, neurotic, and emotional disorders, including depression, anxiety, and stress-related disorders.

A “subject” is a mammal, e.g., a human (e.g., a female or a male human), mouse, rat, guinea pig, dog, cat, horse, cow, pig, or non-human primate, such as a monkey, chimpanzee, baboon or rhesus, and the terms “subject” and “patient” are used interchangeably herein.

The invention further provides kits that can simplify the administration of any agent described herein. An illustrative kit of the invention comprises any composition described herein in unit dosage form. In one embodiment, the unit dosage form is a container, such as a pre-filled syringe, which can be sterile, containing any agent described herein and a pharmaceutically acceptable carrier, diluent, excipient, or vehicle. The kit can further comprise a label or printed instructions instructing the use of any agent described herein. The kit may also include a lid speculum, topical anesthetic, and a cleaning agent for the administration location. The kit can also further comprise one or more additional agents described herein. In one embodiment, the kit comprises a container containing an effective amount of a composition of the invention and an effective amount of another composition, such those described herein.

The present disclosure is further illustrated by the following non-limiting examples.

### EXAMPLES

### Example 1: Modeling of the Tetrapeptides Binding with the GRM5 (mGluR₅) Receptor

1.1. Study Objective:

Identification of the tetrapeptides with the maximum affinity for the binding site of negative allosteric modulators of the mGluR₅ receptor. Both computer-generated random tetrapeptides library, and experimentally derived peptides from casein and beta-lactoglobulin, were used as a source of tetrapeptides.

1.2. Results:

From the total pool of tetrapeptides, the ones containing cysteine were excluded which narrowed down the total amount of peptides to 130,321 peptides. For each peptide, docking to the Negative Allosteric Modulator site (NAM-site) of the GRM receptor was performed, generating up to 20 docking poses for each peptide. In total, 2 604 060 unique docking poses were generated. Then all docking poses were grouped and analyzed. Briefly, the approach was to measure how often an atom of a specific type (aromatic carbon atom, hydrogen bond donor/acceptor, etc.) enters a given region of the spatial density map. Each docking pose was evaluated based on this map and normalized to the number of atoms and residues. The conventional interactions (electrostatics, hydrogen bonding, etc.) were explicitly considered at the stage of docking.

During the previous studies of the limited subset of tetrapeptides, the structural features of the NAM docking site were identified. Due to the narrowness of the binding pocket, peptides with large side groups sterically were not be able to fit in it and grouped on the edge of the docking site. These peptides were excluded from further calculations in order to prevent the distortion of the probability model.

1.2.1. Computer-Generated Random Tetrapeptides Library

A threshold of 0.92 was used to cut off a sufficient number of the best findings with a good score. This gave 29 findings in total. The LOGO analysis of the top 29 sequences revealed that, while the first and the fourth positions are rather variable, the second position clearly prefers small amino acids: alanine, serine and glycine. The third position has a significant preference for glycine. For better statistical significance, the LOGO analysis was performed for the 1000 top scoring peptides, which revealed that the presence of alanine, serine and glycine at the second position is probably mandatory for the peptides that enter the site. The third position is even less variable for the top peptides, with a significant predominance of glycine.

There are only a few peptides with high score (>0.9) that did not fit the above rules. The peptides that do not contain serine and glycine in positions 2 and 3, are DAHK (SEQ ID NO: 6) with a score of 0.94, RAHE (SEQ ID NO: 3) with a score of 0.93, HAMR (SEQ ID NO: 7) with a score of 0.91, and HAHN (SEQ ID NO: 8) with a score of 0.91. The only peptide in the high score range that does not have alanine in the 2^{nd} position, is DTHK (SEQ ID NO: 9) with a score of 0.9.

The frequency of occurrence of individual tetrapeptides in the list of the best findings were estimated. The peptide DSGH (SEQ ID NO: 2) (0.93) has appeared twice, the rest of peptides occurred only once.

For a negative control, a variety of different peptides which didn't fit into the binding pocket or the peptides with a low score, could be used. For a precise and systematic evaluation, logos for all peptides, which poses didn't fit into the binding site, as well as for the peptides with pose scores from 0 to 0.1 were created. Thus, the peptides that have tyrosine, tryptophan or phenylalanine at the second position, and phenylalanine or tryptophan at the third position were suggested as a negative control. As an example, the following peptides could be taken: AYFE (SEQ ID NO: 10), GFWY (SEQ ID NO: 11), QWFA (SEQ ID NO: 12), HWWM (SEQ ID NO: 13).

1.2.3. Experimentally Derived Peptides from Casein and Beta-Lactoglobulin

The peptide sample from the casein hydrolysate yielded 274 peptides, 5480 poses in total. The peptide sample from casein (alpha, beta and kappa forms) consisted of 569 peptides, 10900 poses in total. There are no tetrapeptides from the experimental pool with the score higher than 0.92. The best score for hydrolysate peptides is 0.81, for casein peptides is 0.84. A new threshold of 0.74 for both subsamples was chosen, resulting in only 11 unique poses for the top hydrolysate peptides and 30 unique poses the top casein peptides. Regarding the frequency of occurrence, the peptide DAPS (SEQ ID NO: 14) (maximum score 0.76) has appeared twice, the rest of peptides were observed once. In the list of the top casein peptides, peptides ESRE (SEQ ID NO: 15) (0.84), ESTQ (SEQ ID NO: 16) (0.79), AAHA (SEQ ID NO: 17) (0.77) were found twice. The major hydrolysate peptides have rather low scores, with the best equal to 0.74.

The peptide sample from beta-lactoglobulin consists of 175 peptides, 3020 poses in total. If 0.74 threshold is used and the top of peptides released from beta-lactoglobulin are selected, as it was performed for the subsamples of hydrolysate and casein, then the following tetrapeptides will have the best scores.

1.3. Selection of Peptides for Testing

130,321 tetrapeptides were evaluated, as well as subsamples of tetrapeptides from milk hydrolysate, beta-lactoglobulin and casein, on their ability to bind with negative allosteric modulators of the GRMS receptor.

The representation of amino acids at individual positions of the top tetrapeptides were analyzed and observed a clear predisposition to glycine, alanine and serine at the second position and to glycine at the third position.

The analysis of the worst poses of peptides was also performed, and a predisposition to aromatic amino acids at the second and the third positions among their sequences was revealed. The following peptides: AYFE (SEQ ID NO: 10), GFWY (SEQ ID NO: 11), QWFA (SEQ ID NO: 12), HWWM (SEQ ID NO: 13) were suggested as a negative control.

The following peptides were selected for verification of the experiment:

AGAS (SEQ ID NO: 1) is the best combinatorial tetrapeptide according to the score values;

DSGH (SEQ ID NO: 2) is a combinatorial tetrapeptide with the best occurrence and the LOGO analysis;

RAHE (SEQ ID NO: 3) is a combinatorial peptide with a high score as well, but at the same time it is fundamentally different from the peptides selected above, because of the absence of the common amino acids G and S in the LOGO analysis;

KEDV (SEQ ID NO: 4) is the best (according to the score values) tetrapeptide among the major tetrapeptides from the composition of anxiolytic hydrolysate.

AYFE (SEQ ID NO: 10) is a negative control.

### Example 2: Screening for Neurotropic Activity of Selected Peptides in Zebrafish (Danio rerio)

The objective was to study the effects of psychoactive agents on behavior of zebrafish (Danio rerio) and to compare these results with the effects of rapid antidepressant ketamine.

2.1. Materials and Methods

2.1.1. Animal Maintenance

The zebrafish were kept in a flow-through ZebTEC Zebrafish housing system (manufactured by Tecniplast) at a temperature of 28° C., a pH of 6.8-7.5, an osmolality of 550-700 osmol/liter, with a light regimen of 12/12, and constant aeration. The zebrafish were fed a special diet twice a day. During the experiment, the fish were fed in the evening on the day prior to the experiment and in the evening on the day of the experiment after its completion.

2.1.2. Substances, Doses and Administration

Ketamine

A 10% ketamine solution was used as a reference substance.

AGAS

AGAS (SEQ ID NO: 1) is the best combinatorial tetrapeptide according to the score values.

DSGH

DSGH (SEQ ID NO: 2) is a combinatorial tetrapeptide with the best occurrence and the LOGO analysis.

RAHE

RAHE (SEQ ID NO: 3) is a combinatorial peptide with a high score as well, but at the same time it is fundamentally different from the peptides selected above, because of the absence of the common amino acids G and S in the LOGO analysis

KEDV

KEDV (SEQ ID NO: 4) is the best (according to the score values) tetrapeptide among the major tetrapeptides from the composition of anxiolytic hydrolysate.

AYFE

AYFE (SEQ ID NO: 10) was chosen as a negative control.

Administration

The tested substances were injected into the zebrafish intraperitoneally using an insulin syringe (0.5 ml, 30 g) 10 minutes before the test (15 min before for ketamine only, according to the literature data). The saline solution (0.9% NaCl) was used as a solvent. Anesthesia and immobilization of the animals were achieved by placing them in water at a temperature of 10° C. Control group fish received intraperitoneal injections of an equivalent volume of solvent, also after going through a pre-cooling procedure.

2.1.3. Equipment Setup

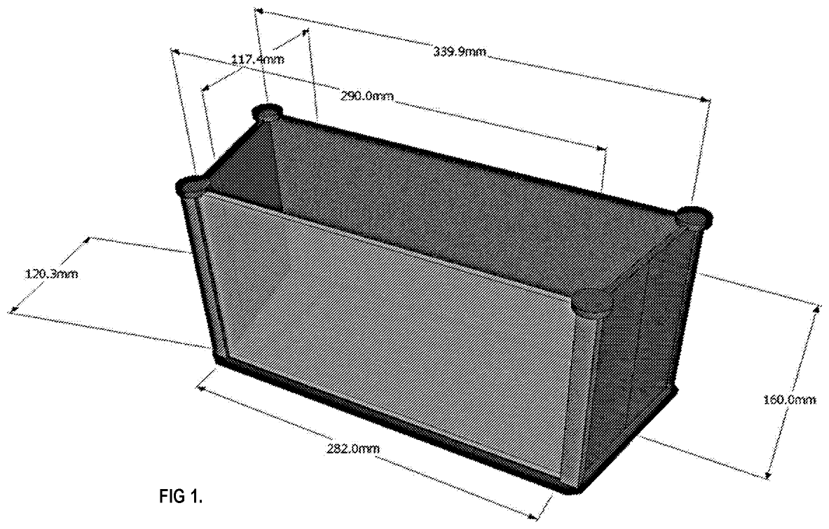

The Novel Tank test was conducted in a 4-liter trapezoid aquarium, the parameters of which are shown in FIG. 1. A base, back, and side walls of the aquarium were made of matte black plastic; the front panel (shorter wall) was made of transparent Plexiglas.

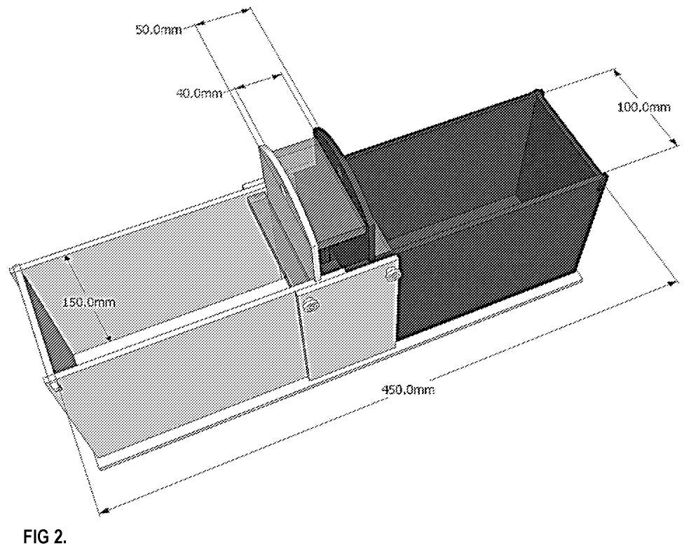

The setup for the Light/Dark preference test consisted of three main parts: a launch compartment, a light compartment made of white plastic, and a dark compartment made of black plastic. Installation parameters are shown in FIG. 2. The bright lighting in these tests was provided by a lamp on a stand (LED lamp PL, 11 W, light flux≈600 Lm, about 500 Lx directly above the water surface), which was attached to the upper part of the aquarium.

2.1.4. Behavioral Tests

2.1.4.1. The Novel Tank test

The Novel Tank (NT) test was performed as previously described (Maximino et al. (2013). Behavioral and neurochemical changes in the zebrafish leopard strain. G2B. 12(5): 576-582). A video recording (background shooting) was started 20-30 seconds before the zebrafish were placed in the test aquarium. The experimental zebrafish was placed in the tank using a net. The recording was being conducted for 5 minutes. The test was carried out using EthoVision XT software package (Noldus, Wageningen, Netherlands). The software registered the distance covered by the animal, its speed, the number of visits to the three conventional zones of the aquarium: “bottom,” “center,” and “middle” (lower, middle, and upper thirds of the aquarium, respectively), the time spent in these zones, and the latency of a visit to the middle and to the surface of the aquarium.

2.1.4.2. The Light/Dark Box test

The light/dark box (LDB) test was performed as previously described (Maximino et al. (2011). Pharmacological analysis of zebrafish (Danio rerio) scototaxis. Prog. Neuro-Psychopharmacol. Biol. Psychiatry. 35(2): 624-631). The zebrafish was placed in a light compartment of an LDB using a net, the camera was switched on simultaneously, and the behavior of the zebrafish was recorded for 5 minutes. The video was processed using RealTimer (OpenScience). During the processing of records, the residence time and the number of visits to the light and dark compartments of the test setup were recorded, as well as the latent period of visiting both compartments.

2.1.5. Data Analysis

Statistical analysis was performed by using the Statistical Package STATISTICA 10 and GraphPad Prism 6. Data were assessed for normality using of the Kolmogorov-Smirnov test to determine whether to use parametric or non-parametric statistical tests. For a pair comparison Student T-test or Mann-Whitney (M-W) U-test was used. Each treatment group had a corresponding control. Results are presented as mean±standard error of mean (SEM).

2.2. Evaluation of Effects of Test Substances on Behavior of Danio rerio

2.2.1. Ketamine

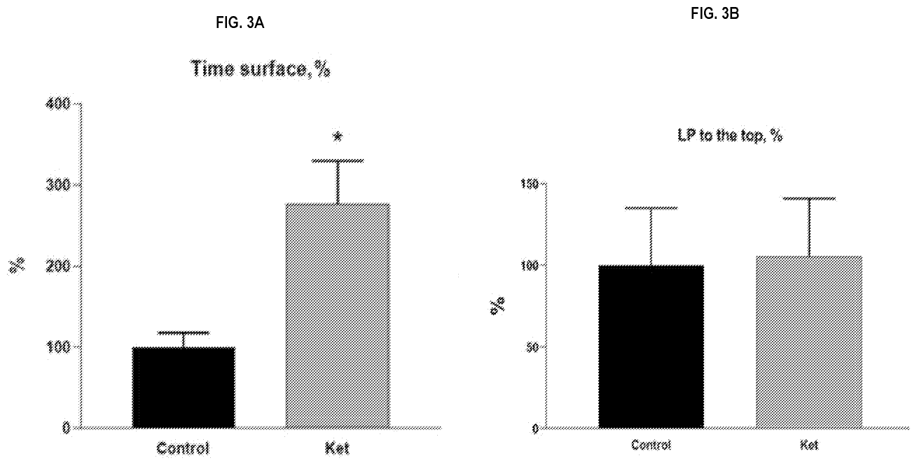

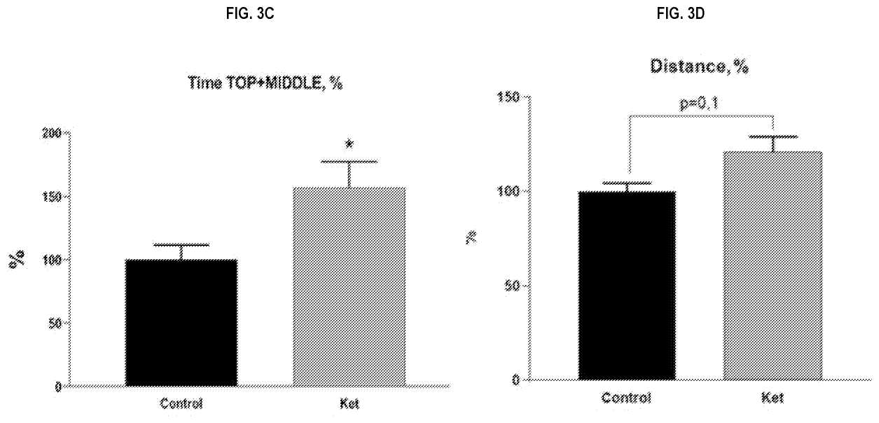

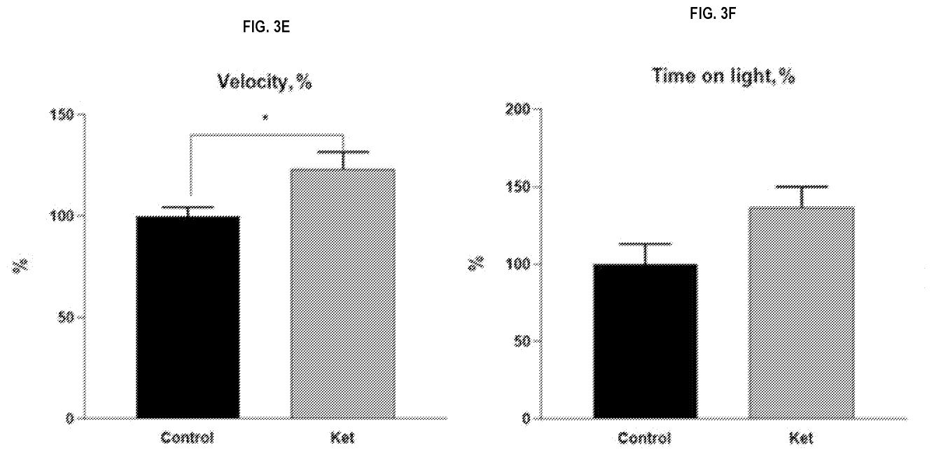

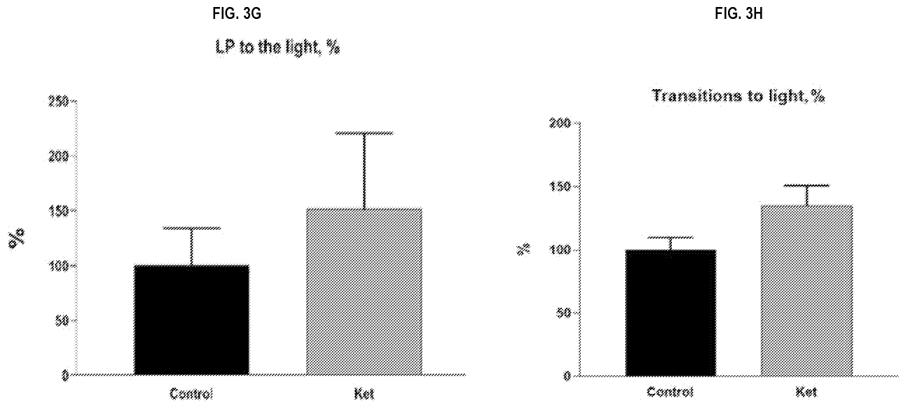

The most prominent effects of ketamine at a dose of 20 mg/kg were observed in the NT test. Those fish which received injection of ketamine showed a significant increase in time spent at the top of the tank and in the middle+top of the aquarium (FIGS. 3A, 3C). Moreover, ketamine treatment resulted in increased velocity of animals (FIG. 3E) as well as in tendency towards increased distance travelled (p<0.10 Mann-Whitney U-test, FIG. 3D). In the LDB test only trends towards significant differences were observed in animals after ketamine treatment (FIGS. 3F-3H).

These results may propose that ketamine at a dose of 20 mg/kg evokes anxiolytic-like responses in fish primarily in NT and LDB tests. At the same time, an increased velocity and a tendency towards distance travelled in NT and number of transitions in LDB tests may propose an increased exploratory activity or hyperactivity of animals which received ketamine. These results may propose that ketamine at a dose of 20 mg/kg evokes anxiolytic-like responses in fish in NT and LDB tests. At the same time, an increased velocity and a tendency towards distance travelled in NT and number of transitions in LDB tests may propose an increased exploratory activity or hyperactivity of animals which received ketamine.

2.2.2. AGAS

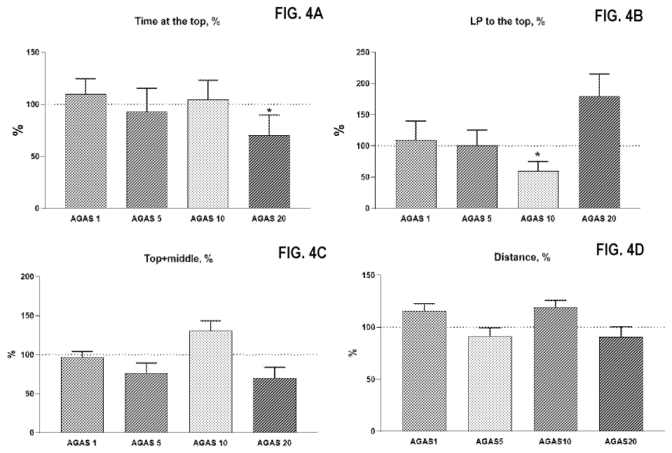

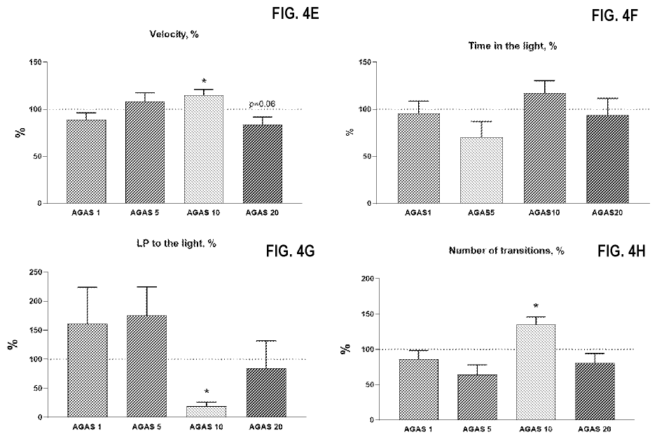

The AGAS (SEQ ID NO: 1) treatment at a dose of 1 and 5 mg/kg didn't show any prominent effects on behavior of animals in NT and LDB tests (FIGS. 4A-4H).

The fish injected with AGAS (SEQ ID NO: 1) at a dose of 10 mg/kg had greater velocity in NT than the animals from control group (FIG. 4E). Also, the peptide treatment resulted in decreased latency to reach the top of the tank (FIG. 4B) in fish. Also, a decreased latency to enter the light compartment was observed (FIG. 4G) and increased number of transitions between compartments of LDB (FIG. 4H) in animals injected with the AGAS (SEQ ID NO: 1) at a dose of 10 mg/kg. These results may indicate a positive neurotropic effect of the AGAS (SEQ ID NO: 1) tetrapeptide at a dose of 10 mg/kg on passive defensive behavior: the peptide administration promoted a shift towards exploratory activity, such as visits to different zones of the apparatuses, smaller latency to exit the “shelter” zone and increased locomotion. The inventors propose that AGAS (SEQ ID NO: 1) (10 mg/kg) may possess an anxiolytic-like activity in animals.

The AGAS (SEQ ID NO: 1) injection at a dose of 20 mg/kg resulted in a decreased time spent at the top of the aquarium and a trend towards significant reduction of velocity in the NT test (FIGS. 4A, 4E). However, there were no differences between experimental and control groups in LDB test. The inventors propose that AGAS (SEQ ID NO: 1) at a dose of 20 mg/kg has an anxiogenic-like or sedative effect. Thus, the dose-dependent study of AGAS (SEQ ID NO: 1) effects in NT and LDB tests revealed that:

AGAS (SEQ ID NO: 1) at a dose of 10 mg/kg has a prominent anxiolytic-like activity in Danio rerio.

AGAS (SEQ ID NO: 1) at a dose of 1 and 5 mg/kg did not affect the behavior of Danio rerio.

AGAS (SEQ ID NO: 1) at a dose of 20 mg/kg has an anxiogenic-like or sedative effect on Danio rerio.

2.2.3. DSGH

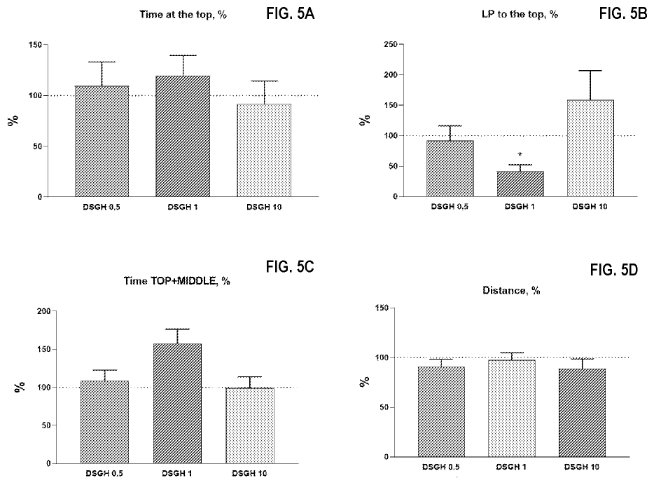

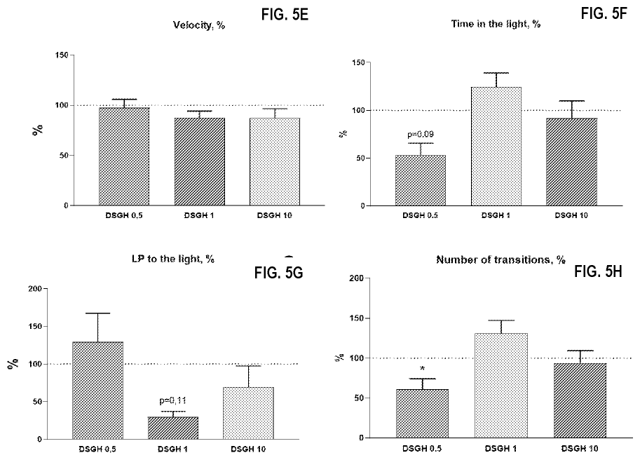

The DSGH (SEQ ID NO: 2) treatment at a dose of 0.5 mg/kg did not produced any behavioral effects in the NT test (FIGS. 5A-5E). A decreased time spent in the light compartment (trend towards significance p<0.1 according to M-W test) as well as decreased number of transitions to light in LDB (FIGS. 5F, 5H) was observed after DSGH (SEQ ID NO: 2) injection at a given dose, which may propose a shift towards defensive behavior in animals after DSGH (SEQ ID NO: 2) treatment.

The fish treated with DSGH (SEQ ID NO: 2) at a dose of 1 mg/kg showed a reduced latency to the top in the NT test (FIG. 5B) as well as a trend towards increased time spent in the light compartment and decreased latency to enter the light in the LDB test (p<0.10 Mann-Whitney U-test, FIG. 5F). The results may indicate a positive neurotropic activity of DSGH (SEQ ID NO: 2) at a dose of 1 mg/kg which resulted in increased exploratory activity in animals.

The injection of DSGH (SEQ ID NO: 2) at a dose of 10 mg/kg did not affect the behavioral parameters of fish in both experimental paradigms (FIGS. 5A-5H).

The results propose dose-dependent effects after DSGH (SEQ ID NO: 2) administration:

DSGH (SEQ ID NO: 2) at a dose of 0.5 mg/kg showed an anxiogenic-like effect in the LDB test.

DSGH (SEQ ID NO: 2) at a dose of 1 mg/kg has a potent anxiolytic-like effect in both LDB and NT tests.

DSGH (SEQ ID NO: 2) at a dose of 10 mg/kg failed to produce any behavioral changes in Danio rerio.

2.2.4. RAHE

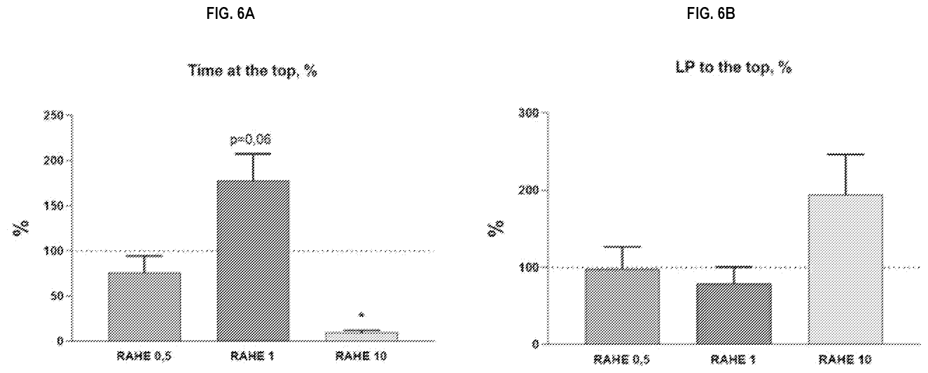

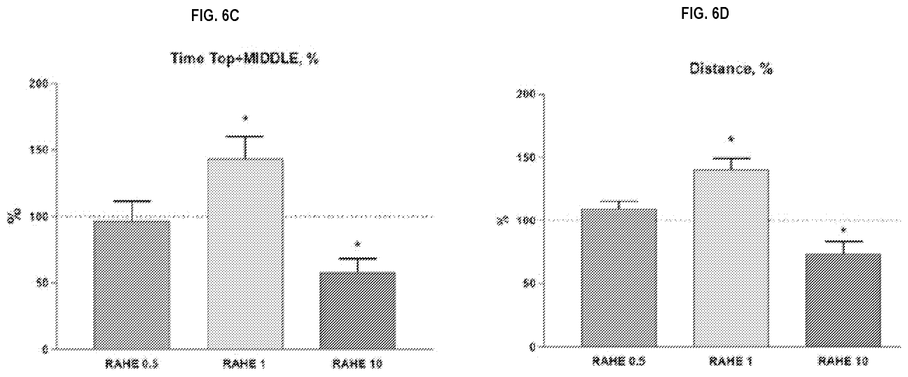

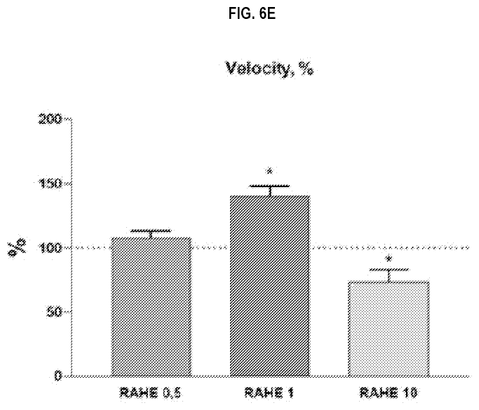

The administration of RAHE (SEQ ID NO: 3) at a dose of 0.5 mg/kg did not show any behavioral effects in NT and LDB tests (FIGS. 6A-6H).

After RAHE (SEQ ID NO: 3) injection at a dose of 1 mg/kg an increment of distance travelled was observed (FIG. 6D) and time spent in the upper ⅔ of the aquarium (FIG. 6C) and a trend towards increased time spent at the top of NT (p<0.10 Mann-Whitney U-test, FIG. 6A). The inventors propose a positive neurotropic effect of RAHE (SEQ ID NO: 3) peptide at a given dose, and the changes in behavioral parameters were similar to those, observed after ketamine treatment. At the same time, RAHE (SEQ ID NO: 3) treatment at a dose of 1 mg/kg did not show any behavioral effects in LDB test (data not shown).

The fish treated with RAHE (SEQ ID NO: 3) at a dose of 10 mg/kg had a prominent decrease of locomotor and exploratory activity in the NT test: the animals showed significant decrease of the time spent at the top of aquarium as well as at bot top+middle zones, decreased distance travelled and velocity (FIGS. 6A, 6C, 6D, 6E). These results may suggest a possible sedative or anxiogenic-like activity of RAHE (SEQ ID NO: 3) at a given dose.

Thus, the dose-dependent study of RAHE (SEQ ID NO: 3) effects in the NT and LDB tests revealed that:

RAHE (SEQ ID NO: 3) at a dose of 1 mg/kg has a prominent anxiolytic-like effects in NT but not LDB test.

RAHE (SEQ ID NO: 3) at a dose of 0.5 mg/kg did not affect the behavior of Danio rerio.

RAHE (SEQ ID NO: 3) at a dose of 10 mg/kg produced sedation or anxiety in fish.

2.2.5. KEDV

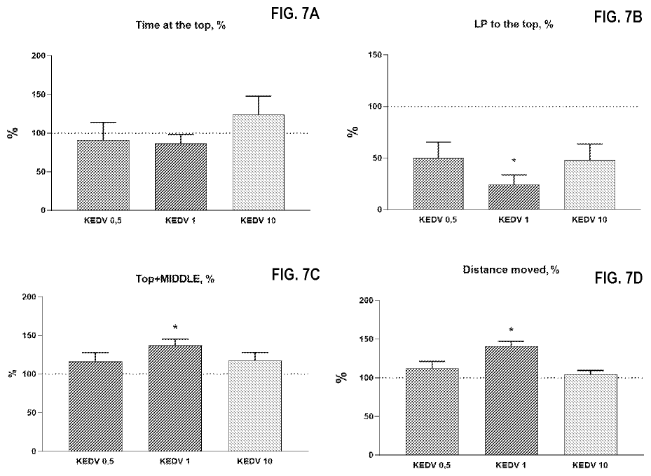

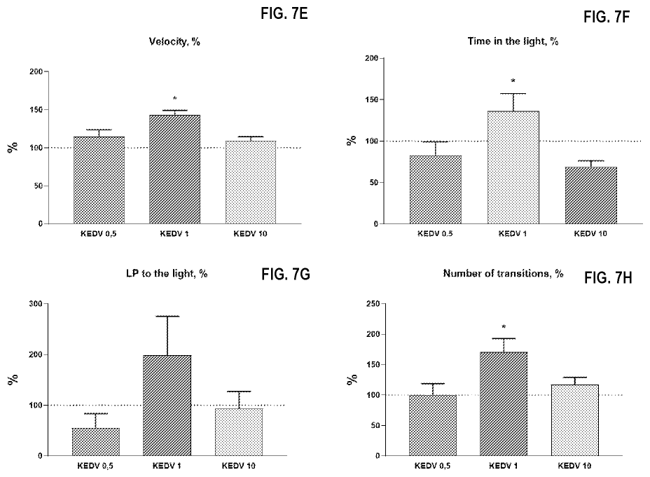

The KEDV (SEQ ID NO: 4) administration at a dose of 0.5 mg/kg did not change the behavior of fish neither in NT nor in LDB test (FIGS. 7A-7H).

The fish treated with KEDV (SEQ ID NO: 4) at a dose of 1 mg/kg showed an increased locomotor activity (FIG. 7D), increased time spent in the upper ⅔ of the aquarium (FIG. 7C) and decreased latency to the top (FIG. 7B). These positive neurotropic effects were similar to those observed after ketamine treatment and suggest decreased anxiety and increased exploratory activity in animals treated with KEDV (SEQ ID NO: 4) at a dose of 1 mg/kg. The injection of the peptide at a given dose also resulted in significant increase of entries to the light (FIG. 7H) and increased time spent in the light compartment (FIG. 7F) of LDB. The results obtained in NT and LDB tests suggest a potential anxiolytic-like effects of the peptide at a dose of 1 mg/kg.

The injection of KEDV (SEQ ID NO: 4) at a dose of 10 mg/kg did not alter the behavior of fish in an NT test (FIGS. 7A-7E), but, in an LDB test, it decreased time spent in the light compartment of the apparatus (FIG. 7F), which suggest anxiogenic-like effect of the given dose of peptide.

The results propose dose-dependent effects after KEDV (SEQ ID NO: 4) administration:

KEDV (SEQ ID NO: 4) at a dose of 0.5 mg/kg failed to produce any behavioral changes in Danio rerio.

KEDV (SEQ ID NO: 4) at a dose of 1 mg/kg has a potent anxiolytic-like effects in both LDB and NT tests.

KEDV (SEQ ID NO: 4) at a dose of 10 mg/kg has an anxiogenic-like effect in the LDB test.

The effects of AYFE (SEQ ID NO: 10) tetrapeptide on Danio rerio behavior.

2.2.6. KEDV

The AYFE (SEQ ID NO: 10) tetrapeptide was chosen as a negative control according to low scoring in the LOGO analysis.

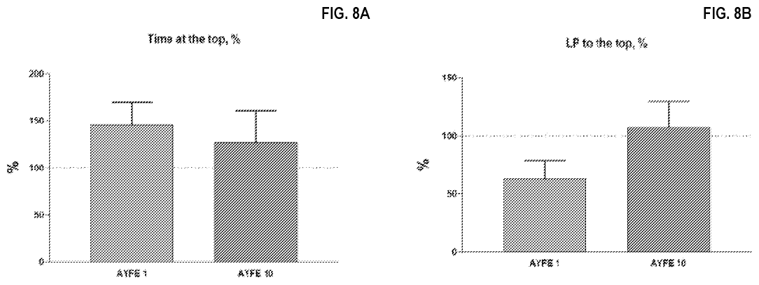

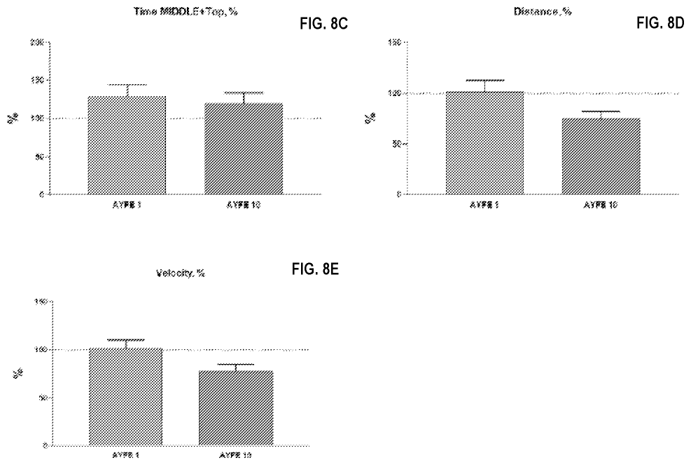

The AYFE (SEQ ID NO: 10) administration at a dose of 1 mg/kg did not alter the behavior of animals neither in NT (FIGS. 8A-8E) nor in LDB test (data not shown). The treatment with AYFE (SEQ ID NO: 10) at a dose of 10 mg/kg led to the attenuation of locomotor activity of fish in the NT test (FIG. 8D). The other parameters and the behavior of animals in the LDB test did not differ from control values. The decreased locomotion may suggest a sedative effect of the AYFE (SEQ ID NO: 10) peptide at a given dose.

The study of AYFE (SEQ ID NO: 10) peptide revealed that:

AYFE (SEQ ID NO: 10) at a dose of 1 mg/kg did not show behavioral effects in Danio rerio;

AYFE (SEQ ID NO: 10) at a dose of 10 mg/kg produced sedation in Danio rerio.

2.3. Conclusions

In the current study, the neurotropic effects of the peptides in Danio rerio were assessed. The peptides AGAS (SEQ ID NO: 1), DSGH (SEQ ID NO: 2), RAHE (SEQ ID NO: 3), and KEDV (SEQ ID NO: 4) were chosen according to molecular docking studies as potential mGRM5 receptor negative allosteric modulators.

The analysis of behavior of fish after treatment with such antidepressants as fluvoxamine (previous screening studies of GABA-A potent peptide agonists) and ketamine (the current study) has revealed that these substances primarily affect the behavior of fish in an test, by decreasing the time spent at the bottom of the aquarium. At the same time, the behavior of animals in an LDB test has changed to a lesser extent, and there was only a trend towards significant differences in such parameters as time spent in the light and the latency to visit the light compartment.

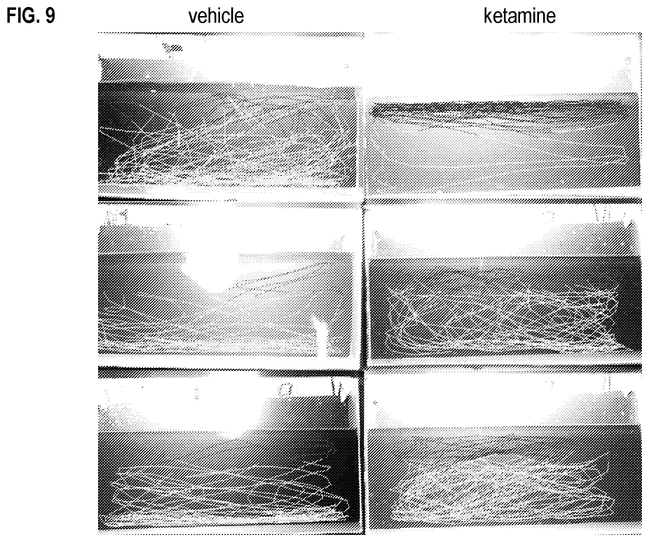

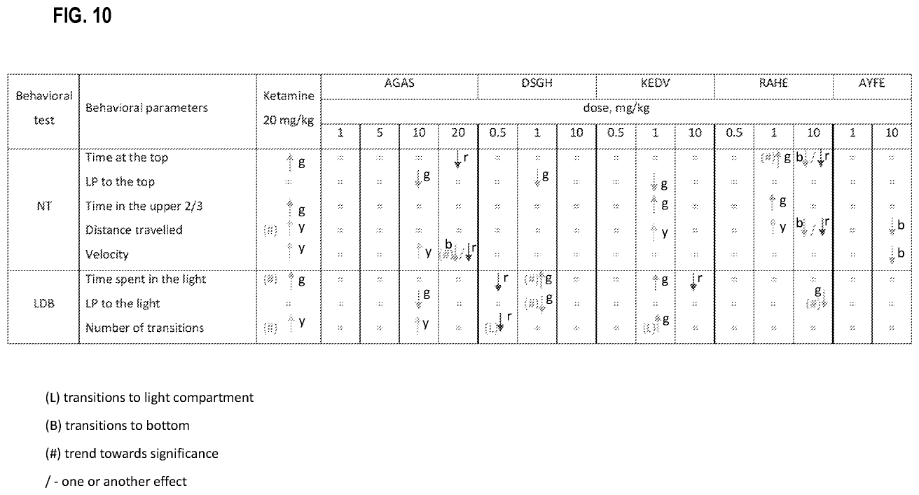

When comparing the results of tested tetrapeptides administration with reference drug ketamine, it was revealed that certain doses of some peptides have a similar effect in the NT test (FIG. 10). The treatment with KEDV (SEQ ID NO: 4) (1 mg/kg) and RAHE (SEQ ID NO: 3) (1 mg/kg) peptides resulted in significant increase of the time spent in the middle and at the top of aquarium (upper ⅔ of aquarium)—this effect was also observed after ketamine administration. There was also noted the similarity of the movement trajectories between the ketamine- and KEDV-treated animals: the fish from both groups preferred to swim at the top of aquarium (see FIG. 9). This result may indicate that the test substances may reduce anxiety-like behavior in fish.

Ketamine treatment also resulted in trend towards increased locomotor activity in NT test. It was previously shown that acute ketamine exposure produces hyperactivity in Danio rerio (Zakhary et al. (2011) A behavioral and molecular analysis of ketamine in zebrafish. Synapse. 65(2): 160-167). At the same time, KEDV (SEQ ID NO: 4) (1 mg/kg) and RAHE (SEQ ID NO: 3) (1 mg/kg) administration resulted in significant increase of distance travelled in fish.

The AGAS (SEQ ID NO: 1) (10 mg/kg) and DSGH (SEQ ID NO: 2) (1 mg/kg) and KEDV (SEQ ID NO: 4) (1 mg/kg) treatment resulted in increased latency to the top of the NT (FIG. 10). An increased latency to enter the top of aquarium in fish indicates a faster adaptation of animals to the novel conditions and thus this may indicate a decreased anxiety-like behavior of Danio rerio after treatment with peptides. Ketamine administration did not alter this parameter.

The most prominent effects in the LDB test were observed after KEDV (SEQ ID NO: 4) (1 mg/kg) and AGAS (SEQ ID NO: 1) (10 mg/kg) treatment: the peptides administration increased the time spent in the light or decreased the latency to visit the light compartment together with increase of the number of transitions between compartments. These results indicate an increased exploratory activity of Danio rerio and decreased anxiety after treatment with peptides. Ketamine and DSGH (SEQ ID NO: 2) at a dose of 1 mg/kg showed only trend towards reduction of anxiety-like behaviors in the LDB test.

The inventors conclude that the observed neurotropic effects caused by effective doses of the potential mGluR₅ negative allosteric peptide modulators are comparable to those effects of ketamine. A comparison of the track pattern after ketamine and KEDV (SEQ ID NO: 4) (1 mg/kg) treatment also confirms the similarity of the behavioral effects of the studied substances, and the further animal studies are required for better understanding of possible implication of the tested peptides.

### Example 3: Determination of Neurotropic Activity of GRM5 Receptor Peptide Modulators in Mice

The objective of this experiment was to identify the potential neurotropic effect of peptides with potent negative allosteric modulation of the mGluR-5 receptor on the behavior of BALB/C mice after acute intraperitoneal injections. The effects of administering of peptides on behavior of BALB/C mice was compared to the effects of administering Fluvoxamine (FA) to BALB/C mice. The effects of both peptides and FA on behavior of mice were assessed using the Open Field, the Elevated Plus Maze test, the Porsolt Forced Swim test (two-day modification). These behavioral paradigms are useful tools for evaluating neurotropic properties of novel drugs.

3.1. Materials and Methods

3.1.1. Animal Model

Eighty male BALB/C mice were used as subjects in this example. Body weight of each specimen at the beginning of the experiment was between about 18 grams and about 20 grams. All animals were free from species-specific pathogens (specific pathogen free (SPF) status according to the Federation of European Laboratory Animal Science Associations (FELASA) list, 2014). The animals were kept in conditions of free access to water and food. The room was air-conditioned (exchange rate not less than 15 r/h) with a 12 h:12 h light-dark cycle (lights on at 09:00 am), air temperature 20-24°±2° C. (possible fluctuations of the limits no more than 2° C. per day), 30-70% humidity. For the study, the mice were separated into six different groups and the tested substances were administered to the groups as shown in Table 1. Drugs were daily prepared in fresh saline, according to the dosage. For intranasal administration (i.n.), a volume of 20 μl was used, for intraperitoneal (i.p.)—200 μl.

TABLE 1
Experimental groups.

| Group name | Group | | Behavioral |
|---|---|---|---|
| and dose | size | Test Substance | tests |
| 1. Intact | 12 | Intraperitoneal injections | Elevated Plus |
| Control | | of solvent according to the | Maze test, Open |
| | | experimental group study design | Field test, Porsolt |
| 2. AGAS (SEQ | 11 | Intraperitoneal administration | Forced Swim test |
| ID NO: 1), 10 | | of AGAS at a dose of 10 mg/kg in | (two-day |
| mg/kg | | 30 minutes before each test | modification) |
| 3. DSGH | 11 | Intraperitoneal administration | |
| (SEQ ID NO: | | of DSGH at a dose of 1 mg/kg in | |
| 2), 1 mg/kg | | 30 minutes before each test | |
| 4. RAHE (SEQ | 11 | Intraperitoneal administration | |
| ID NO: 3), 1 | | of RAHE at a dose of 1 mg/kg in | |
| mg/kg | | 30 minutes before each test | |
| 5. KEDV (SEQ | 11 | Intraperitoneal administration | |
| ID NO: 4), 1 | | of KEDV at a dose of 1 mg/kg in | |
| mg/kg | | 30 minutes before each test | |
| 6. AYFE (SEQ | 12 | Intraperitoneal administration | |
| ID NO: 10), 1 | | of AYFE at a dose of 1 mg/kg in | |
| mg/kg | | 30 minutes before each test | |
| 7. Comparison | 12 | Intraperitoneal administration | Porsolt Forced |
| drug FA | | of comparison drug Fluvoxamine | Swim test (two- |
| | | at a dose of 20 mg/kg in 30 | day modification) |
| | | minutes before each test. | |

After an adaptation period, the test substance was intraperitoneally injected into mice. Behavioral parameters were measured in 30 minutes after injection. No more than one test was performed per day. The tests were administered as follows: day 1—the Open field test, day 8—the Elevated plus maze test, days 15-16—the Porsolt Forced Swim (two-day modification) test.

3.1.2. Statistical Analysis

Statistical data analysis was performed using one-way analysis of variance (ANOVA) followed by Fisher's LSD test for normally distributed data.

3.1.3. The Open Field Test

The test is an arena with a diameter of 63 cm, illuminated by bright light (500 Lx). Recorded parameters: total distance traveled (in cm), time spent moving (at a speed of more than five cm/s), time spent immobile (at a speed of less than 0.2 cm/s), average and maximum velocity, the number of episodes of motor activity and freezing. The same set of parameters, as well as the latent period and the time spent are recorded for the central sector. Defecation, rears (setting the animal on its hind legs) are evaluated visually. The test is designed to assess the level of motor and exploratory activity.

3.1.4. The Elevated Plus Maze Test

The test consists of two closed and two open arms located opposite each other (arm length 30 cm). The height of the sides of the closed arms is 15 cm. The entire installation is raised 70 cm above the floor. Open arms have a bright uniform illumination of 400 lux, closed −30-40 lux. The animal is placed in the center of the maze, with its head facing an open arm. Within five minutes, the Noldus EthoVision XT program (for behavior tracking) automatically records the following behavior parameters: total distance traveled (in cm), time spent moving (at a speed of more than five cm/s), time spent immobile (at a speed of less than 0.2 cm/s), average and maximum velocity, the number of episodes of mobility, and freezing. The same set of parameters, as well as the latent period and the time spent, are recorded for the central sector, the open and closed arms separately.

3.1.5. The Porsolt Forced Swim Test (Two-Day Modification)

As part of this modification, two tests were carried out within two days. The installation is a transparent cylinder, 30 cm high, 10 cm in diameter and filled with water (water temperature 21° C.-23° C.) to a mark at a height of 25 cm. On the first day, each animal was dropped into the cylinder for 10 minutes. Behavioral parameters were not recorded. The animals are dropped into the cylinder for ten minutes again on the second day. The duration of active (vigorous movements with all paws) and passive (weak strokes with hind legs) swimming, as well as immobility (immobilization) (Porsolt et al. (1977). Behavioral despair in mice: a primary screening test for antidepressants. Arch Int Pharmacodyn Ther, 229(2), 327-336) are recorded. After each test the mice are placed in a heated cage until dry. The behavior indicators in this test are processed using the Real Timer Program developed by NPK “OpenScience” LLC, Russia. The sequence of events and their duration were recorded as well as basic statistical data processing was conducted.

3.2. Evaluation of Neurotropic Effects of Test Substances

3.2.1. Evaluation of the Effects of Test Substances on Mice Behavior in the Open Field Test

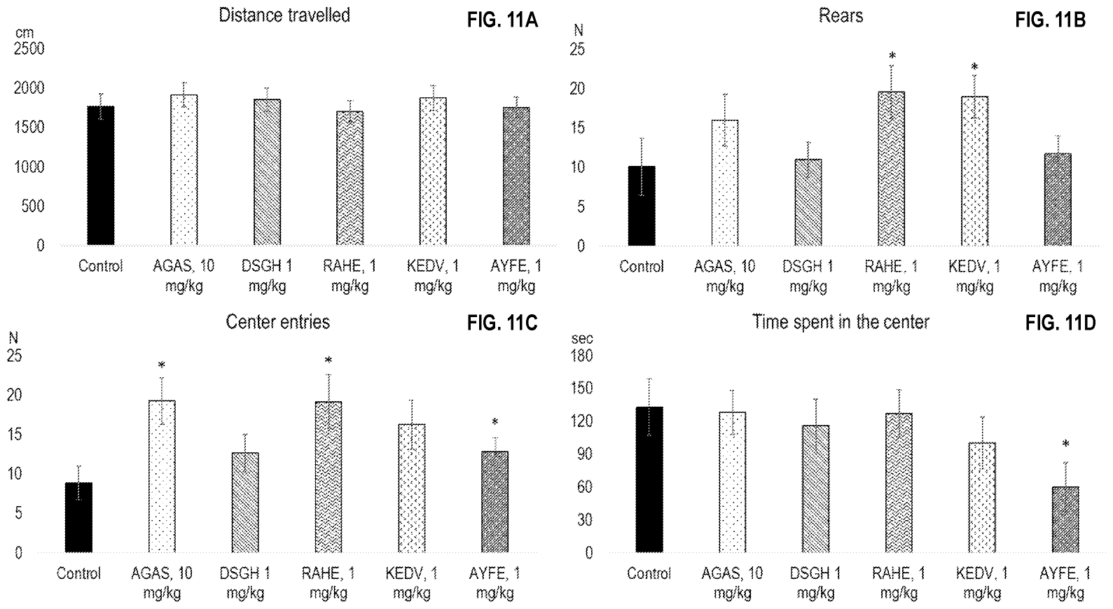

To assess the effect of drugs on motor (total distance traveled) and exploratory activity (number of rears, number of center entries, as well as time spent in the center), animals were tested in the Open Field test. According to the parameter of the total distance traveled, no differences from the control were observed in any of the studied groups (FIG. 11A).

RAHE (SEQ ID NO: 3) and KEDV (SEQ ID NO: 4) administration significantly increased the number of rears (19.6±3.4; 19±2.7) (FIG. 11B), which suggests the anxiolytic-like action of these compounds due to a shift in the behavioral reaction towards exploration. An increase in the number of center entries can also indicate the presence of anxiolytic-like action and was shown for the peptides AGAS (SEQ ID NO: 1) and RAHE (SEQ ID NO: 3) (19.3±3 and 19.2±3.4, respectively) (FIG. 11C). However, there were no differences in the time spent in the center between the groups (FIG. 11D). The time spent in the center in animals treated with AYFE (SEQ ID NO: 10) peptide (59.96±22.4) were significantly lower in comparison with control group (132.9±25.9) (FIG. 11D). This indicates increased thigmotaxis in this group, revealing the anxiogenic-like effect of the peptide.

3.2.2. Evaluation of the Effects of Test Substances on Mice Behavior in the Elevated Plus Maze Test

According to the standard protocol, two groups of behavioral parameters are distinguished in the test. The first group reflects the motor activity of animals: freezing time (i.e., time without movement, s), total distance traveled (cm), and the number of rears on the closed arms. An increment of these parameters may indicate a decrease in the passive defensive behavior with a shift towards exploration in animals. This can be interpreted as a manifestation of the anxiolytic-like effect of the drug.

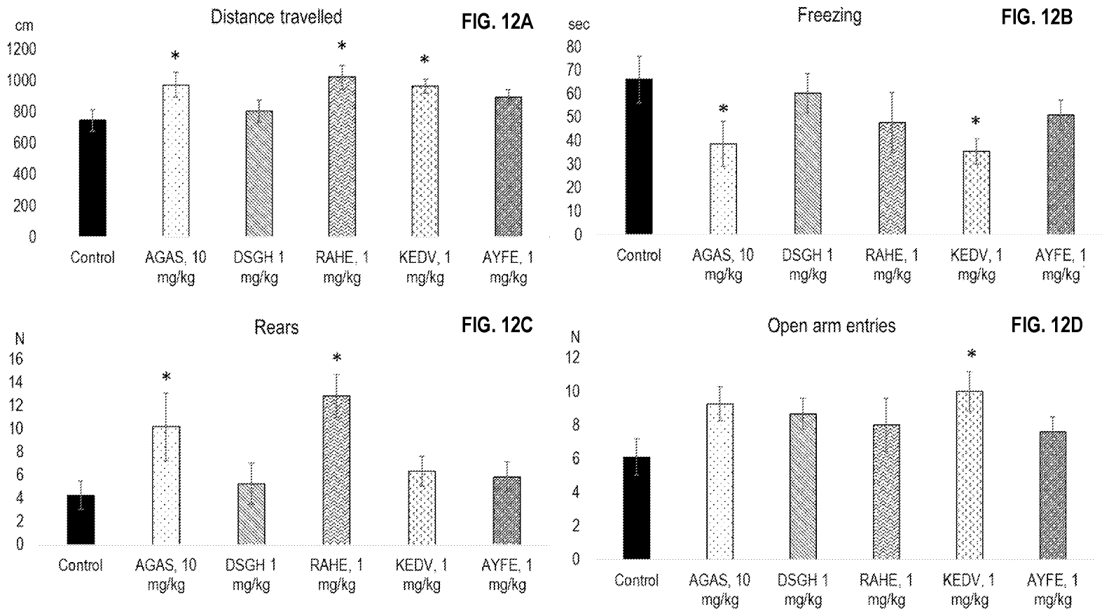

An increase in the distance travelled was observed in animals treated with AGAS (SEQ ID NO: 1) (973±78.7), RAHE (SEQ ID NO: 3) (1021±76.6) and KEDV (SEQ ID NO: 4) (963.9±43.3) peptides (FIG. 12A). The freezing time was decreased in the AGAS (SEQ ID NO: 1) (38.7±9.6) and KEDV (SEQ ID NO: 4) (35.5±5.5) groups in comparison with control group of animals (FIG. 12B). For the number of rears on the closed arms (FIG. 12C), statistically significant differences from the control group (4.3±1.2) were obtained for the peptides AGAS (SEQ ID NO: 1) (10.2±2.9) and RAHE (SEQ ID NO: 3) (12.8±1.9) which also indicates a shift in behavior from a defensive to exploratory motivation.

The second group of parameters include: the time spent on the open arms, the number of open arms entries. The time spent on the open arms of the maze didn't differ between groups.

The animals which received KEDV (SEQ ID NO: 4) peptide showed the increase in the number of open arm entries in comparison with animals from control group (FIG. 12D) which may reflect the decreased anxiety-like behavior in experimental group.

3.2.3. Evaluation of the Effects of Test Substances on Mice Behavior in the Porsolt Forced Swim Test.

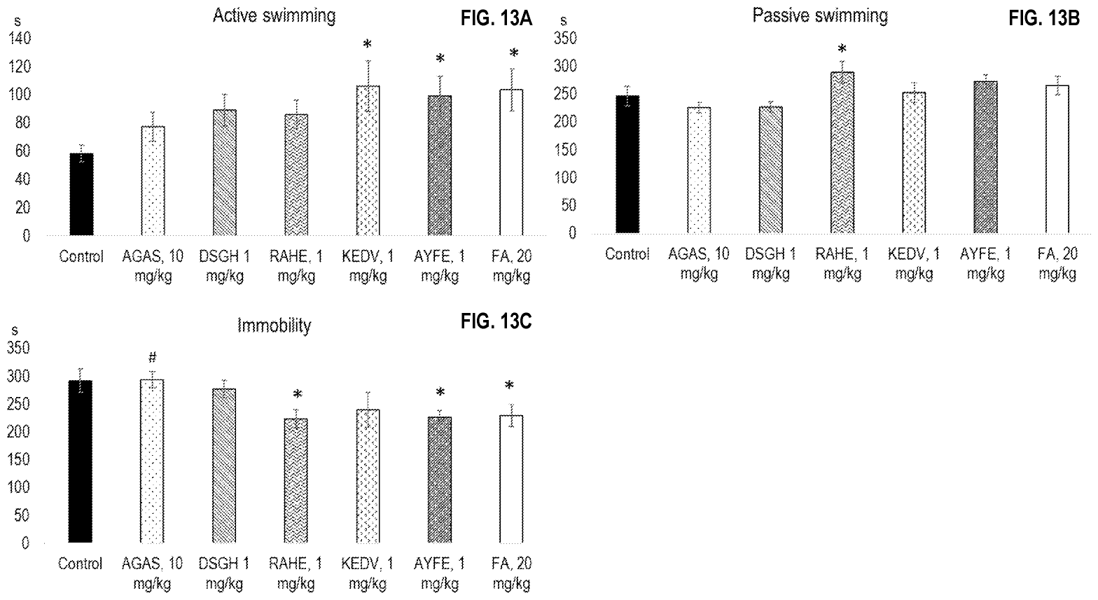

A two-day modification of the test allows the animal to adapt to the experimental setup during the first day, which leads to more specific changes in behavior that can be interpreted as a “behavioral despair”. The FA treatment reduced the time spent immobile (229±19.8 s vs. 292.1±21.2 s in the control group) and increased the time of active swimming (103.5±15.1 s vs. 58.3±6.1 sin the control group) (FIGS. 13A, 13C).

Among the tested peptides the most pronounced antidepressant-like properties were shown for RAHE (SEQ ID NO: 3) and KEDV (SEQ ID NO: 4) groups. The administration of the RAHE (SEQ ID NO: 3) peptide at a dose of 1 mg/kg resulted in decreased time spent immobile (223.6±16.1 s), as well as increased passive swimming (floating using two or four limbs) relative to control values (289±19.6 s and 246.4±17.7 s, respectively) (FIGS. 13B, 13C). The treatment with the KEDV (SEQ ID NO: 4) peptide resulted in increased time spent active swimming (FIG. 13A). The time spent immobile was decreased in the group of animals which received AYFE (SEQ ID NO: 10) and the time spent active swimming was increased in comparison with control group (FIGS. 13A, 13C). These results indicate antidepressant-like properties of the peptide.

3.3. Discussion of the Results

The peptide AGAS (SEQ ID NO: 1) at a dose of 10 mg/kg did not have a pronounced antidepressant-like effect. However, the AGAS (SEQ ID NO: 1) administration led to an increase in exploratory and motor activity, which may propose the anxiolytic-like effects of this peptide. The administration of DSGH (SEQ ID NO: 2) at a dose of 1 mg/kg did not cause any changes of the behavioral parameters. The treatment with RAHE (SEQ ID NO: 3) and KEDV (SEQ ID NO: 4) peptides at a dose of 1 mg/kg resulted in increased motor and exploratory activity in OF and EPM tests and decreased behavioral despair in FST. An increment of passive swimming in mice after RAHE (SEQ ID NO: 3) administration may propose a shift towards energy saving passive-coping strategy on the second day of testing. According to the results of the study, these two peptides produce anxiolytic-like and antidepressant-like effects and are the most promising for further studies on neurotropic activity of the drugs.

The AYFE (SEQ ID NO: 10) peptide at a dose of 1 mg/kg increased thigmotaxis in the OF test, though it didn't affect the behavior of mice in EPM. In the FST the administration of the substance produced an antidepressant-like effect. The additional studies required to estimate the behavioral effects of this peptide.

3.4. Conclusions

The treatment with AGAS (SEQ ID NO: 1) peptide (SEQ ID NO: 1) at a dose of 10 mg/kg resulted in moderate increase of exploratory and motor activity in OF and EPM but did not affect the behavior of animals in FST.

The treatment with DSGH (SEQ ID NO: 2) peptide at a dose of 1 mg/kg did not affect the behavior of mice.

The treatment with RAHE (SEQ ID NO: 3) and KEDV (SEQ ID NO: 4) peptides at a dose of 1 mg/kg 30 minutes prior to behavioral tests led to a pronounced antidepressant-like effect similar to those observed after FA treatment, and anxiolytic-like effects.

The peptide AYFE (SEQ ID NO: 10) at a dose of 1 mg/kg showed an antidepressant-like effect and at the same time increased thigmotaxis.

### Example 4: Neurotropic Activity of Peptide Drugs when Administered Intranasally at Various Doses

4.1. The Objective of the Study:

Investigation the potential neurotropic effects of several novel peptides at various doses and to evaluate the effects of these compounds on the motor activity of laboratory Sprague-Dawley rats.

4.2. Materials and Methods

4.2.1. Animal Model

The experiment was carried out on 70 male Sprague-Dawley rats. The body weight by the beginning of the experiment was 150-200 g. All animals were free from species-specific pathogens (SPF status according to the FELASA list, 2014). The animals were kept in conditions of free access to water and food. The room was air-conditioned (exchange rate not less than 15 r/h) with a 12 h:12 h light-dark cycle (lights on at 09:00 am), air temperature 20-24°±2° C. (possible fluctuations of the limits no more than 2° C. per day), 30-70% humidity. For the study, the rats were separated into eight different groups and the tested substances were administered to the groups as shown in Table 2. Drugs were daily prepared in fresh saline, according to the dosage. For intranasal administration (i.n.), a volume of 20 μl was used, for intraperitoneal (i.p.)—200 μl.

At the end of the adaptation period the animals were treated with studied drugs and after 30 minutes behavioral testing has been carried out (the comparison drug was administered i.p). The list and order of planned tests were: Open Field (OF)—on the 1^{st} day, Novelty Suppressed Feeding (NSF)—on the 2^{nd} day, Novel Object Recognition (NOR)—day 3 and 4, day 8—Elevated Plus Maze (EPM).

TABLE 2
Experimental groups.

| | Group | | Behavioral |
|---|---|---|---|
| Group Name | size | Test Substance | tests |
| 1. Control | 10 | Intranasal (i.n.) administration | OF, NSF, NOR, EPM |
| | | of saline | |
| 2. RAHE, 0.1 mg/kg | 10 | l.n. administration of | |
| | | RAHE 0.1 mg/kg | |
| 3. RAHE, 0.3 mg/kg | 10 | l.n. administration of | |
| | | RAHE 0.3 mg/kg | |
| 4. RAHE, 1 mg/kg | 10 | l.n. administration of | |
| | | RAHE 1 mg/kg | |
| 5. KEDV, 0.1 mg/kg | 10 | l.n. administration of | |
| | | KEDV 0.1 mg/kg | |
| 6. KEDV, 0.3 mg/kg | 10 | l.n. administration of | |
| | | KEDV 0.3 mg/kg | |
| 7. KEDV, 1 mg/kg | 10 | l.n. administration of | |
| | | KEDV 1 mg/kg | |
| 8. Fluoxetine 5 mg/kg | 12 | l.p. administration of | FST |
| | | fluoxetine 20 mg/kg | |

4.2.2. Statistical Analysis

Statistical data analysis was performed using one-way analysis of variance (ANOVA) followed by Fisher's LSD test for normally distributed samples.

4.2.3. The Open Field Test (OF)

The test is an arena with a diameter of 97 cm, illuminated by bright light (500 Lx). Recorded parameters: total distance traveled (in cm), time spent moving (at a speed of more than five cm/s), time spent immobile (at a speed of less than 0.2 cm/s), average and maximum velocity, the number of episodes of motor activity and freezing. The same set of parameters, as well as the latent period and the time spent are recorded for the central sector. Defecation, rears (setting the animal on its hind legs) are evaluated visually. The test is designed to assess the level of motor and exploratory activity.

4.2.4. Novelty Suppressed Feeding Test (NSF)

Before testing, animals were deprived of food for 8 hours. On the next day, a Petri dish with 2 food pellets were placed in the center of the OF setup. Testing time was 5 minutes. Video processing was carried out using the EthoVision XT14 (Noldus) video tracking system. Recorded parameters were the number of rears, grooming, defecation, average speed (cm/s), total distance traveled (cm), time spent immobile (s), the number of center entries and time spent at the center of the testing area (the animal's nose should be within a one-centimeter proximity of the food pellet) and the total time spent in the center (s).

This test investigates food motivation under novel conditions. This test is standard for the study of anxiolytic-like properties of the drugs. The greater the number of approaches to the food pellet and the time spent in the center, the higher the anxiolytic potential of the studied compounds (Ran et al. (2018). YL-0919, a dual 5-HT 1A partial agonist and SSRI, produces antidepressant-and anxiolytic-like effects in rats subjected to chronic unpredictable stress. APS. 39(1): 12).

4.2.5. Novel Object Recognition (NOR)

The test was performed in 3 days. The 1^{st} day is a standard OF test. On the 2^{d} day, two identical objects (green plastic pyramids) were placed at a distance of 30 cm from the walls of the apparatus, opposite to each other. For the 3^{d} day, one of the green pyramids was replaced by a blue pyramid. Each day the behavior of the animals was observed for 5 minutes. Video processing is carried out using the EthoVision XT14 (Noldus) video tracking system. Recorded parameters were the number of rears, grooming, defecations, average speed (cm/s), total distance traveled (cm), immobility time (s), the number of approaches and time spent near the objects (the animal's nose should be within a one-centimeter proximity of the object) and the total time spent in the center (s).

Normally, animals tend to explore new objects and the number of approaches, and sniffing of the object increases. When memory is impaired, animals do not perceive the object on the 2^{d} day as new, which is reflected in a decrease of exploratory behavior (the number of approaches and time spent with the new object) relative to the control group (Antunes et al. (2012). The novel object recognition memory: neurobiology, test procedure, and its modifications. Cogn. Process. 13(2): 93-110).

4.2.6. The Elevated Plus Maze Test (EPM)

The test consists of two closed and two open arms located opposite each other (arm length 50 cm). The height of the sides of the closed arms is 30 cm. The entire installation is raised 70 cm above the floor. Open arms have a bright uniform illumination of 400 lux, closed −30-40 lux. The animal was placed in the center of the maze, with its head facing an open arm. Within 5 minutes, the EthoVision XT14 (Noldus) program automatically records the following behavioral parameters: total distance traveled (in cm), time spent moving (at a speed of more than five cm/s), time spent immobile (at a speed of less than 0.2 cm/s), average and maximum speed, the number of episodes of mobility, and freezing. The same set of parameters, as well as the latent period and the time spent, are recorded for the central sector, the open and closed arms separately. “Anxiety Index” index was also calculated by the following formula: Al =100*(1−(time on open arms/total test time+open arms entries/total number of entries)/2).

4.2.7. Modified Porsolt Forced Swim Test (FST)

As part of this modification, two tests are carried out within two days. The installation is a transparent cylinder, 30 cm high, 10 cm in diameter and filled with water (water temperature 21° C.-23° C.) to a mark at a height of 25 cm. On the first day, each animal is placed in the apparatus for ten minutes. Behavioral parameters are not recorded. The animals are placed in the cylinder for 10 minutes again on the second day. The duration of active (vigorous movements with all paws) and passive (weak strokes with hind legs) swimming, as well as immobility (immobilization) (Porsolt et al. (1977) Behavioral despair in mice: a primary screening test for antidepressants. Arch Int Pharmacodyn Ther. 229(2):327-36) are recorded.

After each test, the rats are placed in a heated cage until dry. The behavioral indicators in this test are processed using the Real Timer Program developed by Open Science. The sequence of events, their duration, as well as conduct basic statistical data processing are recorded.

4.3. Results

4.3.1. Open Field Test

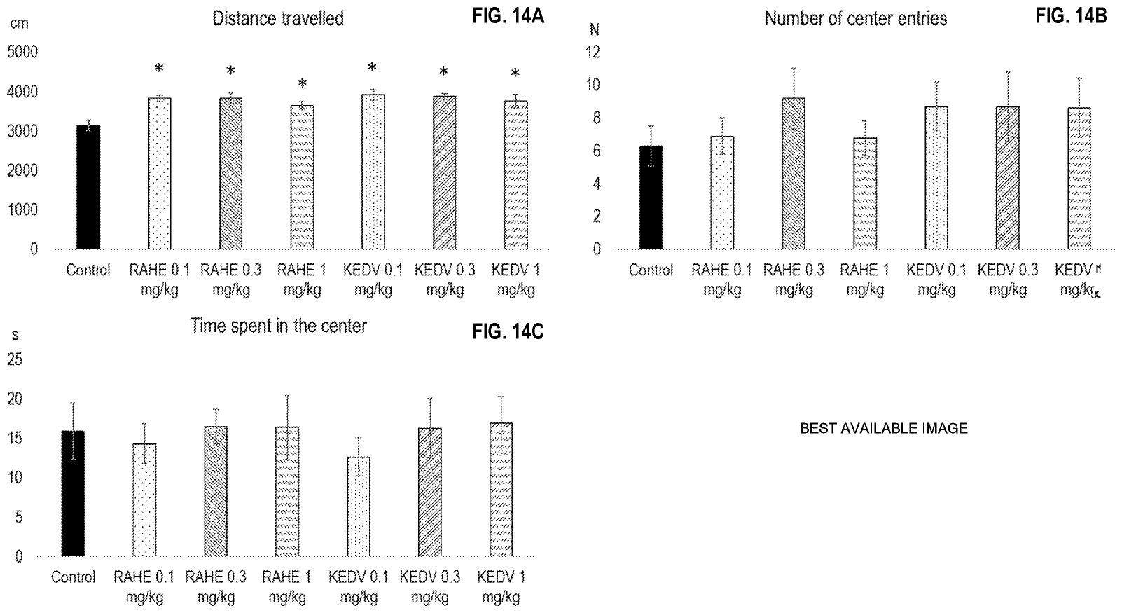

The OF test was used to assess the motor and exploratory activity of animals after administration of the studied substances. The administration of both RAHE (SEQ ID NO: 3) and KEDV (SEQ ID NO: 4) peptides at doses of 0.1, 0.3 and 1 mg/kg led to an increase of distance traveled by more than 15% (RAHE 0.1 mg/kg (3835±80.6 cm), RAHE 0.3 mg/kg (3838±137.2 cm), RAHE 1 mg/kg (3651±108.7 cm), KEDV (SEQ ID NO: 4) 0.1 mg/kg (3920±134.8 cm), KEDV (SEQ ID NO: 4) 0.3 mg/kg (3879±73.7 cm), KEDV (SEQ ID NO: 4) 1 mg/kg (3769±164.7 cm) (FIG. 14A).

However, there were no effects of peptides administration on the number of center entries (FIG. 14B) and the time spent in the center of the maze (FIG. 14C).

4.3.2. Novelty Suppressed Feeding Test

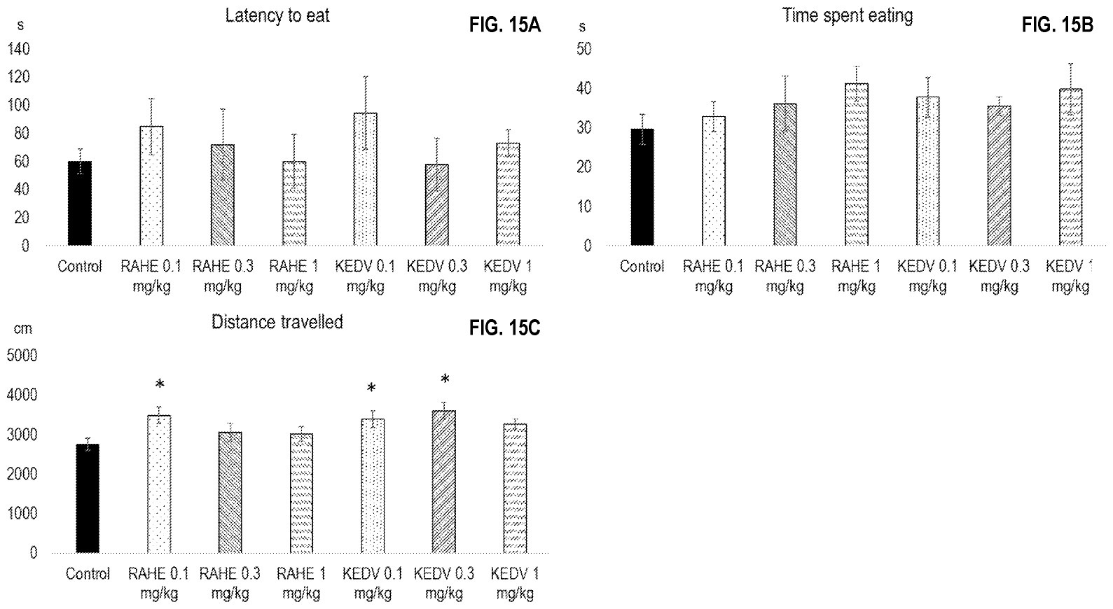

There were no effects of the tested peptides on the latency to eat (FIG. 15A) and the time spent eating (FIG. 15B) in all experimental groups, which suggest the absence of anxiolytic-like effects of the peptides in this test.

However, the similar effect on locomotor activity observed earlier in the OF test was found in NSF. In some peptide-treated groups of animals there were a significant increases in the total distance traveled (RAHE 0.1 mg/kg (3490±212.3 cm), KEDV (SEQ ID NO: 4) 0.1 mg/kg (3392±211.3 cm) and KEDV (SEQ ID NO: 4) 0.3 mg/kg (3608±208.6 cm)) in comparison with the control group (2758±149.2 cm) (FIG. 15C).

4.3.3. Novel Object Recognition

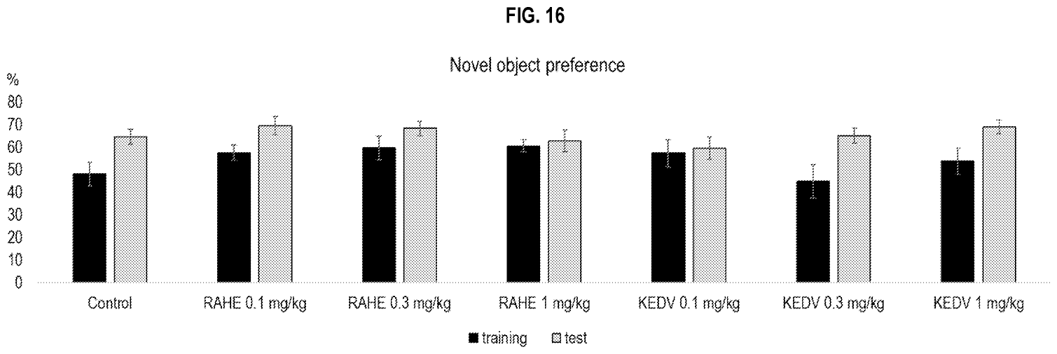

This test evaluates cognitive functions of laboratory animals after administration of the test compounds. Because rodents have an innate preference for novelty, a rodent that remembers the familiar object will spend more time exploring the novel object. Normally, the value of this parameter should be statistically significant and differ from the values obtained on the first day of the experiment. This is necessary to assess the validity of the test. According to the presented data (FIG. 16), most animals spent more time at the new object on the second day. However, there were no effect observed for the factor “experimental group”, which suggests no differences between the treatments. This result suggests normal cognitive functions in all experimental groups.

4.3.4. Elevated Plus Maze

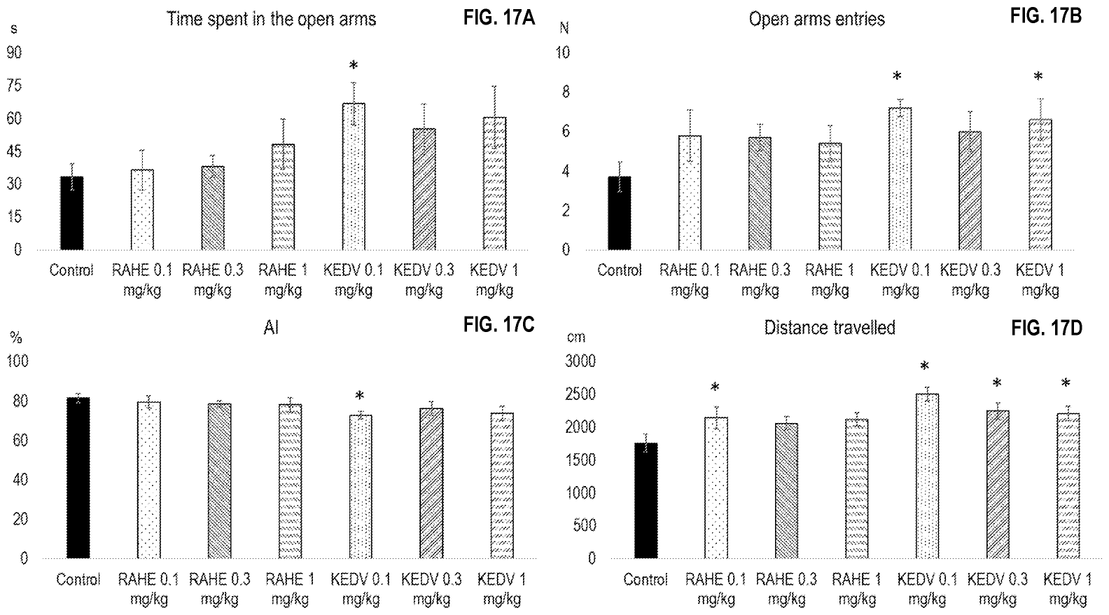

The treatment with KEDV (SEQ ID NO: 4) 0.1 mg/kg resulted in the increased time spent in the open arms (66.8±9.6 s) in comparison with the control group (33.5±6.02 s) (FIG. 17A). Open arms entries for this group was also higher than control values (7.2±0.44 and 3.7±0.76, respectively) and a similar increase was recorded for the KEDV (SEQ ID NO: 4) 1 mg/kg group (6.6±1.06 cm) (FIG. 17B). The anxiety index (AI) was significantly lower than the control values (81.3±2.32%) in the KEDV (SEQ ID NO: 4) 0.1 mg/kg group (72.8±1.96%) (FIG. 17C). This result may indicate the anxiolytic-like effect of this peptide.

Some doses of peptides enhanced locomotion in rats, which was expressed as significant increase in total distance travelled in RAHE 0.1 mg/kg (2146±170 cm), KEDV 0.1 mg/kg (2505±104.2 cm), KEDV 0.3 mg/kg (2247±127.8 cm) and KEDV 1 mg/kg (2209±111.2 cm) groups in comparison with control group (17.61±139 cm) (FIG. 17D).

4.3.5. Modified Porsolt Forced Swim Test

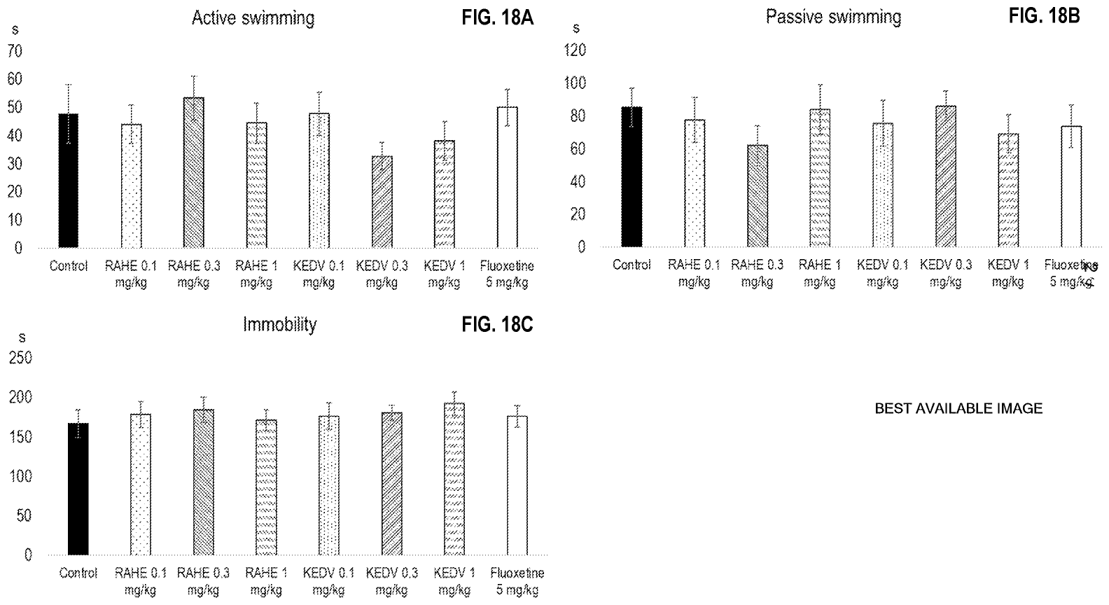

There were no changes of behavior observed in the FST between experimental groups (FIGS. 18A, 18B, and 18C), which suggest that none of the tested substances showed potent antidepressant-like activity.

4.4. Discussion

The enhanced motor activity induced by intranasal administration of peptides RAHE (SEQ ID NO: 3) and KEDV (SEQ ID NO: 4) was observed in almost every dose. The increase in the total distance travelled was significantly higher in peptide-treated groups in comparison with the control group in the OF, NSF and EPM tests. Thus, further studies are required, along with other conditions affecting motor and psychosomatic dysfunctions such as attention deficit disorder, Parkinson's disease, dyskinesia of various etiologies (e.g., tardive or levodopa-induced dyskinesia), etc.

Among the tested peptides at various doses, the potential moderate anxiolytic-like effect was observed only for KEDV (SEQ ID NO: 4) at a dose of 0.1 and 1 mg/kg. The peptide at a smallest dose increased time spent in the open arms of the EPM, and both doses of KEDV (SEQ ID NO: 4) were able to increase the number of open arm entries in this test. However, interpretation of the results is difficult since the observed changes can be a consequence of both anxiolytic-like properties of the studied substances (due to the shift towards exploratory behavior) and/or psychostimulant properties of the peptide drugs.

There was no anxiolytic-like effect observed in the NSF test after treatment with peptides. In the FST—the tests with predictive validity for the drugs with potential antidepressant-like activity,—there were no effects observed of administered peptides on studied parameters. Antidepressant-like properties were not observed neither in Fluoxetine nor in the peptide-treated groups. According to available literature data on fluoxetine, antidepressant-like activity can be clearly demonstrated in the models of stress, for example in the model of chronic stress in rats (Farhan et al. (2016) Anxiolytic profile of fluoxetine as monitored following repeated administration in animal rat model of chronic mild stress. SPJ, 24(5): 571-578). Thus, testing the peptide's activity on similar models of stressed animals may be more appropriate and recommended for further research.

4.5. Conclusions

Intranasal administration of peptides RAHE (SEQ ID NO: 3) and KEDV (SEQ ID NO: 4) at doses of 0.1, 0.3 and 1 mg/kg 30 minutes prior to behavioral testing significantly increased motor activity.

Intranasal administration of the peptide KEDV (SEQ ID NO: 4) 30 minutes prior to behavioral testing resulted in potential anxiolytic-like effect in the EPM at a dose of 0.1 and 1 mg/kg.

Intranasal administration of peptides RAHE (SEQ ID NO: 3) and KEDV (SEQ ID NO: 4) at doses of 0.1, 0.3 and 1 mg/kg 30 minutes prior to behavioral testing did not affect memory formation.

### Example 5: The Assessment of the Effect of the Studied Peptides on the Activity of Signaling Cascades Triggered Through the mGluR₅ Receptor, Assessed by the Efficiency of β-Arrestin2 Recruitment

5.1. The Objective of the Study:

Investigation the potential effects of studied peptides KEDV (SEQ ID NO: 4) and RAHE (SEQ ID NO: 3) as potential negative allosteric modulators of mGluR₅ using luciferase assay.

5.2. Principle of Method

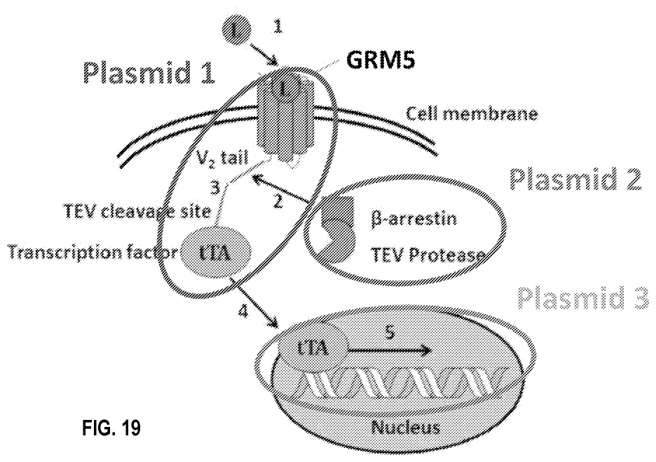

The methodology was previously described by Kroeze (Kroeze et al. (2015). PRESTO-Tango as an open-source resource for interrogation of the druggable human GPCRome. Nat. Struct. Mol. Biol. 22(5): 362) and the principle is illustrated in FIG. 19. Briefly, the cells of HEK293 cell line are transfected with 3 types of plasmids:

Plasmid 1: encodes fusion GRMS-tTA protein. The linker between GRMS and tTA is sensitive to TEV-protease (www.addgene.org/66390/).

Plasmid 2: encodes β-arrestin2/TEV-protease fusion protein (www.addgene.org/107245/).

Plasmid 3: luciferase tTA reporter (www.addgene.org/64127/).

Sufficient quantities of fusion proteins were produced during 24 hours after transfection. mGluR₅ (GRM5) activation by a selective agonist CHPG (SEQ ID NO: 18) (1) leads to recruiting of β-arrestin2/TEV-protease fusion protein (2), that cleaves the linker between GRM5 and tTA (3). Released tTA transcription factor (4) binds with its consensus sites in tTA luciferase reporter plasmid (5) that results in activation of luciferase expression. The activity of luciferase can be used to estimate the activity of mGluR₅, and application of negative allosteric modulators of mGluR₅ should lead to a dose-dependent decrease in luciferase activity in this system (FIG. 19).

5.3. Experimental Procedures.

Sufficient quantities of the plasmids were produced using standard applications before the experiment. HEK293 cells were co-transfected with three plasmids using polyethylenimine (PEI, 408727, Sigma-Aldrich, USA) 24 hours prior to agonists application. Transfected cells were treated with agonists/antagonists and incubated overnight (16 hours). Antagonists were introduced 10 minutes prior to agonists. Luciferase test was performed using Promega™ Luciferase Assay Systems Kit (PR-E1500, Promega, USA) according to the manufacturer's protocol.

5.3.1. Agonists/Antagonists:

CHPG—orthosteric selective mGlu5 receptor agonist (HB0033, HelloBio, USA). CHPG (SEQ ID NO: 18) was administered at a dose 1 mM according to the literature (Chen et al. (2012). Protective effects of mGluR₅ positive modulators against traumatic neuronal injury through PKC-dependent activation of MEK/ERK pathway. Neurochem. Res. 37(5): 983-990.; Loane et al. (2009). Activation of metabotropic glutamate receptor 5 modulates microglial reactivity and neurotoxicity by inhibiting NADPH oxidase. J. Biol. Chem. 284(23): 15629-15639).

SIB 1757—selective, noncompetitive antagonist of mGluR₅ (S9186, Sigma-Aldrich, USA). SIB 1757 was administered at a dose of 10 μM (according to the literature: Liu et al. (2014). Aldehyde dehydrogenase 1 defines and protects a nigrostriatal dopaminergic neuron subpopulation. J. Clin. Invest. 124(7): 3032-3046) and 100 μM (excessive dose).

Peptides RAHE (SEQ ID NO: 3) and KEDV (SEQ ID NO: 4)—potential negative allosteric modulators of mGluR₅. Peptide's dose of 0.2 μM was calculated from in vivo studies, in which these peptides demonstrated functional activity. Doses 2 μM and 20 μM are also physiologically relevant.

The results were calculated as the mean of 3 biological replicates.

5.4. Results

5.4.1. Part 1. Validation of Peptides' Efficiency at Low, Physiological Doses.

Experimental design:

Control. Non-transfected control, PEI-treated cells

Transfected control

Transfected cells+CHPG

Transfected cells+CHPG+SIB 1757, 10 μM

Transfected cells+CHPG+SIB 1757, 100 μM

Transfected cells+CHPG+RAHE (SEQ ID NO: 3), 0.2 μM

Transfected cells+CHPG+RAHE (SEQ ID NO: 3), 2 μM

Transfected cells+CHPG+RAHE (SEQ ID NO: 3), 20 μM

Transfected cells+CHPG+KEDV (SEQ ID NO: 4), 0.2 μM

Transfected cells+CHPG+KEDV (SEQ ID NO: 4), 2 μM

Transfected cells+CHPG+KEDV (SEQ ID NO: 4), 20 μM

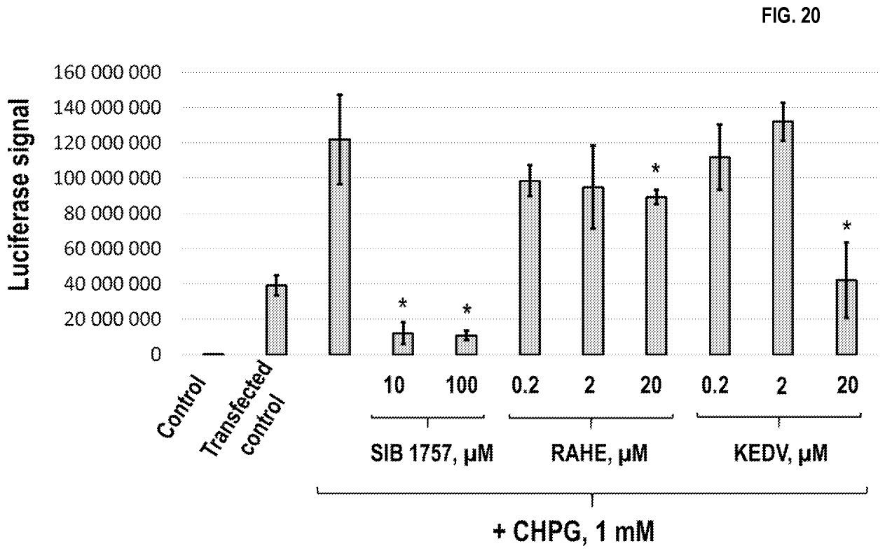

The results of the study are represented in FIG. 20. 16-hours incubation with 1 mM CHPG led to significant induction of luciferase signal that corresponds to mGluR₅ activation. SIB 1757 administration at both doses (10 and 100 μM) resulted in inhibition of mGluR₅ activity. Both RAHE (SEQ ID NO: 3) and KEDV (SEQ ID NO: 4) peptides reduced the level of CHPG-induced mGluR₅ activation only at a highest dose of 20 μM. The effects of KEDV (SEQ ID NO: 4) on CHPG-induced mGluR₅ activation was significantly more pronounced than in case of RAHE (SEQ ID NO: 3): 65% reduction against 27% respectively.

5.4.2. Part 2. Validation of Peptides' Efficiency at High Doses.

The aim of this study was to test the ability of higher doses of peptides to influence the level of CHPG-induced mGluR₅ activation. Also, the aim was to determine whether studied peptides could act as allosteric antagonists and influence mGluR₅ activity in the absence of CHPG application.

Experimental design:

Control. Non-transfected control, PEI-treated cells

Transfected control

Transfected cells+CHPG

Transfected cells+CHPG+RAHE (SEQ ID NO: 3), 20 μM

Transfected cells+CHPG+RAHE (SEQ ID NO: 3), 100 μM

Transfected cells+CHPG+RAHE (SEQ ID NO: 3), 200 μM

Transfected cells+CHPG+KEDV (SEQ ID NO: 4), 20 μM

Transfected cells+CHPG+KEDV (SEQ ID NO: 4), 100 μM

Transfected cells+CHPG+KEDV (SEQ ID NO: 4), 200 μM

Transfected cells+CHPG+SIB 1757

Transfected cells+CHPG+SIB 1757+RAHE (SEQ ID NO: 3), 200 μM

Transfected cells+CHPG+SIB 1757+KEDV (SEQ ID NO: 4), 200 μM

Transfected cells+RAHE (SEQ ID NO: 3), 20 μM

Transfected cells+RAHE (SEQ ID NO: 3), 200 μM

Transfected cells+KEDV (SEQ ID NO: 4), 20 μM

Transfected cells+KEDV (SEQ ID NO: 4), 200 μM

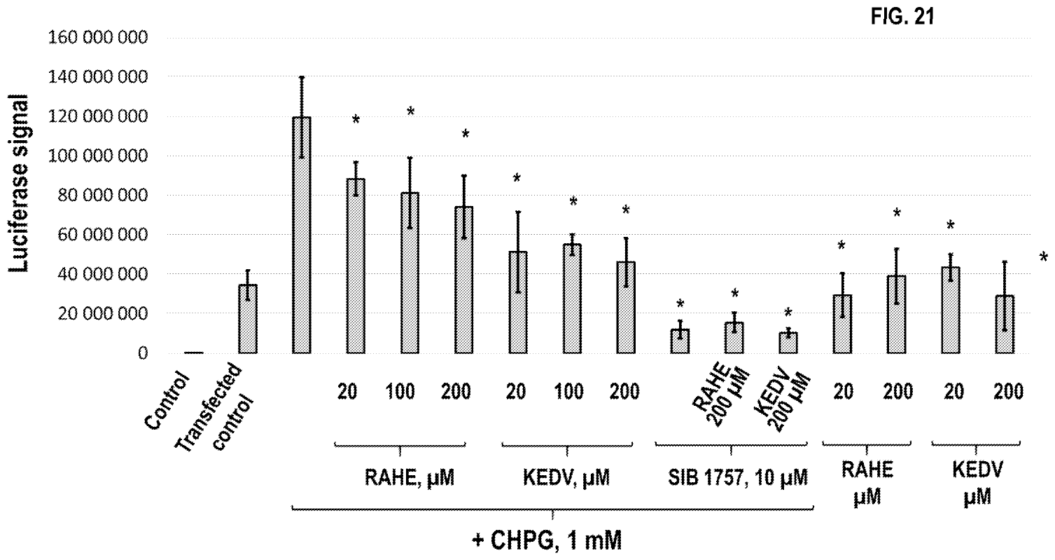

The results of the study are represented in FIG. 21. CHPG, SIB 1757, RAHE (SEQ ID NO: 3) (20 μM) and KEDV (SEQ ID NO: 4) (20 μM) treatment resulted in significant decrease of luciferase signal. Higher doses (100 μM and 200 μM) of both RAHE (SEQ ID NO: 3) and KEDV (SEQ ID NO: 4) peptides did not influence the strength of their inhibitory effect on the level of CHPG-induced mGluR₅ activation. Co-administration of RAHE (SEQ ID NO: 3) or KEDV (SEQ ID NO: 4) peptides with SIB 1757 did not alter the inhibitory effect produced by SIB 1757. Even high doses of both RAHE (SEQ ID NO: 3) and KEDV (SEQ ID NO: 4) peptides did not influence mGluR₅ activity in the absence of CHPG that indicates that both peptides could be classified as negative allosteric modulators

5.5. Conclusions

Both KEDV (SEQ ID NO: 4) and RAHE (SEQ ID NO: 3) peptides act as negative allosteric modulators of mGluR₅ receptor.

The inhibitory effect of KEDV (SEQ ID NO: 4) peptide is more pronounced than those of RAHE (SEQ ID NO: 3).

Higher doses of both RAHE (SEQ ID NO: 3) and KEDV (SEQ ID NO: 4) peptides did not influence the strength of their inhibitory effect on the level of CHPG-induced mGluR5 activation.

According to the results of both studies, 20 μM appears to be the optimal dose for both KEDV (SEQ ID NO: 4) and RAHE (SEQ ID NO: 3) peptides.

### Example 6: Study of the Psychostimulant Potential of KEDV and RAHE Peptides when Administered Intranasally to Wistar Rats

The experiment consisted of two Series. In Series 1, the objective of the study was to assess peak motor activity, endurance, and coordination after i.n. administration of peptides RAHE (SEQ ID NO: 3) and KEDV (SEQ ID NO: 4). Behavioral effects were evaluated in the Locomotor Activity test (LAT), and Beam Walking test (BWT). Also, a relative expression levels of Kcna1, Camk2n1, and EGR2 genes were studied in the frontal cortex of rats receiving 1 mg/kg of KEDV (SEQ ID NO: 4) or RAHE (SEQ ID NO: 3). In Series 2, the objective of the study was to assess effects of i.n. administration of peptide drugs KEDV (SEQ ID NO: 4) and RAHE (SEQ ID NO: 3) on spontaneous motor activity (SMA) in Wistar rats using the ‘Activiscop’ device.

6.1. Materials and Methods

6.1.1. Animals and Treatment

A total of 64 and 31 adult male Wistar rats (250-300 g) were used in Series 1 and Series 2. Animals were housed in the controlled environment, with air conditioning (exchange rate not less than 15 r/h), a 12 h:12 h light-dark cycle (lights on at 09:00 am), air temperature 20-24°±2° C. (possible fluctuations of the limits no more than 2° C. per day), 30-70% humidity. In Series 1 rats were separated into six different groups and the tested substances were administered to the groups as shown in Table 3. In Series 2 rats were divided into four experimental groups, substance and treatment regimen is shown in Table 4.

Caffein (Sodium caffeine benzoate 20%, JSC ‘BZMP’), KEDV (SEQ ID NO: 4) and RAHE (SEQ ID NO: 3) (Lactocore Inc.) solutions for injections were prepared freshly every day in saline. For i.n. administration, a volume of 0.1 ml per kg was applied using automatic pipette, in each nostril. For i.p. a volume of 1 ml per kg of weight was injected with a sterile 1 mL syringe.

TABLE 3
Experimental groups in Series 1.

| | Group | | Behavioral |
|---|---|---|---|
| Group name | size | Test Substance | tests |
| 1. Control | 10 | i.n. administration of a saline | LAT (recording for 48 hours) |
| 2. KEDV 0.1 | 11 | i.n. administration of the KEDV | and BWT |
| mg/kg | | peptide at a dose of 0.1 mg/kg | |
| | | prior to behavior testing | |
| 3. KEDV, 1 | 11 | i.n. administration of the KEDV | |
| mg/kg | | peptide at a dose of 1 mg/kg | |
| | | prior to behavior testing. | |
| 4. RAHE, 0.1 | 11 | i.n. administration OF the RAHE | |
| mg/kg | | peptide at a dose of 0.1 mg/kg | |
| | | before behavior testing. | |
| 5. RAHE, 1 | 11 | i.n. administration OF the RAHE | |
| mg/kg | | peptide at a dose of 1 mg/kg | |
| | | before behavior testing. | |
| 6. Caffeine, 10 | 10 | i.n. administration of caffeine | |
| ma/ka | | at a dose of 10 ma/ka before | |
| | | behavior testina. | |

TABLE 4
Experimental groups in Series 2.

| | Group | | Behavioral |
|---|---|---|---|
| Group name | size | Test Substance | tests |
| 1. Control | 8 | i.n. administration of a | SMA |
| | | saline according to the | |
| | | administration schedule | |
| | | for experimental | |
| 2. Caffeine, 30 | 7 | i.p. administration of | |
| mg/kg | | caffeine at a dose of 30 mg/kg | |
| 3. KEDV, 5 | 8 | i.n. administration of | |
| mg/kg | | KEDV at a dose of 5 mg/kg | |
| 4. RAHE, 5 | 8 | i.n. administration of | |
| mg/kg | | RAHE at a dose of 5 mg/kg | |

In Series 1, at the end of the adaptation period, on the day of the experiment, the animals were injected with the tested substances. The rats were then placed in the LAT unit for 48 hours to assess their peak motor activity. This value was used to calculate the optimal time for administration of the test substances before conducting subsequent behavioral tests. Thus, for all other behavioral tests, the substances were introduced 15 minutes before placing in the experimental setup.

In Series 2, after an adaptation period, the SMA was assessed. Motor activity in animals was assessed for 48 hours. During the first 24 hours, background motor activity was recorded, after which the studied substances were administered. Another 24 hours of motor activity after the peptide administration was recorded. The animals were placed in the installation at 12:00 PM.

6.1.2. Behavioral Tests

In Series 1, the following tests were carried out: Day 1-2—LAT, Day 11—BWT. BWT was video recorded and processed with a video tracking system EthoVision XT, from Noldus. In Series 2, SMA assessment was carries out on Day 1-2. All experimental procedures as well as data analysis were carried out by experimenter blind to treatment.

6.1.2.1. LAT

An apparatus for LAT represents a 32-channel digital actograph that performs synchronous registration of the seismo-acoustic signal of animal motor activity. Each recording chamber (cage) contains only one animal. Animals were placed in standard Type3 cages. All sensors and radioelectronic equipment elements were located on the outside of the cage. The unit was in a separate soundproof room with controlled temperature, humidity, noise, vibration, and light. Animals had unrestricted access to food and water. The experiment (after the administration of test substances, from the beginning of the motor activity) was carried out automatically, without access to the premises by personnel. Experiment started at 4 PM and lasted for 48 hours. Estimated Parameters were: Motor activity (arbitrary units)—measured as fluctuations in the cage that occur when the animal moves (in millivolts). Activity density (fraction)—a numerical value that represents the percentage of minutes of activity in a specific time frame. Each minute of a rat activity were classified as a period of activity or rest. The proportion of such minutes in each group were then calculated for a certain period, called the time frame; in this case a 5-minute period.

6.1.2.2. BWT

This test is used for the assessment of motor coordination, particularly of the hindlimb. Firstly, rats were placed in one corner of the narrow beam and allowed to walk across the narrow beam from one end to the other for at least three times. The narrow beam (RPC OpenScience Ltd) measures 2 cm wide and 165 cm long, with boards located under it to support the animal's limbs during sliding. At the end of the installation was a dark chamber (house), to stimulate the rat to finish the beam walk to the end. The starting point was illuminated with a bright light (100 W), motivating the rats to run to the end of the board into the dark chamber. Experiment was carried out in 3 consecutive days. The first 2 days were training sessions. On the first day animals were placed as follows: 1) in the house for 1 minute; 2) 15 cm from the house; 3) in the house for 1 minute; 4) on the path at distance of ¼ from the house; 5) in the house for 1 minute; 6) at ¼ from the house. On the second day of training, the animals were placed on the installation as follows: 1) in the house for 1 minute; 2) at ¼ from the house; 3) the house for 1 minute; 4) on the path at ½ the length of the path from the house; 5) in the house for 1 minute; 6) on the path at ¾ from the house. During the test day, each animal had three attempts to get from the beginning of the board (the widest part) to the house within 1 minute, after which the animal allowed to stay in the house for 1 minute. The following parameters were evaluated: the number of slips from the board (errors), the total number of steps taken from the starting line to the animal's entry into the dark compartment, and the travel time. For the front and hind limbs, the calculation was carried out separately. The degree of sensorimotor deficit for the front and hind was also calculated using formula: X=Number of Errors/Total number of steps*100%.

6.1.2.3. SMA

Spontaneous motor activity was measured using the Activiscop setup. The animals were kept in home cages with free access to food and water. Motor activity was recorded using an infrared sensor located above each cage. The duration of the experiment was 48 hours the first 24 hours was a background recording. After administering the studied substances, another 24 hours motor activity of rats was recorded. The result obtained are expressed as the number of behavioral acts per minute for each animal.

6.1.3. Animals Euthanasia and Brain Tissue Samples Collection

The next day after the last behavioral test in Series 1, animals from corresponding groups were treated with substances and after 2 h they were sacrificed by decapitation using a guillotine. Brains were removed, washed with cold saline, and placed on cold surface for dissection of the frontal cortex. The brain tissue was snap frozen in liquid nitrogen, and stored at −80° C.

6.1.4. Real-Time PCR of Kcna1, Camk2n1, and EGR2 Genes Expression Levels

Total mRNA was isolated from 30 samples of rat frontal cortex using the ExtractRNA reagent (Eurogen, Russia). Next, the first cDNA chain was synthesized using the Moloney Murine Leukemia Virus Reverse Transcriptase (MMLV RT) kit (Eurogen, Russia). The levels of expression of Kcna1, Camk2n1, EGR2, and Gapdh genes were analyzed by real-time PCR using the qPCRmix-HS SYBR+LowROX kit (Eurogene, Russia) and primers listed in Table 5.

TABLE 5
Primers for real-time PCR analysis.

| | | Primers (forward (fw) | |
|---|---|---|---|
| Gene | | and reverse (rv)) | SEQ ID NO |
| Gapdh | fw | CTTGTGCAGTGCCAGCCTC | SEQ ID NO: 19 |
| | rv | ACCAGCTTCCCATTCTCAGC | SEQ ID NO: 20 |
| Kenai | fw | GACTTCACGGGCACCATTCA | SEQ ID NO: 21 |
| | rv | GAACACCCTTACCAAGCGGA | SEQ ID NO: 22 |
| Camk2n1 | fw | GAGCAAGCGCGTTGTTATTGA | SEQ ID NO: 23 |
| | rv | GTGCTTTCTCCTCCTCATGCT | SEQ ID NO: 24 |
| Egr2 | fw | GGTTTTATGCACCAGCTGCC | SEQ ID NO: 25 |
| | rv | ATGTTGATCATGCCATCTCCAG | SEQ ID NO: 26 |

The results of protein gene expression were normalized for the GAPDH household gene.

6.1.5. Statistical Analysis

Statistical analysis was performed in the statistical programming environment R CRAN as well as the program STATISTICA 10. The differences between the experimental groups with normal distribution were calculated using the analysis of variance (ANOVA) with a post hoc Fisher's LSD test. Where applicable, repeated measurements (repeated-measures ANOVA) with a post hoc Fisher's LSD test was used. The results are presented as the mean±standard error of mean. Differences between groups were considered statistically significant at p<0.05.

6.2. Results

6.2.1. LAT

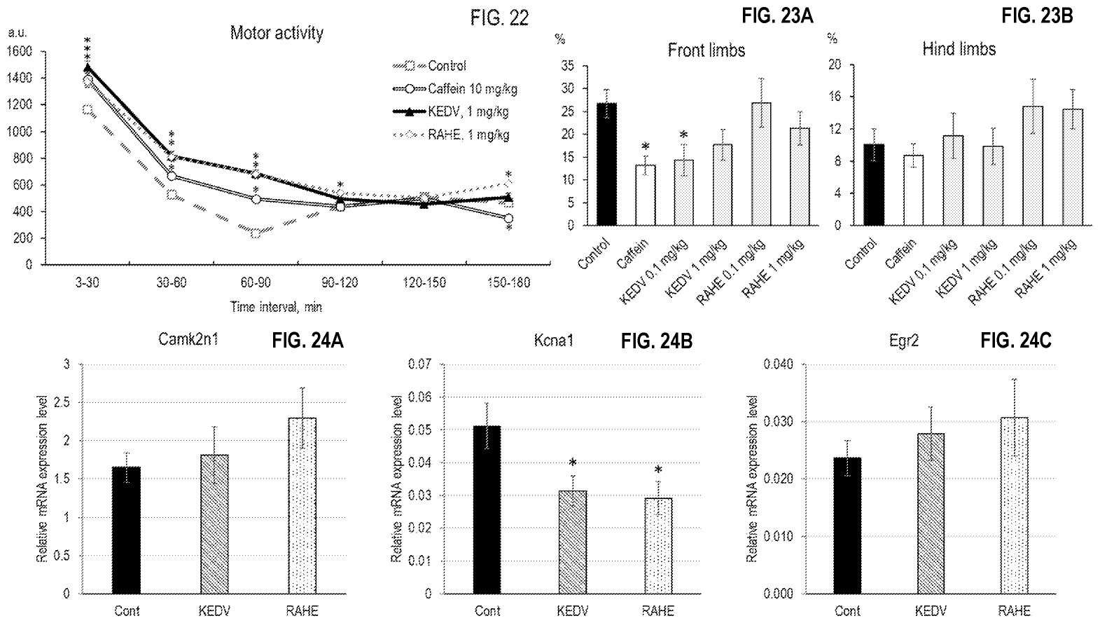

Rat's activity was evaluated at 1-180 minutes interval, after administration of substances. Each minute the rats were classified as being active or resting and the proportion of such minutes in each state were calculated for the 5-minute interval (activity density). KEDV (SEQ ID NO: 4) and RAHE (SEQ ID NO: 3) administration in a dose of 1 mg/kg and caffeine 10 mg/kg led to moderate, unidirectional increases in motor activity, generally indicating a psychostimulant effect of these compounds. For analysis, animal's motor activity was calculated in 30-minute intervals (FIG. 22). Both KEDV (SEQ ID NO: 4) and RAHE (SEQ ID NO: 3) at a dose of 1 mg/kg, as well as Caffeine (10 mg/kg) showed statistically significant increases of motor activity in the intervals of 3-30 minutes, 30-60 minutes, 60-90 minutes (FIG. 22). Differences from the control values were also observed in the 90-120 and 150-180-minute intervals in the RAHE (SEQ ID NO: 3) group (FIG. 22). Animals receiving caffeine showed marked sedation in the interval of 150-180 minutes, and no motor depression after KEDV (SEQ ID NO: 4) and RAHE (SEQ ID NO: 3) was observed (FIG. 22).

6.2.2. BWT

Treatment with caffein (10 mg/kg) and KEDV (SEQ ID NO: 4) (0.1 mg/kg) resulted in reduced severity of sensorimotor deficits in the front paws (FIG. 23A). No effect of treatments was observed in the hind paws (FIG. 23B).

6.2.3. Kcna1, Camk2n1, and EGR2 mRNA Relative Expression in the Frontal Cortex of Rats

The obtained results revealed that KEDV (SEQ ID NO: 4) and RAHE (SEQ ID NO: 3) at a dose of 1 mg/kg did not affect expression levels of Camk2n1 and EGR2 genes (FIGS. 24A, 24C). At the same time, both KEDV (SEQ ID NO: 4) and RAHE (SEQ ID NO: 3) led to a statistically significant decrease in the expression of Kcna1 gene (FIG. 24B).

6.2.4. SMA

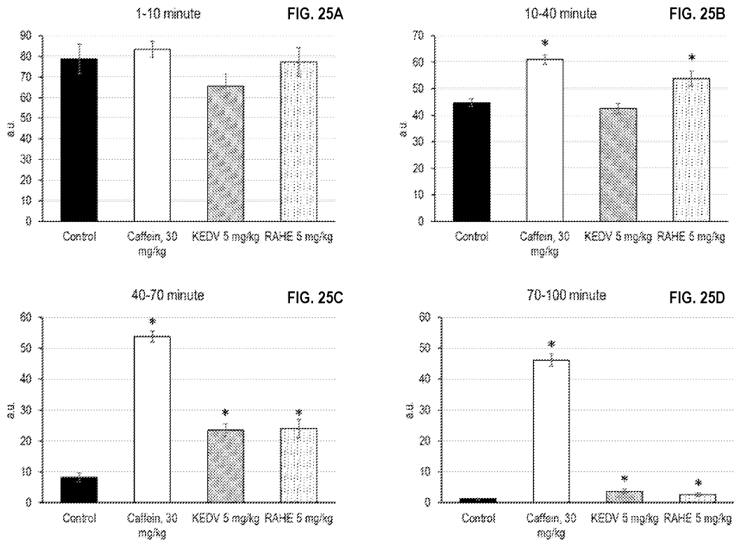

A mild stimulating effect was shown after i.n. administration of KEDV (SEQ ID NO: 4) in the 40-100-minute interval, and RAHE (SEQ ID NO: 3) in the 10-100-minute interval (FIGS. 25A, 25B, 25C, and 25D). At the same time, no sedation was observed in any of the peptide-treated groups during the experiment. Caffeine at a dose of 30 mg/kg also led to a significant increase in the number of motor acts in animals at these time intervals.

6.3. Discussion

The aim of this study was to evaluate the possible stimulating effect of KEDV (SEQ ID NO: 4) and RAHE (SEQ ID NO: 3) peptides. For this purpose, two series of experiments were conducted to study spontaneous motor activity in home cages, i.e. in familiar conditions that are not stressful for animals. In Series 1, registration of a seismo-acoustic signal received from moving animals was carried out (LAT). This approach is highly accurate and sensitive to any changes in the activity of animals. In Series 2, registered changes of the motor activity were obtained using infrared sensors (SMA test).

The stimulating effect of caffein was observed in both series. In Series 1, increased motor activity after caffein administration was followed by marked decrement of locomotion at the 180-minute interval. Increased locomotor activity after treatment with KEDV (SEQ ID NO: 4) and RAHE (SEQ ID NO: 3) peptides was observed in both series, and the duration of the stimulating effect of these tests was at least 90-100 minutes, without sedation at later time intervals. In Series 1, the changes of motor activity after RAHE (SEQ ID NO: 3) administration at a dose of 1 mg/kg maintained for up to 180 minutes. Stimulating effects were observed after the administration of the peptides at a dose of 1 and 5 mg/kg, but not 0.1 mg/kg.

In the coordination test (BWT), a decrease in the severity of sensorimotor deficits for the front limbs was shown for the caffeine and KEDV (SEQ ID NO: 4) 0.1 mg/kg groups. A decrease in these values can be interpreted as an improvement in animal coordination.

To evaluate the potential mechanism of action of the peptides KEDV (SEQ ID NO: 4) and RAHE (SEQ ID NO: 3), expression markers affecting the mGluR5-dependent signaling cascade were analyzed by real-time PCR and the Kcnal, Camk2n1, and EGR2 were chosen as target genes. The analyzed genes were chosen based on literature data, which showed changes in the levels of expression in vivo in response to the introduction of known mGluR5 antagonists, as well as considering their functional significance. Thus, in schizophrenia and several other conditions, the expression of EGR2 is increased (Cheng et al. (2012) Genetic and functional analyses of early growth response (EGR) family genes in schizophrenia. Prog Neuropsychopharmacol Biol Psychiatry 39(1):149-155), and mGluR5 antagonists can potentially normalize the condition. The product of the Camk2n1 gene-CaM kinase II inhibitor alpha as a regulator of the calmodulin-dependent MAPK cascade can be involved in the regulation of synaptic plasticity, and the effect of mGluR5 antagonists on it can explain their effects on learning and memory (Simonyi et al. (2010) Metabotropic glutamate receptor subtype 5 antagonism in learning and memory. Eur J Pharmacol 639(1-3):17-25).

An important contribution to the excitability of nervous tissue is made by potassium channels, including voltage-dependent Kv1. Activation of potassium channels generally leads to hyperpolarization of the cytoplasmic membrane of excitable cells, which prevents the transmission of a nerve impulse. In contrast, a decrease in the activity or expression of these channels contributes to depolarization and consequently facilitates further transmission of the action potential. For example, a decrease in the expression of the Kcna1 gene (the Kv1.1 potassium channel gene) in response to the introduction of mGluR5 antagonists can be expressed in a change in the level of excitability of neurons in the frontal cortex and lead to an increase in motor activity (Homayoun et al. (2006) Bursting of prefrontal cortex neurons in awake rats is regulated by metabotropic glutamate 5 (mGlu5) receptors: rate-dependent influence and interaction with NMDA receptors. Cereb Cortex 16(1): 93-105). The decrease in Kcna1 expression presumably explains the ability of mGluR5 antagonists to reduce the spontaneous burst activity of cortical neurons and the random activity of dendritic spikes (Homayoun et al. (2006); Gass et al. (2008) Transcriptional profiling of the rat frontal cortex following administration of the mGlu5 receptor antagonists MPEP and MTEP. Eur J Pharmacol 584 (2-3): 253-262), which may also be accompanied by changes in motor activity in general.

According to the data obtained, a statistically significant decrease in the expression level was observed specifically for the Kcnal gene after administration of both KEDV (SEQ ID NO: 4) and RAHE (SEQ ID NO: 3). The effect of peptides on the level of Kcnal expression is similar to those effects of known mGluR5 antagonists MTEP and MPEP (Gass et al. (2008)), which may potentially indicate the action of peptides by a similar mechanism.

6.4. Conclusions

Intranasal administration of KEDV (SEQ ID NO: 4) and RAHE (SEQ ID NO: 3) at a dose of 1 mg/kg led to a marked stimulating effect on locomotion, comparable to that of 10 mg/kg caffeine.

The stimulating effect after intranasal administration of KEDV (SEQ ID NO: 4) and RAHE (SEQ ID NO: 3) lasted about 90 minutes and 180 minutes, respectively. No sedation was observed in peptide-treated groups during the observations.

Intranasal administration of small doses (0.1 mg/kg) of KEDV (SEQ ID NO: 4) and RAHE (SEQ ID NO: 3) did not significantly affect the spontaneous motor activity of rats.

In the Beam Walking test, administration with 10 mg/kg caffeine and 0.1 mg/kg KEDV (SEQ ID NO: 4) resulted in abolished sensorimotor deficits in the front limbs, which may indicate improved coordination.

Treatment with KEDV (SEQ ID NO: 4) and RAHE (SEQ ID NO: 3) at a dose of 1 mg/kg resulted in decreased expression levels of Kcnal mRNA in the frontal cortex of rats.

### Example 7: The Study of the Effects of Intranasal Administration of KEDV and RAHE on the Behavioral and Endocrine Parameters of Rats in Acute Foot Shock Stress Model

The aim of the study is to investigate the potential antidepressant-like and anxiolytic-like effects of intranasal administration of KEDV (SEQ ID NO: 4) and RAHE (SEQ ID NO: 3) on the behavioral and endocrine parameters of rats in the Acute Foot Shock (AFS) model.

7.1. Materials and Methods.

7.1.1. Animals and Treatment

The study was performed on 63 adult male Wistar rats 240-280 g, (average weight 260 g) from the “Collection of laboratory mammals of different taxonomic affiliations” of the IPh RAS, supported by the program of bioresource collections of the FANO of Russia, kept in standard conditions. All procedures involving animals were conducted in accordance with the European (Directive 2010/63/EU of the European Parliament and of the Council of 22 Sep. 2010 on the protection of animals used for scientific purposes) and the Russian (“GOST 33216-2014 Guidelines for the maintenance and care of laboratory animals. Rules for the maintenance and care of laboratory rodents and rabbits”) bioethical guidelines.

KEDV (SEQ ID NO: 4) and RAHE (SEQ ID NO: 3) (Lactocore Inc.) were administered intranasally (i.n), daily for 10 days at a dose of 1 mg/kg and 5 mg/kg. Peptides were prepared freshly each day in saline. The solution for i.n. administration of 5 mg/kg in a volume of 15 μl (single administration) contained 1.3 mg KEDV (SEQ ID NO: 4) or RAHE (SEQ ID NO: 3), and a solution for intranasal administration of 1 mg/kg in a volume of 10 μl (single administration) contained 0.26 mg KEDV (SEQ ID NO: 4) or RAHE (SEQ ID NO: 3).

The tetracyclic antidepressant Maprotiline (MAP, “Lyudiomil”), was used as a drug of comparison. Animals received a daily intraperitoneal injection (i.p) of MAP for ten days (M9651, Merck, in a dose of 4.5 mg/kg, dissolved in saline, 250 μl per administration).

7.1.2. Acute Foot Shock (AFS) Stress Model

The classical paradigm of “learned helplessness” (LH) was used as an experimental model of depressive-like state in rats (Seligman et al. (1975). Learned helplessness in the rat. J. Comp. Physiol. Psychol., 88(2), 534.]. To develop LH, rats were subjected to uncontrolled unavoidable aversive stress—acute foot shock (AFS) (electro cutaneous irritation). The animals were stimulated with electric current (1 mA, 1 Hz, 15 sec) in a closed space of a 13×16×26 cm-sized cage with a conductive floor using an interval of different durations between applying current to the chamber floor so that each rat received 60 stimulations within an hour, which resulted in the development of a persistent depressive-like state. Stimulation was performed automatically using a software randomizer.

7.1.3. Behavioral Tests

The schedule of behavioral tests is shown in Table 6.

7.1.3.1. Open Field (OF) Test

The OF test is a classic method for assessing the level of motor activity and exploratory behavior of rodents in new stressogenic conditions. The test was performed in a cage of 90×90×45 cm without a roof, the floor of which was laid out on squares 15×15 cm and lit from above by a 60 W lamp. On the 5th day after stressing in the LH model, the rat was placed in the center of the OF. The following parameters were measured for 5 minutes: latency to start of moving, the number of crossed squares, the duration of rears and freezing.

7.1.3.2. Elevated Plus Maze (EPM) Test

EPM testing allows characterizing the behavior of rodents under the variable stress conditions, which makes it possible to assess the level of animal anxiety and anxiolytic effects of drugs. On the 6th day after the experimental exposure, the rats were tested one at a time for 5 min in the EPM installation, located at a height of 75 cm above the floor, and consisting of 2 open illuminated and 2 closed arms with exits. The time spent by the animal inside and outside the closed arms (in the open arms and in the center), the number of transitions between the arms, the number of stretch-attended postures were evaluated. Usually, animals tend to stay in the closed arms, anxiolytic treatment results in an increment of the time spent on the open arms of the maze (Pellow et al. (1985). Validation of open: closed arm entries in an elevated plus-maze as a measure of anxiety in the rat. J. Neurosci. Methods, 14(3), 149-167; Walf et al. (2007). The use of the elevated plus maze as an assay of anxiety-related behavior in rodents. Nat. Protoc., 2(2), 322-328).

7.1.4. Dexamethasone Test (DXMT)

Evaluation of the stress-evoked release of glucocorticoid hormones (corticosterone, an analog of cortisol in humans) and its suppression by the introduction of synthetic glucocorticosteroid was performed on the 9-10th day after the development of LH in a two-day dexamethasone test according to the scheme, taking into account the specificity of the circadian rhythm of HPA axis function in rats (Zhukov (1993). The dexamethasone suppression test in genetically different rats exposed to inescapable and escapable electric shocks. Psychoneuroendocrinology, 18(7), 467-474.].

On the first day of the test (DXMT1) at 10:00 AM, animals were injected (i.p.) with saline, then at 04:00 PM on the same day peripheral blood samples were taken to determine the basal level of the hormone, which caused stress, and 30 minutes after taking the rat from the cage and receiving initial sample, re-taken blood to measure hormone stress level.

To study the sensitivity of the HPA system to feedback signals, rats were injected with dexamethasone (DXM, 10 μg/kg, i.p.) the next day (DXMT2) at 10 AM. The procedure for taking blood from the tail vein was repeated after 6 and 6.5 hours. The corticosterone content was determined by enzyme-linked immunosorbent assay with reagent kits “Corticosterone-ELISA” (“Hema”, RF) in two parallel samples.

The experimental data were processed by calculating the mean and standard error of the mean in the studied subgroups of animals, n=9 for each point.

7.1.5. Experimental Design

Laboratory rats were divided into 7 experimental groups of 9 animals each (Table 6):

(1) “Control”—control group had a 10-day daily i.n. administration of the saline. (2) “AFS+veh”—a group of animals subjected to stress and received vehicle (saline). (3) “AFS+KEDV, 5 mg/kg” and (4) “AFS+KEDV, 1 mg/kg”—after stress rats received daily i.n. injections of KEDV at a dose of 5 or 1 mg/kg for 10 days. (5) “AFS+RAHE, 5 mg/kg” and (6) “AFS+RAHE, 1 mg/kg”—after stress rats received daily i.n. injections of RAHE at a dose of 5 or 1 mg/kg for 10 days. (7) “AFS+MAP”—after stress rats received daily i.p. injections of comparison drug Maprotiline (MAP) at a dose of 4.5 for 10 days.

TABLE 6
Experimental design, drug administration and schedule of behavioral testing.

| | | | Day of Experiment | | | | | | | | | |
|---|---|---|---|---|---|---|---|---|---|---|---|---|
| # | Treatment | | 1 | 2 | 3 | 4 | 5 | 6 | 7 | 8 | 9 | 10 |
| 1 | saline | non- | saline | —//— | —//— | —//— | —//— | —//— | —//— | —//— | —//— | —//— |
| | | stressed | | | | | OF | EPM | | | DXMT1 | DXMT2 |
| 2 | saline | AFS | saline | —//— | —//— | —//— | | | —//— | —//— | | |
| 3 | KEDV | AFS | KEDV, 5 | —//— | —//— | —//— | | | —//— | —//— | | |
| | | | mg/kg | | | | | | | | | |
| 4 | KEDV | AFS | KEDV, 1 | —//— | —//— | —//— | | | —//— | —//— | | |
| | | | mg/kg | | | | | | | | | |
| 5 | RAHE | AFS | RAHE, 5 | —//— | —//— | —//— | | | —//— | —//— | | |
| | | | mg/kg | | | | | | | | | |
| 6 | RAHE | AFS | RAHE, 1 | —//— | —//— | —//— | | | —//— | —//— | | |
| | | | mg/kg | | | | | | | | | |
| 7 | MAP | AFS | Map | —//— | —//— | —//— | | | —//— | —//— | | |

7.1.6. Statistical Analysis

For a normally distributed data, a one-way analysis of variance (ANOVA) using the post-hoc Tukey test was used. Categorial data was assessed using Median test and X² test with Yates' correction. The significance threshold was set at p<0.05. Data are presented as mean±standard error of the mean, or as a stacked histogram.

7.2. Results

7.2.1. OF

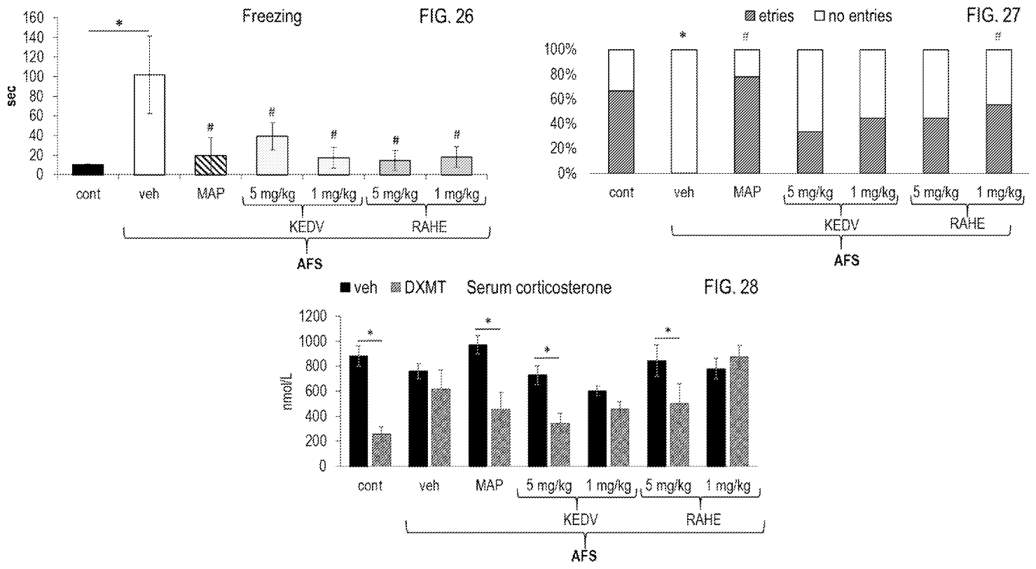

AFS resulted in a longer duration of freezing in comparison with the control unstressed animals, which suggests a higher anxiety in AFS group (FIG. 26). KEDV (SEQ ID NO: 4) and RAHE (SEQ ID NO: 3) treatment in a dose of 1 and 5 mg/kg as well as MAP administration resulted in significant reduction of freezing behavior in comparison with stressed saline-treated animals (FIG. 26). This result suggests normalization of emotional state in animals receiving peptides, similar to those, observed after treatment with tricyclic antidepressant.

7.2.2. EPM

To estimate the time spent on the open arms of the maze, the median test was used, which showed that the median values between the studied groups were different (Table 7). Values above the overall median (0 s) indicate that the animal entered the open arms of the maze. Values equal to the median indicate that the animal did not leave the dark arms of the EPM. In the control group, about 67% of animals visited the open arms of the maze, while in the “AFS+veh” group none of the animals left the dark arms of the maze (FIG. 27). These differences are statistically significant (p=0.012, X2 with Yates' correction). 78% of the animals treated with MAP, and 56% treated with RAHE (SEQ ID NO: 3) at a dose of 1 mg/kg, went to the open arms of the maze. This is significantly more than in the stressed group of rats treated with the vehicle (p=0.004 and p=0.035, X2 with Yates' correction). The animals in the 5 mg/kg RAHE (SEQ ID NO: 3) and 1 mg/kg KEDV (SEQ ID NO: 4) groups went to the open arms more often (44% of cases) than the animals of the group LH, but only at the trend level. They did not differ from this parameter from the control group (FIG. 27).

TABLE 7
Time spent in the open arms of the EPM: the result of the Median test.
Median Test, Overall Median (OM) = 0.00; Chi-Square = 13.80, df = 6 p = 0.03

| | AFS | | | | | | | |
|---|---|---|---|---|---|---|---|---|
| | | | KEDV | RAHE | KEDV | RAHE | | |
| | Cont | veh | 5 mg/kg | 5 mg/kg | 1 mg/kg | 1 mg/kg | MAP | Total |
| <=Median: | 3.00 | 9.00 | 6.00 | 5.00 | 5.00 | 4.00 | 2.00 | 34.00 |
| observed | | | | | | | | |
| expected | 4.86 | 4.86 | 4.86 | 4.86 | 4.86 | 4.86 | 4.86 | |
| obs. − exp. | −1.86 | 4.14 | 1.14 | 0.14 | 0.14 | −0.86 | −2.86 | |
| >Median: | 6.00 | 0.00 | 3.00 | 4.00 | 4.00 | 5.00 | 7.00 | 29.00 |
| observed | | | | | | | | |
| expected | 4.14 | 4.14 | 4.14 | 4.14 | 4.14 | 4.14 | 4.14 | |
| obs. − exp. | 1.86 | −4.14 | −1.14 | −0.14 | −0.14 | 0.86 | 2.86 | |
| Total: | 9.00 | 9.00 | 9.00 | 9.00 | 9.00 | 9.00 | 9.00 | 63.00 |
| observed | | | | | | | | |

7.2.3. DXMT

Injection of DXM at a dose of 10 μg/kg should normally be accompanied by a decrease in the concentration of blood corticosterone (CS), as it is demonstrated in the control group of animals (FIG. 5). At the same time, a disruption of the regulation of the hypothalamic-pituitary-adrenal (HPA) axis in stressed groups of rats were noted. In “AFS+veh” group DXM failed to reduce stress CS levels in serum, in comparison with 70% suppression in control rats and more than 50% suppression in the group that was administered with Map (FIG. 28). Chronic intranasal administration of KEDV (SEQ ID NO: 4) and RAHE (SEQ ID NO: 3) peptides at a dose of 5 mg/kg also resulted in a significant decrease in CS concentration in response to DXMT (FIG. 28). This may indicate normalization of the HPA regulation in animals after stress and peptide treatment.

7.3. Discussion

Acute foot shock stress model resulted in enhanced freezing in the OF test, pronounced open-arms avoidance in the EPM, and a disrupted regulation of HPA axis in rats. These effects suggest an increment of anxiety-like behavior and emotionality, as well as endocrine system malfunctions, which often accompany depressive disorders.

Repeated intranasal administration of KEDV (SEQ ID NO: 4) and −15 at doses of 1 and 5 mg/kg reduced stress-evoked freezing in rats in the OF test, which suggest normalization of emotionality and anxiolysis in animals. In the EPM, both KEDV (SEQ ID NO: 4) and RAHE (SEQ ID NO: 3) increased the number of exits to the open arms of the maze, thus only RAHE (SEQ ID NO: 3) at a dose of 1 mg/kg significantly improved open arms preference in rats. This effect may propose a decreased anxiety in animals treated with the studied peptides. Moreover, both KEDV (SEQ ID NO: 4) and RAHE (SEQ ID NO: 3) restored DXM-evoked suppression of corticosterone in plasma. This may suggest normalization of HPA axis regulation in peptide-treated groups. KEDV (SEQ ID NO: 4) and RAHE (SEQ ID NO: 3) effects were similar to those observed after MAP (Maprotiline) treatment.

Thus, the results of the study indicate that ten-day intranasal administration of the peptide drugs KEDV (SEQ ID NO: 4) and RAHE (SEQ ID NO: 3) has a moderate antidepressant-like and anxiolytic-like effects and is able to correct post-stress behavioral disorders in the stress models.

7.4. Conclusions

AFS resulted in anxiety and depressive-like state in animals.

Intranasal administration of KEDV (SEQ ID NO: 4) and RAHE (SEQ ID NO: 3) for 10 days after AFS resulted in moderate anxiolytic-like and antidepressant-like effects.

KEDV (SEQ ID NO: 4) and RAHE (SEQ ID NO: 3) effects were similar to those observed in animals treated with tricyclic antidepressant Maprotiline (MAP), which may propose a potent implication in treatment of stress-evoked disorders.

### Example 8: Testing of Potential Glutamate Receptor mGluR5 Peptide Antagonists KEDV and RAHE Using Calcium-Flux Imaging

The aim of the study was to evaluate the influence of potential mGluR5 antagonists—KEDV (SEQ ID NO: 4) and RAHE (SEQ ID NO: 3)—on sodium glutamate evoked [Ca²+] responses in CHO-mGluR5 cells.

8.1. Materials and Methods

8.1.1. Model

CHO-mGluR5 cells with tetracycline-induced stable expression of human mGluR5.

8.1.2. Experimental Procedures

The CHO cell line stably expressing human mGluR5 was generated using T-Rex System (Thermo Fisher Scientific Inc., Waltham, Mass., USA) according to the manufacturer's' instructions. Briefly, cDNA encoding human mGluR5 was subcloned into inducible expression vector pcDNA4/TO and was transfected into CHO cells carrying regulatory vector pcDNA6/TR that expresses the tetracycline repressor. After 2 weeks of selection using Blasticidin (5 lg/ml) and zeocin (250 lg/ml), pools of cells were screened for the expression of mGluR5 in the agonist-induced [Ca²+] uptake assay. Positive cells were expanded and used. mGluR5 expression was induced by adding tetracycline (up to 1 lg/ml) 16 h before testing.

Fluorescent assays were performed using NOVOstar (BMG LABTECH, Ortenberg, Germany). CHO-mGluR5 cells were seeded into black-walled clear-bottomed 96-well plates at a density of 75,000 cells per well (complete media without antibiotics and containing 1 lg/ml of tetracycline to induce receptor expression) and were cultured overnight at 37° C. The cells were then loaded with the cytoplasmic calcium indicator Fluo-4AM using Fluo-4 Direct™ Calcium Assay Kits (Thermo Fisher Scientific Inc., Waltham, Mass., USA), and incubated in the dark at 37° C. for 60 min, and then at 25° C. for 60 min. The buffer alone (control) or the buffer containing different concentrations of KEDV (SEQ ID NO: 4) peptide, RAHE (SEQ ID NO: 3) peptide (0.02, 2, 20 and 200 μM) or MPEP (0.1, 1, 10, 100 μM) (Sigma Aldrich, USA) were added to the cells. After a 3-min incubation at 37° C., changes in cell fluorescence (lex=485 nM, lem=520 nM) were monitored before and after the addition of the mGLuR5 agonist (1 mM sodium glutamate—Glu-Na). The measurements were performed at pH 7.4 and 37° C.

8.2. Results

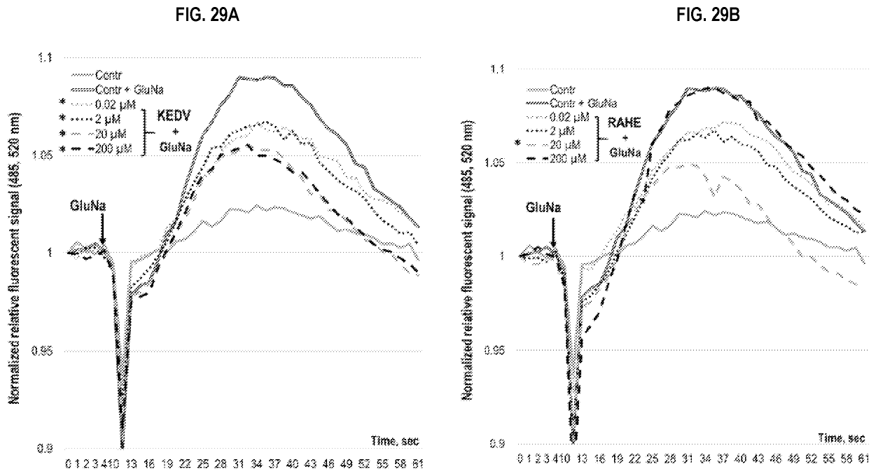

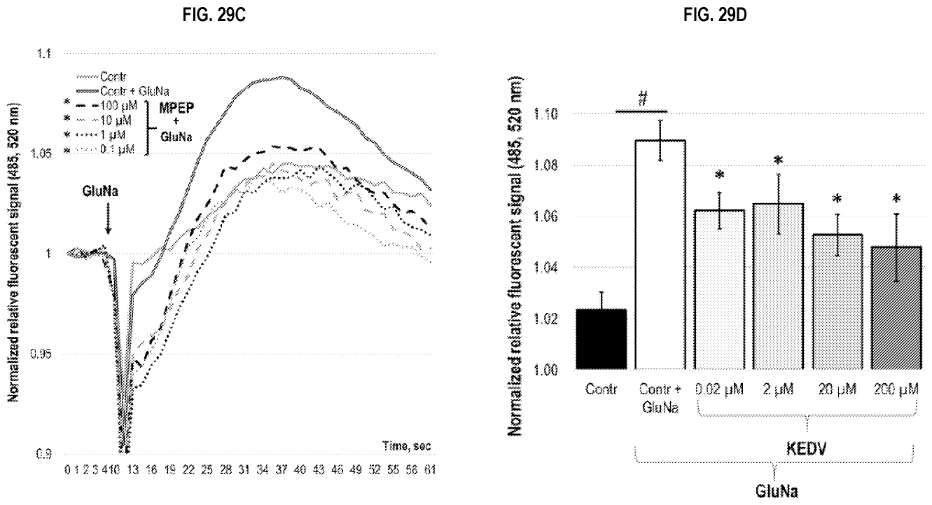

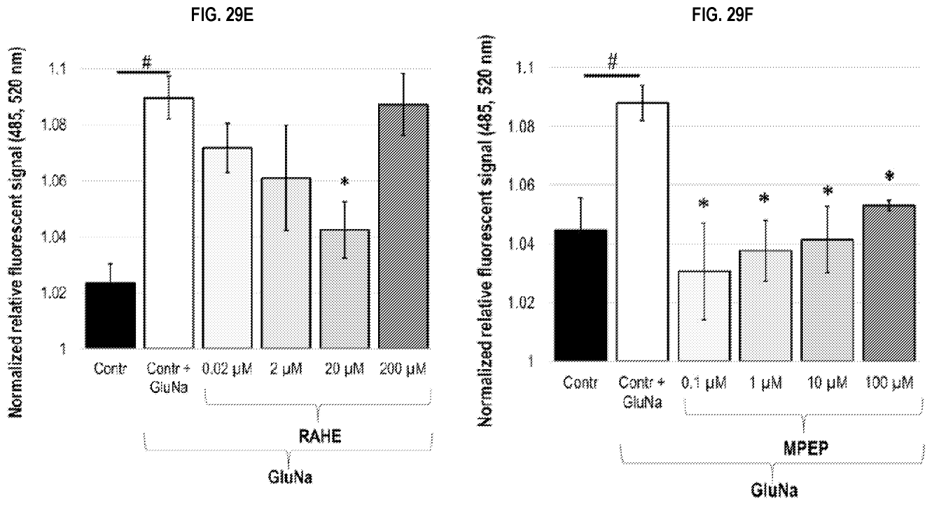

The results of the measurements of [Ca²+] responses of CHO-mGluR5 cells to 1 mM sodium glutamate (GluNa) in the absence or presence of antagonists in different concentrations are represented in FIGS. 29A, 29B, and 29C. The inhibitory effects of KEDV (SEQ ID NO: 4) and RAHE (SEQ ID NO: 3) peptides on intracellular [Ca²+] levels on the moment of peak CHO-mGluR5 cells activation by 1 mM Glu-Na (27 second after Glu-Na application) are represented in FIGS. 29D, 29E, and 29F.

KEDV (SEQ ID NO: 4) was potent to significantly abolish [Ca²+] currents at whole range of concentrations applied FIGS. 29A and 29D). RAHE (SEQ ID NO: 3) only at a concentration of 20 μM was able to reduce [Ca²+] responses to GluNa (FIGS. 29B and 29E).

To sum up, an application of 1 mM GluNa to CHO-mGluR5 cells leads to elevated intracellular [Ca²+] levels as compared to the cells treated with the buffer. Pre-treatment of CHO-mGluR5 cells with commercially available antagonist MPEP is resulted in abolished GluNa-induced activation. Both tested peptides, KEDV (SEQ ID NO: 4) and RAHE (SEQ ID NO: 3), also demonstrated effects typical for mGluR5 receptor antagonists: they reduce Glu-Na-induced cytoplasmic [Ca²+] levels in CHO-mGluR5 cells. RAHE (SEQ ID NO: 3) peptide's effects were observed in 20 μM concentration. KEDV (SEQ ID NO: 4) peptide acted at more physiologically relevant concentrations (starting from 0.02 μM).

8.3. Conclusions

Both KEDV (SEQ ID NO: 4) and RAHE (SEQ ID NO: 3) demonstrate effects typical for mGluR5 receptor antagonists: they reduce Glu-Na-induced cytoplasmic [Ca²+] levels in CHO-mGluR5 cells.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A composition comprising a synthetic neuromodulatory peptide, the neuromodulatory peptide being defined by the general Formula I:
R₁R₂R₃R₄ (I)
wherein:
at least one of R₁-R₄ is hydrophobic and at least one of R₁-R₄ is polar or charged;
none of R₁-R₄ is selected from L, M, I, T, C, P, N, Q, F, Y, and W; and
the peptide modulates the mGluR₅ receptor (GRM5).

2. The composition of claim 1, wherein R₁ is R or K.

3. The composition of claim 1, wherein R₁ is D or E.

4. The composition of claim 1, wherein R₁ is S.

5. The composition of claim 1, wherein R₂ is hydrophilic neutral.

6. The composition of claim 1, wherein R₂ is negatively charged hydrophilic.

7. The composition of claim 1, wherein R₂ is hydrophobic neutral.

8. The composition of claim 1, wherein R₂ is A, S, E, or D.

9. The composition of claim 1, wherein R₂ is A.

10. The composition of claim 1, wherein R₂ is S.

11. The composition of claim 1, wherein R₂ is E or D.

12. The composition of claim 1 wherein R₃ is G, H, S, or D.

13. The composition of claim 12, wherein R₃ is G.

14. The composition of claim 12, wherein R₃ is H.

15. The composition of claim 12, wherein R₃ is S.

16. The composition of claim 12, wherein R₃ is D.

17. The composition of claim 1, wherein R₄ is S, H, V or E.

18. The composition of claim 17, wherein R₄ is S.

19. The composition of claim 17, wherein R₄ is H.

20. The composition of claim 17, wherein R₄ is V.

21. The composition of claim 17, wherein R₄ is E.

22. The composition of claim 1, wherein:
R₁ is D;
R₂ is S;
R₃ is G; and
R₄ is H.

23. The composition of claim 1, wherein:
R₁ is R;
R₂ is A;
R₃ is H; and
R₄ is E.

24. The composition of claim 1, wherein:
R₁ is K;
R₂ is E;
R₃ is D; and
R₄ is V.

25. The composition of claim 1, wherein:
R₁ is A;
R₂ is G;
R₃ is A; and
R₄ is S.

26. The composition of claim 1, wherein:
each of R₁, R₂, and R₃ is a hydrophobic, aliphatic amino acid; and
R₄ is a polar and neutral of charge hydrophilic amino acid.

27. The composition of claim 1, wherein:
R₁ is a polar and negatively charged hydrophilic amino acid;
R₂ is a polar and neutral of charge hydrophilic amino acid;
R₃ is a hydrophobic, aliphatic amino acid; and
R₄ is an aromatic, polar and positively charged hydrophilic amino acid.

28. The composition of claim 27, wherein:
R₁ is D;
R₂ is S;
R₃ is G, A, or V; and
R₄ is H.

29. The composition of claim 1, wherein:
R₁ is a polar and positively charged hydrophilic amino acid;
R₂ is a hydrophobic, aliphatic amino acid;
R₃ is an aromatic, polar and positively charged hydrophilic amino acid; and
R₄ is a polar and negatively charged hydrophilic amino acid.

30. The composition of claim 29, wherein:
R₁ is R or K;
R₂ is G, A, or V;
R₃ is H; and
R₄ is D or E.

31. The composition of claim 1, wherein:
R₁ is a polar and positively charged hydrophilic amino acid;
R₂ is a polar and negatively charged hydrophilic amino acid;
R₃ is a polar and negatively charged hydrophilic amino acid; and
R₄ is a hydrophobic, aliphatic amino acid.

32. The composition of claim 31, wherein:
R₁ is R or K;
R₂ is D or E;
R₃ is D or E; and
R₄ is G, A, or V.

33. The composition of claim 1, wherein:
R₁ is selected from R, K, D, A, and E;
R₂ is selected from A, S, G, D, and E;
R₃ is selected from S, G, D, E, A, and H; and
R₄ is selected from S, H, V, and E.

34. The composition of claim 33, wherein:
R₁ is D;
R₂ is S;
R₃ is G; and
R₄ is H.

35. The composition of claim 33, wherein:
R₁ is R;
R₂ is A;
R₃ is H; and
R₄ is E.

36. The composition of claim 33, wherein:
R₁ is K;
R₂ is E;
R₃ is D; and
R₄ is V.

37. The composition of claim 33, wherein:
R₁ is A;
R₂ is G;
R₃ is A; and
R₄ is S.

38. A composition comprising a synthetic neuromodulatory peptide, the neuromodulatory peptide being defined by the general formula II:
R₁R₂R₃R₄ (II)
wherein:
R₁ is selected from the amino acids that are non-hydrophobic and not aromatic; the amino acids that contain a full positive charge on a side chain; the amino acids that contain a full negative charge on a side chain; and the amino acids that are non-charged and contain no more than 5 atoms in the side chain;
R₂ is selected from the amino acids that are non-charged and containing no more than 5 atoms in a side chain, and the amino acids that contain a full negative charge on a side chain;
R₃ is selected from the amino acids that are non-charged and contain no more than 5 atoms in a side chain; the amino acids that contain a full negative charge on a side chain; and the amino acids that are aromatic non-hydrophobic; and
R₄ is selected from the amino acids that do not include W, Y, F, P, I.

39. The composition of claim 38, wherein:
R₁ is selected from A, R, K, D, E, Q, N, S, T, C, and M;
R₂ is selected from A, S, G, D, and E;
R₃ is selected from S, A, G, D, E, and H; and
R₄ is selected from S, H, V, and E.

40. The composition of claim 38, wherein:
R₁ is selected from R, K, D, A, and E;
R₂ is selected from A, S, G, D, and E;
R₃ is selected from S, G, D, E, A, and H; and
R₄ is selected from S, H, V, and E.

41. The composition of claim 40, wherein:
R₁ is D;
R₂ is S;
R₃ is G; and
R₄ is H.

42. The composition of claim 40, wherein:
R₁ is R;
R₂ is A;
R₃ is H; and
R₄ is E.

43. The composition of claim 40, wherein:
R₁ is K;
R₂ is E;
R₃ is D; and
R₄ is V.

44. The composition of claim 40, wherein:
R₁ is A;
R₂ is G;
R₃ is A; and
R₄ is S.

45. The composition of claim 38, wherein R₁ is R, K, D, E, S or A.

46. The composition of claim 38, wherein R₁ is R.

47. The composition of claim 38, wherein R₁ is D.

48. The composition of claim 38, wherein R₁ is K.

49. The composition of claim 38, wherein R₁ is A.

50. The composition of claim 38, wherein R₂ is S.

51. The composition of claim 38, wherein R₂ is A.

52. The composition of claim 38, wherein R₂ is G.

53. The composition of claim 38, wherein R₂ is E.

54. The composition of claim 38, wherein R₃ is G.

55. The composition of claim 38, wherein R₃ is H or D.

56. The composition of claim 38, wherein R₃ is A.

57. The composition of any one of the above claims, wherein the neuromodulatory peptide consists of amino acids that do not include proline.

58. The composition of any one of claims 1-57, wherein the peptide is optionally chemically modified.

59. The composition of claim 58, wherein the chemical modification is selected from amidation, methylation, and acetylation of one or more of R₁, R₂, R₃, and R₄.

60. The composition of claim 58, wherein the chemical modification is selected from addition of formyl, pyroglutamyl (pGlu), a fatty acid, urea, carbamate, sulfonamide, alkylamine, or any combination thereof, to one or more of R₁, R₂, R₃, and R₄.

61. The composition of any one of claims 1-60, further comprising a pharmaceutically acceptable carrier.

62. The composition of any one of claims 1-60, further comprising a delivery vehicle.

63. The composition of claim 62, wherein the delivery vehicle is selected from a liposome, a nanoparticle, and a polysaccharide.

64. The composition of claim 63, wherein the polysaccharide is selected from cyclodextrin, chitosan, cellulose, and alginate.

65. The composition of any one of claims 1-64, wherein the composition is formulated for intranasal administration.

66. The composition of claim 65, wherein the composition comprises at least one inhibitor of nasal mucosa proteases.

67. The composition of claim 66, wherein the inhibitor is selected from bestatine, comostate amylase, leupeptin, aprotinin, bacitracin, amastatine, boroleucine, puromycin, a bile salt, and a fusidic acid.

68. The composition of any one of claims 1-64, wherein the composition is formulated for administration by inhalation.

69. The composition of claim 68, wherein the administration by inhalation is performed using a dry powder intranasal device.

70. The composition of any one of claims 1-64, wherein the composition is formulated for intravenous administration.

71. The composition of any one of claims 1-64, wherein the composition is formulated for oral administration.

72. The composition of any one of claims 1-71, wherein the peptide modulates the mGluR₅ receptor (GRM5).

73. A pharmaceutical composition comprising a therapeutically effective amount of the composition of any one of claims 1-72 and at least one pharmaceutically acceptable carrier, diluent, or excipient.

74. A method for modulating mGluR₅ (GRM5) receptor in a cell, comprising contacting the cell with the composition of any one of claims 1-72.

75. A method for treating a mood disorder in a patient in need thereof, comprising administering a therapeutically effective amount of the composition of any one of claims 1-72 to a patient in need thereof.

76. The method of claim 75, wherein the mood disorder is depression.

77. The method of claim 76, wherein the depression is selected from major depressive disorder, dysthymia, breakthrough depression, treatment-refractory depression, and depression associated with Parkinson's disease, depression associated with post-traumatic stress disorder, post-partum depression, bipolar depression.

78. The method of claim 75, wherein the mood disorder is an anxiety disorder.

79. The method of claim 78, wherein the anxiety disorder is selected from generalized anxiety disorder, social anxiety disorder, and panic disorder, post-traumatic stress disorder.

80. The method of claim 75, wherein the mood disorder is schizophrenia.

81. The method of claim 75, wherein the mood disorder is a panic disorder.

82. The method of claim 75, wherein the mood disorder is stress-related disorder.

83. The method of claim 75, wherein the mood disorder is a bipolar disorder.

84. A method for treating a movement disorder in a patient in need thereof, comprising administering a therapeutically effective amount of the composition of any one of claims 1-72 to a patient in need thereof.

85. The method of claim 84, wherein the movement disorder is a hypokinetic movement disorder or a hyperkinetic movement disorder.

86. The method of claim 84, wherein the movement disorder is a movement disorder accompanying a mental disorder.

87. The method of claim 85, wherein the hypokinetic movement disorder is selected from Parkinson's disease, Hallevorden-Spatz disease, progressive supranuclear ophthalmoplegia, and striatonigral deneneration.

88. The method of claim 85, wherein the hyperkinetic movement disorder is selected from dystonia, drug induced dystonia, idiopathic familial dystonia, idiopathic nonfamilial dystonia, spasmodic torticollis, ideopathic orofacial dystonia, blepharospasm, essential tremor, drug induced tremor, myoclonus, opsoclonus, chorea, drug induced chorea, rheumatic chorea (Sydenham's chorea), Huntington's chorea, ballismus, hemiballismus, athetosis, dyskinesia, tardive dyskinesia, levodopa-induced dyskinesia, tic disorders, Tourette's syndrome, stereotypic movement disorder, paroxysmal nocturnal limb movement, restless leg syndrome, stiff-person syndrome, and cerebral palsy.

89. The method of claim 87, wherein the Parkinson's disease comprises primary Parkinson's disease, idiopathic Parkinson's disease, secondary Parkinson's disease or Parkinson plus syndrome(s).

90. The method of claim 84, wherein the movement disorder comprises dystonia, catatonia, essential tremor, Huntington's chorea, Tourette's syndrome, and stereotypic movement disorder.

91. The method of claim 84, the movement disorder can be attention deficit hyperactivity disorder.

92. A method for treating a neurodegenerative disorder in a patient in need thereof, comprising administering a therapeutically effective amount of the composition of any one of claims 1-72 to a patient in need thereof.

93. The method of claim 92, wherein the neurodegenerative disorder is Parkinson's disease.

94. The method of claim 93, wherein the Parkinson's disease comprises primary Parkinson's disease, idiopathic and secondary Parkinson's disease, or Parkinson-plus syndromes.

95. The method of claim 92, wherein the neurodegenerative disorder is Alzheimer's disease.

96. The method of claim 92, wherein the neurodegenerative disorder is associated with a movement disorder.

97. The method of any one of claims 74-96, wherein the method further comprises administering an antidepressant, wherein the antidepressant is optionally selected from the group consisting of serotonin reuptake inhibitors, selective norepinephrine reuptake inhibitors, combined action SSRI/SNRI, serotonin-2 antagonist/reuptake inhibitors, an antidepressant with alpha-2 antagonism plus serotonin-2 and serotonin-3 antagonism, an antidepressant with serotonin/norepinephrine/dopamine reuptake inhibition, an antidepressant with norepinephrine and dopamine reuptake inhibition, 5-HT-1alpha antagonist, 5-HT-1beta antagonist, 5-HT1A receptor agonists, 5-HT1A receptor agonists and antagonists, 5-HT2 receptor antagonists, viloxazine hydrochloride, dehydroepiandosterone, NMDA receptor antagonists, AMPA receptor potentiators, substance P antagonists/neurokinin-1 receptor antagonists, nonpeptide Substance P antagonist, neurokinin 2 antagonists, neurokinin 3 antagonists, corticotropin-releasing factor receptor antagonists, antiglucocorticoid medications, glucocorticoid receptor antagonists, cortisol blocking agents, nitric oxide synthesize inhibitors, inhibitors of phosphodiesterase, enkephalinase inhibitors, GABA-A receptor agonists, free radical trapping agents, atypical MAOI's, selective MAOI inhibitors, hormones, folinic acid, leucovorin, tramadol, and tryptophan in combination with an antipsychotic drug, wherein said antipsychotic drug is selected from the group consisting of an atypical antipsychotic drug, and a dopamine system stabilizer.

98. The method of any one of claims 74-97, wherein the method further comprises administering an additional depression treatment comprising an agent optionally selected from one or more of CYMBALTA oral, LEXAPRO oral, EFFEXOR XR oral, ZOLOFT oral, CELEXA oral, TRAZODONE oral, PROZAC oral, WELLBUTRIN XL oral, CITALOPRAM oral, PRISTIQ oral, AMITRIPTYLINE oral, SAVELLA oral, VIIBRYD oral, PAXIL CR oral, WELLBUTRIN oral, PAXIL oral, SERTRALINE oral, REMERON oral, NORTRIPTYLINE oral, VENLAFAXINE oral, FLUOXETINE oral, BUPROPION HCL oral, MIRTAZAPINE oral, RITALIN oral, PAROXETINE oral, WELLBUTRIN SR oral, DOXEPIN oral, METHYLPHENIDATE oral, SYMBYAX oral, ESCITALOPRAM OXALATE oral, PAMELOR oral, IMIPRAMINE oral, BRINTELLIX oral, DULOXETINE oral, NARDIL oral, FETZIMA oral, EMSAM TRANSDERMAL, PARNATE oral, PEXEVA oral, BRISDELLE oral, CLOMIPRAMINE oral, ANAFRANIL oral, TOFRANIL oral, FLUVOXAMINE oral, ZYBAN oral, DESIPRAMINE oral, SARAFEM oral, PROZAC WEEKLY oral, APLENZIN oral, METHYLIN oral, NEFAZODONE oral, QUILLIVANT XR oral, TOFRANIL-PM oral, NORPRAMIN oral, REMERON SOLTAB oral, BUPROPION HBR oral, OLEPTRO ER oral, DESVENLAFAXINE SUCCINATE oral, BUPROBAN oral, IMIPRAMINE PAMOATE oral, VILAZODONE oral, MILNACIPRAN oral, PAROXETINE MESYLATE oral, SURMONTIL oral, MAPROTILINE oral, PROTRIPTYLINE oral, PHENELZINE oral, MARPLAN oral, OLANZAPINE-FLUOXETINE oral, TRANYLCYPROMINE oral, SELEGILINE TRANSDERMAL, AMOXAPINE oral, FORFIVO XL oral, ISOCARBOXAZID oral, DESVENLAFAXINE oral, KHEDEZLA oral, LEVOMILNACIPRAN oral, VORTIOXETINE oral, and DESVENLAFAXINE FUMARATE oral.

99. The method of any one of claims 84-98, wherein the method further comprises administering an additional agent selected from one or more of LEVODOPA, CARBIDOPA, SAFINAMIDE, PRAMIPEXOLE, ROTIGOTINE, ROPINIROLE, AMANTADINEM, BENZTROPINE, TRIHEXYPHENIDYL, SELEGILINE, RASAGILINE, ENTACAPONE, TOLCAPONE, DIAZEPAM, CLONAZEPAM, BACLOFEN, TRIHEXYPHENIDYL, BENZTROPINE, ETHOPROPAZINE, LORAZEPAM, BROMOCRIPTINE, TETRABENAZINE, PROPRANOLOL, PRIMIDONE, FLUPHENAZINE, HALOPERIDOL, RISPERIDONE, PIMOZIDE, ZIPRASIDONE, FLUPHENAZINE, AMPHETAMINE, METHYLPHENIDATE, DEXMETHYLPHENIDATE, METHYLPHENIDATE, ATOMOXETINE HYDROCHLORIDE, and LISDEXAMFETAMINE DIMESYLATE.

100. The method of any one of claims 74-99, wherein the method further comprises administering an additional anxiety treatment optionally selected from agent one or more of benzodiazepines selected from alprazolam (XANAX), clonazepam (KLONOPIN), diazepam (VALIUM), lorazepam (ATIVAN), oxazepam (SERAX), and chlordiazepoxide (librium); beta blockers selected from propranolol (INDERAL) and atenolol (TENORMIN); tricyclic antidepressants selected from imipramine (TOFRANIL), desipramine (NORPRAMIN, PERTOFRANE), nortriptyline (AVENTYL or PAMELOR), amitriptyline (ELAVIL), doxepin (SINEQUAN or ADAPIN), clomipramine (ANAFRANIL); monoamine oxidase inhibitors (MAOIs) selected from phenelzine (NARDIL), tranylcypromine (PARNATE); selective serotonin reuptake inhibitors (SSRIs) selected from fluoxetine (PROZAC), fluvoxamine (LUVOX), sertraline (ZOLOFT), paroxetine (PAXIL), escitalopram oxalate (LEXAPRO), citalopram (CELEXA); serotonin-norepinephrine reuptake inhibitors (SNRIs) selected from venlafaxine (EFFEXOR), venlafaxine extended release (EFFEXOR XR) and duloxetine (CYMBALTA); mild tranquilizers such as buspirone (BUSPAR); and anticonvulsants selected from valproate (DEPAKOTE), pregabalin (LYRICA), and gabapentin (NEURONTIN).
